(19) United States
(12) Patent Application Publication
(10) Pub. No.: US 2025/0340890 A1
(43) Pub. Date: Nov. 6, 2025

(54) DNA APTAMERS AS UNIVERSAL INHIBITORS OF SPIKE PROTEIN/HACE2 INTERACTIONS

(71) Applicant: Bowling Green State University, Bowling Green, OH (US)

(72) Inventors: Xiaohong Tan, Bowling Green, OH (US); Achut Prasad Silwal, Bowling Green, OH (US)

(73) Assignee: Bowling Green State University, Bowling Green, OH (US)

(21) Appl. No.: 18/869,471

(22) PCT Filed: May 26, 2023
(86) PCT No.: PCT/US2023/067521

§ 371 Date: Nov. 26, 2024

Related U.S. Application Data
(60) Provisional application No. 63/346,534, filed on May 27, 2022.

(51) Int. Cl. C12N 15/115; A61P 31/14

(52) U.S. Cl. CPC C12N 15/115; A61P 31/14; C12N 2310/16; C12N 2320/30

(57) ABSTRACT

Compositions, methods of inhibiting binding between a coronavirus and a MACE2 receptor, methods of treating a coronavirus infection, methods of diagnosing a coronavirus infection, and kits for diagnosing a coronavirus infection, all involving contacting a hACE2 receptor with a DNA aptamer to block binding to the receptor, wherein the DNA aptamer binds to a spike protein of the coronavirus, are described.

### RELATED APPLICATIONS

This application claims priority to U.S. Provisional Application No. 63/346,534 filed under 35 U.S.C. § 111(b) on May 27, 2022, the disclosure of which is incorporated herein by reference in its entirety.

### STATEMENT REGARDING FEDERALLY SPONSORED RESEARCH

This invention was made with government support under Grant Number 2028531 awarded by the National Science Foundation. The government has certain rights in this invention.

### SEQUENCE LISTING

The instant application contains a Sequence Listing which has been submitted electronically in XML format and is hereby incorporated by reference in its entirety. Said XML copy, created on May 25, 2023, is named 63572-WO-PCT_IDN216_SL.xml and is 29,933 bytes in size.

### BACKGROUND

The Coronavirus Disease 2019 (COVID-19) pandemic, caused by severe acute respiratory syndrome coronavirus 2 (SARS-CoV-2), has presented one of the most dangerous global health care challenges in modern history. SARS-CoV-2 uses its homotrimer spike protein (S protein) to attach to the host cell via human angiotensin converting enzyme 2 (hACE2). In humans, this attachment subsequently results in leukocytic infiltration, increased blood vessel permeability, alveolar wall permeability, and decreased secretion of lung surfactants. These adverse effects cause many respiratory problems. Moreover. the exacerbation of local inflammation causes a cytokine storm, eventually leading to a systemic inflammatory response syndrome.

Nobody knows when and where the next coronavirus outbreak will be. Therefore, it is necessary to develop SARS-CoV-2 inhibitors for variants or even a new coronavirus.

### SUMMARY

Provided herein is a method of inhibiting binding between a coronavirus and a hACE2 receptor, the method comprising contacting a hACE2 receptor with a DNA aptamer to block binding to the hACE2 receptor, wherein the DNA aptamer binds to a spike protein of the coronavirus.

In certain embodiments, the spike protein has a S1 subunit, a S2 subunit, and a S1S2 junction, and the DNA aptamer is specific for the S1 subunit, the S2 subunit, or the S1S2 junction.

In certain embodiments, the DNA aptamer comprises S2A2C1, having a nucleotide sequence of AGGCGGGTTCCTAGACTTGTACTCAGCCT (SEQ ID NO: 1).

In certain embodiments, the DNA aptamer comprises A1C1, having a nucleotide sequence of CGGGACGACGACGGACATCGTGAGAAATGGTCGACCTTGTGTCTGTCGTCCCG (SEQ ID NO: 2).

In certain embodiments, the DNA aptamer comprises a fusion aptamer. In particular embodiments, the fusion aptamer comprises a S1-specific aptamer fused to a S2-specific aptamer by a linker. In particular embodiments, the fusion aptamer comprises S1B6C3, having a nucleotide sequence of CGCAGCACCCAAGAACAAGGACTGCTTAGGATTGCGATAGGTTCGG (SEQ ID NO: 3), linked to an additional aptamer. In particular embodiments, the fusion aptamer comprises S2A2C1, having a nucleotide sequence of AGGCGGGTTCCTAGACTTGTACTCAGCCT (SEQ ID NO: 1), linked to an additional aptamer. In particular embodiments, the fusion aptamer comprises S1B6C3, having a nucleotide sequence of CGCAGCACCCAAGAACAAGGACTGCTTAGGATTGCGATAGGTTCGG (SEQ ID NO: 3), linked to S2A2C1, having a nucleotide sequence of AGGCGGGTTCCTAGACTTGTACTCAGCCT (SEQ ID NO: 1). In particular embodiments, the linker comprises a poly A linker.

In certain embodiments, the DNA aptamer comprises S1B6C3-A5-S2A2C1, wherein S1B6C3 has a nucleotide sequence of CGCAGCACCCAAGAACAAGGACTGCTTAGGATTGCGATAGGTTCGG (SEQ ID NO: 3), S2A2C1 has a nucleotide sequence of AGGCGGGTTCCTAGACTTGTACTCAGCCT (SEQ ID NO: 1), and A5 is a poly A linker.

In certain embodiments, the hACE2 receptor is in a human subject.

In certain embodiments, the hACE2 receptor is contacted with a plurality of the DNA aptamers.

In certain embodiments, the coronavirus is SARS-CoV-2. In certain embodiments, the coronavirus is the Delta variant of SARS-CoV-2. In certain embodiments, the coronavirus is the Omicron variant of SARS-CoV-2.

Further provided is a method of treating a coronavirus infection, the method comprising administering to a subject having a coronavirus infection an effective amount of a DNA aptamer to inhibit binding between the coronavirus and hACE2 receptors in the subject so as to treat the coronavirus infection.

In certain embodiments, the subject is a human subject.

In certain embodiments, the coronavirus infection is caused by SARS-CoV-2. In certain embodiments, the coronavirus infection is caused by the Delta variant of SARS-CoV-2. In certain embodiments, the coronavirus infection is caused by the Omicron variant of SARS-CoV-2.

In certain embodiments, the DNA aptamer comprises S2A2C1, having a nucleotide sequence of AGGCGGGTTCCTAGACTTGTACTCAGCCT (SEQ ID NO: 1).

In certain embodiments, the DNA aptamer comprises A1C1, having a nucleotide sequence of CGGGACGACGACGGACATCGTGAGAAATGGTCGACCTTGTGTCTGTCGTCCCG (SEQ ID NO: 2).

In certain embodiments, the DNA aptamer comprises a fusion aptamer. In particular embodiments, the fusion aptamer comprises a S1-specific aptamer fused to a S2-specific aptamer by a linker. In particular embodiments, the fusion aptamer comprises S1B6C3, having a nucleotide sequence of CGCAGCACCCAAGAACAAGGACTGCTTAGGATTGCGATAGGTTCGG (SEQ ID NO: 3), linked to an additional aptamer. In particular embodiments, the fusion aptamer comprises S2A2C1, having a nucleotide sequence of AGGCGGGTTCCTAGACTTGTACTCAGCCT (SEQ ID NO: 1), linked to an additional aptamer. In particular embodiments, the fusion aptamer comprises S1B6C3, having a nucleotide sequence of CGCAGCACCCAAGAACAAGGACTGCTTAGGATTGCGATAGGTTCGG (SEQ ID NO: 3), linked to S2A2C1, having a nucleotide sequence of AGGCGGGTTCCTAGACTTGTACTCAGCCT (SEQ ID NO: 1). In particular embodiments, the linker comprises a poly A linker.

In certain embodiments, the DNA aptamer comprises S1B6C3-A5-S2A2C1, wherein S1B6C3 has a nucleotide sequence of CGCAGCACCCAAGAACAAGGACTGCTTAGGATTGCGATAGGTTCGG (SEQ ID NO: 3), S2A2C1 has a nucleotide sequence of AGGCGGGTTCCTAGACTTGTACTCAGCCT (SEQ ID NO: 1), and A5 is a poly A linker.

Further provided is a method of diagnosing a coronavirus infection, the method comprising obtaining a sample from a subject; contacting the sample with a DNA aptamer specific for a spike protein of a coronavirus; and analyzing an extent of binding between the DNA aptamer and the sample to determine if the coronavirus is present in the sample, wherein binding between the DNA aptamer and the sample indicates a coronavirus is present in the sample, so as to diagnose whether the subject has a coronavirus infection.

In certain embodiments, the sample comprises mucus from a nose of the subject.

In certain embodiments, the DNA aptamer comprises S2A2C1, having a nucleotide sequence of AGGCGGGTTCCTAGACTTGTACTCAGCCT (SEQ ID NO: 1).

In certain embodiments, the DNA aptamer comprises A1C1, having a nucleotide sequence of CGGGACGACGACGGACATCGTGAGAAATGGTCGACCTTGTGTCTGTCGTCCCG (SEQ ID NO: 2).

In certain embodiments, the DNA aptamer comprises a fusion aptamer. In particular embodiments, the fusion aptamer comprises a S1-specific aptamer fused to a S2-specific aptamer by a linker. In particular embodiments, the fusion aptamer comprises S1B6C3, having a nucleotide sequence of CGCAGCACCCAAGAACAAGGACTGCTTAGGATTGCGATAGGTTCGG (SEQ ID NO: 3), linked to an additional aptamer. In particular embodiments, the fusion aptamer comprises S2A2C1, having a nucleotide sequence of AGGCGGGTTCCTAGACTTGTACTCAGCCT (SEQ ID NO: 1), linked to an additional aptamer. In particular embodiments, the fusion aptamer comprises S1B6C3, having a nucleotide sequence of CGCAGCACCCAAGAACAAGGACTGCTTAGGATTGCGATAGGTTCGG (SEQ ID NO: 3), linked to S2A2C1, having a nucleotide sequence of AGGCGGGTTCCTAGACTTGTACTCAGCCT (SEQ ID NO: 1). In particular embodiments, the linker comprises a poly A linker.

In certain embodiments, the DNA aptamer comprises S1B6C3-A5-S2A2C1, wherein S1B6C3 has a nucleotide sequence of CGCAGCACCCAAGAACAAGGACTGCTTAGGATTGCGATAGGTTCGG (SEQ ID NO: 3), S2A2C1 has a nucleotide sequence of AGGCGGGTTCCTAGACTTGTACTCAGCCT (SEQ ID NO: 1), and A5 is a poly A linker.

Further provided is a composition comprising a fusion aptamer comprising S1B6C3-A5-S2A2C1, wherein S1B6C3 has a nucleotide sequence of CGCAGCACCCAAGAACAAGGACTGCTTAGGATTGCGATAGGTTCGG (SEQ ID NO: 3), S2A2C1 has a nucleotide sequence of AGGCGGGTTCCTAGACTTGTACTCAGCCT (SEQ ID NO: 1), and A5 is a poly A linker. In certain embodiments, the composition further comprises a pharmaceutically acceptable carrier, diluent, or adjuvant.

Further provided is a composition comprising at least two of (i) an aptamer comprising S2A2C1, having a nucleotide sequence of AGGCGGGTTCCTAGACTTGTACTCAGCCT (SEQ ID NO: 1), (ii) an aptamer comprising A1C1, having a nucleotide sequence of CGGGACGACGACGGACATCGTGAGAAATGGTCGACCTTGTGTCTGTCGTCCCG (SEQ ID NO: 2), and (iii) an aptamer comprising S1B6C3-A5-S2A2C1, wherein S1B6C3 has a nucleotide sequence of CGCAGCACCCAAGAACAAGGACTGCTTAGGATTGCGATAGGTTCGG (SEQ ID NO: 3), S2A2C1 has a nucleotide sequence of AGGCGGGTTCCTAGACTTGTACTCAGCCT (SEQ ID NO: 1), and A5 is a poly A linker. In certain embodiments, the composition further comprises a pharmaceutically acceptable carrier, diluent, or adjuvant.

Further provided is a kit for diagnosing a coronavirus infection, the assay comprising a first container housing a solution comprising a DNA aptamer specific for a spike protein of a coronavirus; and a second container housing an instrument for collecting a sample from a subject.

In certain embodiments, the DNA aptamer comprises S2A2C1, having a nucleotide sequence of AGGCGGGTTCCTAGACTTGTACTCAGCCT (SEQ ID NO: 1).

In certain embodiments, the DNA aptamer comprises A1C1, having a nucleotide sequence of CGGGACGACGACGGACATCGTGAGAAATGGTCGACCTTGTGTCTGTCGTCCCG (SEQ ID NO: 2).

In certain embodiments, the DNA aptamer comprises a fusion aptamer. In particular embodiments, the fusion aptamer comprises a S1-specific aptamer fused to a S2-specific aptamer by a linker. In particular embodiments, the fusion aptamer comprises S1B6C3, having a nucleotide sequence of CGCAGCACCCAAGAACAAGGACTGCTTAGGATTGCGATAGGTTCGG (SEQ ID NO: 3), linked to an additional aptamer. In particular embodiments, the fusion aptamer comprises S2A2C1, having a nucleotide sequence of AGGCGGGTTCCTAGACTTGTACTCAGCCT (SEQ ID NO: 1), linked to an additional aptamer. In particular embodiments, the fusion aptamer comprises S1B6C3, having a nucleotide sequence of CGCAGCACCCAAGAACAAGGACTGCTTAGGATTGCGATAGGTTCGG (SEQ ID NO: 3), linked to S2A2C1, having a nucleotide sequence of AGGCGGGTTCCTAGACTTGTACTCAGCCT (SEQ ID NO: 1). In particular embodiments, the linker comprises a poly A linker.

In certain embodiments, the DNA aptamer comprises S1B6C3-A5-S2A2C1, wherein S1B6C3 has a nucleotide sequence of CGCAGCACCCAAGAACAAGGACTGCTTAGGATTGCGATAGGTTCGG (SEQ ID NO: 3), S2A2C1 has a nucleotide sequence of AGGCGGGTTCCTAGACTTGTACTCAGCCT (SEQ ID NO: 1), and A5 is a poly A linker.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B: The interaction of SARS-CoV-2 S protein to hACE2 and fusion aptamers. FIG. 1A shows the interaction of trimeric-S protein and hACE2. The three S1B domains, S1A domains, and S2 subunits of the trimeric S-protein are depicted in yellow, cyan, and silver colors, respectively. The hACE2 (PBD code 6mOj) is depicted in purple color. FIG. 1B shows a demonstration of SARS-CoV-2 neutralization by using the fusion aptamers described herein. In a single fusion aptamer, the S1B aptamer. linker, and the S2 aptamer are depicted in red, black, and purple colors, respectively.

FIGS. 2A-2H: Selection and characterization of anti-S2 DNA aptamers. FIG. 2A shows the SELEX scheme for the selection of aptamers against the S2 domain of the WT S-protein. FIG. 2B shows a schematic representation of 6-FAM-labeled DNA binding to the target-protein/bead complex (not to scale). FIG. 2C shows enrichment of the binding capability of the ssDNA library over various selection rounds. The fluorescence emission was measured with a Clariostar microplate reader at λₘₐₓ=520 nm. FIG. 2D shows a schematic representation of 6-FAM-labeled DNA bound to the target protein for fluorescence microscopy. FIG. 2E shows fluorescence imaging of S2-protein/Ni-NTA resin beads in the presence of the 6-FAM-labeled library obtained from the 10^{th}, 11^{th}, and 15^{th} rounds (encircled red dots). This shows that the S2-specific pools are satisfactorily enriched after the 15^{th} round of selection. The control protein/Ni-NTA resin bead does not produce a fluorescence signal in the presence of the 6-FAM-labeled library obtained from the 15^{th} round of selection. Both the transmitted light (top panel) and green fluorescence channel (bottom panel) images were collected using a digital inverted fluorescence microscope (Invitrogen EVOS FL). FIG. 2F shows binding affinities (K_{d}) of the original and truncated anti-S2 aptamers. FIG. 2G shows secondary structures of S2 aptamers obtained using NUPACK. The aptamers S2A1 and S2A2 are the original anti-S2 candidates, while S2A1C1, S2A2C1, and S2A2C3 are the optimized truncated aptamers obtained from the S2A1 and S2A2 aptamers by the deletion of redundant nucleotides, respectively. FIG. 2G discloses SEQ ID NOS 4, 7, 5, 1 and 8, respectively, in order of appearance. FIG. 2H shows the nucleotide sequences of all the anti-S2 aptamers. The nucleotides removed in the optimized aptamers are represented by dotted lines. FIG. 2H discloses SEQ ID NOS 4, 7, 5, 1, and 8, respectively, in order of appearance.

FIGS. 3A-3G: FIG. 3A shows a schematic representation of 6-FAM-labeled DNA binding to the target-protein/bead complex (not to scale). FIG. 3B shows a schematic representation of 6-FAM-labeled DNA bound to the target protein for fluorescence microscopy. FIG. 3C shows a scheme showing that S2A2C1 avoids aggregation and preserves the red wine color of the AuNPs colloids (left panel). When the specific targets (S2- or spike-protein) are added, S2A2C1 preferentially binds to the specific target, leaving AuNPs for aggregation and producing the purple color (right panel). FIG. 3D shows the ratio of the absorbances (A520 nm/620 nm) quantifies specific binding. When A520 nm/620 nm is smaller than 1, it indicates specific binding, while a larger ratio indicates the absence or weaker binding. FIG. 3E shows a schematic representation of the ELISA-based inhibition efficacy measurement (not to scale). The hACE2 was coated onto the plate. When no aptamer or control aptamer is present with the spike-protein, a color product was formed; the chromogenic reagent (HRP+TMB) is encircled by the dotted line (upper panel). When an aptamer inhibits the His-tagged spike-protein/hACE2 interaction, the anti-His tagged HRP no longer persists after washing and does not impart color to the TMB (lower panel). FIGS. 3F-3G show the specificity test for the anti-S2 aptamer S2A2C1 using an AuNPs-based colorimetric assay. FIG. 3F shows the addition of non-specific targets such as 250 nM solution of PBS, BSA, S1-protein, lysozyme, and PD-L1 to colloids of AuNPs, 1.5 M NaCl, and 250 nM S2A2C1 aptamer does not affect the S2A2C1 aptamer, hence the red wine color persists for more than 48 h. When 250 nM solution of S2- or spike-protein was added, it removed the aptamer from the AuNPs colloids, and the purple color was observed within 5 minutes of addition. FIG. 3G shows UV-Vis absorption spectra of AuNP solutions containing the 250 nM of S2A2C1 aptamer after addition of 250 nM of spike-protein, S2-protein, S1-protein, BSA, PBS-buffer, Lysozyme, and PD-L1. The redshift indicates the formation of AuNPs aggregation.

FIGS. 4A-4F: Determination of the binding affinities and inhibition efficacies of aptamers to the WT SARS-CoV-2 S1S2 protein. FIG. 4A shows fluorescence imaging of Ni-NTA resin beads in the presence of the His-tagged WT S1S2 protein and FAM-labeled mono and fusion aptamers. The protein and 6-FAM-labeled aptamers were used in the same concentration of 100 nM. The right-most image shows that Ni-NTA beads do not give a fluorescence signal in the presence of the control protein and S1B6C3-A5-S2A2C1, indicating the aptamer does not bind to the control protein. FIG. 4B shows the binding affinities (K_{d}) of mono and fusion aptamers against the His-tagged WT S1S2 protein. FIG. 4C shows flow cytometry measurements of the green fluorescence emission from magnetic beads in the presence of the WT S1S2 protein and various aptamers. The protein and 6-FAM-labeled aptamers were used in the same concentration of 500 nM. The library with random sequences and the S1B6C3-A5-S2A1C1 fusion aptamers displays the minimum and maximum binding potentials, respectively, to the WT S1S2 protein. FIG. 4D shows a schematic representation of the ELISA-based inhibition efficacy measurement (not to scale). The hACE2 was coated onto the plate. When no aptamer or control aptamer was present with the S1S2-protein, a color product was formed; the chromogenic reagent (HRP+TMB) is encircled by the dotted line (upper panel). When an aptamer inhibits the His-tagged S1S2 protein/hACE2 interaction, the anti-His tagged HRP no longer persists after washing and docs not impart color to the TMB (lower panel). FIG. 4E shows normalized relative absorbances of the final ELISA products corresponding to neutralization efficacies of the various aptamers. The absorbance is inversely related to the neutralization efficacy of the aptamer. FIG. 4F shows the color of the final product in ELISA tests corresponding to various aptamers: F1=S1B6C3-A5-S2A2C1, F2=S2A2C1, F3=S1B6C3-A10-S2A2C1, F4=S1B6C3-A15-S2A2C1, F5=aptamer control, F6=no aptamer, F7=S2A2C1-T15-S1B6C3, F8=S1B6C3, F9=S1B6C3-T15-S2A2C1, F10=S1B6C3-PEG-S2A2C1, F11=TMB only, F12=SELEX buffer. An intense yellow color was obtained due to HRP mediated oxidation of the TMB when an unspecific (control) or no aptamer was added.

FIGS. 5A-5D: FIG. 5A shows the binding affinity of two fusion aptamers, S2A2C1-T15-S1B6C3 and S1B6C3-T15-S2A2C1. The fusion aptamer having S1B6C3 in the 5′ position has better binding affinity than that in the 3′ position. FIG. 5B shows the neutralization efficacy of fusion aptamers having S1B6C3 in the 5′ or 3′ position for polyT linker (T15 (SEQ ID NO: 28) and T25 (SEQ ID NO: 29)) against hACE2/spike-protein interaction. Results show the S1B6C3 in the 5′ position is more compelling. FIG. 5C shows secondary structures of the fusion aptamers by NUPACK (FIG. 5C discloses SEQ ID NOS 9-12, respectively, in order of appearance), and FIG. 5D shows the nucleotide sequences. FIG. 5D discloses SEQ ID NOS 9-12, respectively, in order of appearance.

FIGS. 6A-6B: ELISA data determined the neutralization efficacies of the aptamers against the WT S1S2 protein. FIG. 6A shows photographs of the well plate showing the intensity of the color product formed due to the HRP mediated oxidation of TMB. The top and bottom panels were obtained from the same samples before and after the use of 2 μL of conc. H₂SO₄, respectively. The sample description is as following. A1=Cocktail, A2=S1B6C3-A5-S2A2C1, A3=S2A2C1, A4=S1B6C3-A10-S2A2C1, A5=control aptamer, A6=No aptamer, A7=S2A2C1-T15-S1B6C3, A8=S1B6C3, A9 =S1B6C3-T15-S2A2C1, A10=S1B6C3-PEG-S2A2C1, A11=TMB, A12=SELEX Buffer, A13, A14=S1B6C3-T15-S2A2C1, A15, A16=S1B6C3-T25-S2A2C1, A17, A18=S2A2C1-T25-S1B6C3, A19, A20=S2A2C1-T25-S1S2A1C, A21, A22=No aptamer, A23, A24=TMB. FIG. 6B shows absorbance from the yellow color measured at λₘₐₓ=450 nm. The neutralization efficacies of the aptamers are analyzed as an inversely related property of the absorbance.

FIGS. 7A-7D: Binding affinities and inhibition efficacies of aptamers against variants of SARS-CoV-2 spike-proteins. FIG. 7A shows binding affinity (K_{d}) of the aptamers with the Delta variant spike-protein. FIG. 7B shows inhibition efficacies of aptamers or the control aptamer against Delta spike-protein. Relative normalized absorbances and inhibition efficacies are inversely proportional. FIG. 7C shows inhibition efficacies of the aptamers against the Omicron spike-protein. FIG. 7D shows the comparison of the binding affinities of aptamers towards the WT vs the Delta spike-protein revealing that the aptamers bind to both proteins with comparable affinities.

FIGS. 8A-8B: ELISA data determined the neutralization efficacies of the aptamers against the Delta variant S1S2 protein. FIG. 8A shows photographs of the well plate showing the intensity of the color product formed due to the HRP mediated oxidation of TMB. The top and bottom panels were obtained from the same samples before and after the use of 2 μL of conc. H₂SO₄, respectively. The sample description is as following. A1=S1B6C3-A5-S2A2C1, A2=S1B6C3-A10-S2A2C1, A3=cocktail, A4=S1B6C3-T15-S2A2C1, A5=control aptamer, A6=no aptamer, A8=S1B6C3-A15-S2A2C1, A9=SELEX Buffer, A8=S2A2C1, A9=S1B6C3, A10=TMB, A11=SELEX buffer. FIG. 8B shows absorbance from the yellow color measured at λₘₐₓ=450 nm. The neutralization efficacies of the aptamers are analyzed as an inversely related property of the absorbance.

FIGS. 9A-9B: ELISA data determine the neutralization efficacies of the aptamers against the Omicron variant S-protein. FIG. 9A shows photographs of the well plate showing the intensity of the color product formed due to the HRP mediated oxidation of TMB. The top and bottom panels were obtained from the same samples before and after the use of 2 μL of conc. H₂SO₄, respectively. The sample description is as following. A1=S1B6C3-A5-S2A2C1, A2=S2A2C1, A3=S1B6C3, A4=Random Sequence, A5=no aptamer, A6=cocktail, A7=Control Aptamer, A8=TMB, A9=SELEX Buffer. FIG. 9B shows absorbances from the yellow color measured at λₘₐₓ=450 nm. The neutralization efficacies of the aptamers are analyzed as an inversely related property of the absorbance.

FIG. 10: Gel electrophoresis data assesses the quality of the PCR product to determine the optimized number of PCR cycles (indicated by upward red arrow), required for the DNA amplification. The correct PCR product has 73 base pairs of nucleotides. The DNA ladder or the control (correct PCR product from the first selection round) was used to characterize the size of PCR products. Selection rounds 1-5 have additional 9 PCR cycles, and 6-15 have additional 7 PCR cycles running before the optimization step.

FIGS. 11A-11F: FIG. 11A shows colonies of the ampicillin resistant E. coli bacteria; the purified dsDNA received from the 15^{th} round of selection was ligated with the TOPO vector, and the recombinant DNA was used to transform the E. coli component cell. The single colony of the bacterial was transferred to culture in Luria Broth solution. FIGS. 11B-11F show gel electrophoresis data of the direct PCR analysis using bacterial cells as the templates. The desirable insert has 236 nucleotides. The 1 μL of bacterial culture was used for the PCR, and the remaining was used to extract the purified plasmid for the DNA sequencing. The concentrations of purified plasmids were measured, and 40 plasmid samples with higher concentrations were used for the DNA sequencing.

FIG. 12: Table 3, displaying the sequences of the fusion aptamers used in Example I herein. The nucleotides base of anti-S2, and anti-S1 aptamers are shown in blue and black color, respectively. FIG. 12 discloses SEQ ID NOS 9, 13, 10, 14, 3, 1, 12. 15, and 11, respectively, in order of appearance

FIG. 13: Illustration of a fusion aptamer binding to the spike protein of a coronavirus to prevent interaction between the spike protein and ACE2 receptors on a human cell.

FIGS. 14A-14B: The interaction of SARS-CoV-2 S protein to hACE2 and a DNA aptamer. FIG. 14A shows the interaction of trimeric-S protein and hACE2. The three S1B domains, S1A domains, and S2 subunits of the trimeric S-protein are depicted in cyan, orange, and blue colors, respectively. The hACE2 (PBD code 6mOj) is depicted in magenta color. FIG. 14B shows a demonstration of SARS-CoV-2 neutralization using the A1C1 aptamer (red).

FIGS. 15A-15F: Selection and characterization of anti-S1S2 DNA aptamers. FIG. 15A shows the SELEX scheme for the selection of aptamers against the WT S1S2 protein. FIG. 15B shows the fluorescence intensity of the ssDNA library increases over selection rounds, indicating the enhanced binding capability. The fluorescence emission was measured at λₘₐₓ=520 nm. FIG. 15C shows fluorescence imaging of S1S2-protein/Ni-NTA beads in the presence of the 6-FAM-labeled library. The libraries were obtained from the 0^{th}, 8^{th}, 9^{th}, and 10^{th} rounds, respectively. It shows that the S1S2-specific pool is satisfactorily enriched after the 10^{th} round of selection. The control protein/Ni-NTA does not display a fluorescence signal. FIG. 15D shows the secondary structures of anti-S1S2 aptamers obtained using NUPACK. FIG. 15D discloses SEQ ID NOS 16, 2, and 17 respectively, in order of appearance. FIG. 15E shows the specificity test for the A1C1 aptamer using an AuNPs-based colorimetric assay. The addition of nonspecific targets such as phosphate buffer saline (PBS), bovine serum albumin (BSA), and WT S1 to AuNPs/A1C1/NaCl does not affect the aptamer, hence red-wine color persists. When S1S2 or S2 is added, it removes the A1C1 from the vicinity of AuNPs, and blue or purple color was observed. FIG. 15F shows UV-Vis absorption spectra of AuNP/A1C1/NaCl after addition of WT S1S2, WT S1, WT S2, BSA, or PBS. The redshift from 520 nm indicates the formation of AuNPs aggregation. The aptamers SA1 and SA2 are the original, while A1C1 is optimized from the SA1 by deleting redundant nucleotides (dotted ones). FIG. 15G shows the nucleotide sequences of anti-S1S2 aptamers. FIG. 15G discloses SEQ ID NOS 16, 2, and 17, respectively, in order of appearance.

FIG. 16: The gel electrophoresis data corresponding to the selection process of the A1C1 aptamer. The gel electrophoresis data assesses the quality of the PCR product to determine the optimized number of PCR cycles (indicated by the red arrow) required for the DNA amplification. The correct size of the PCR product had 73 base pairs of nucleotides. The DNA ladder was used to characterize the size of the PCR products. Prior to the optimization, all selection rounds underwent 9 PCR cycles. The PCR cycles/yield relationship over selection rounds (lower right corner) shows that the 10″ selection round has the highest PCR yield.

FIGS. 17A-17B: The schematic representation of the fluorescence emission measurement experiments (not to scale). FIG. 17A shows the 6-FAM-aptamer bound to the His-tagged target protein eluted by a hot SELEX buffer at 95° C. The fluorescence emission intensity from the elution was measured with a Clariostar microplate reader at λₘₐₓ=520 nm. FIG. 17B shows the scheme of ssDNA interaction with the His-tagged protein for fluorescence imaging. The fluorescence images of the 6-FAM-aptamer and Nickel-nitrilotriacetic acid resin bead in the presence of target or control proteins were collected using a digital inverted fluorescence microscope (Invitrogen EVOS FL).

FIGS. 18A-18D: FIGS. 18A-18C show the gel electrophoresis data of the bacterial PCR product; the desirable insert has 236 nucleotides. The 1 μL of bacterial culture was used for the PCR, and the remaining was used to extract the purified plasmids for DNA sequencing. The concentrations of the purified plasmids were measured, and 24 plasmid samples with higher concentrations for the DNA sequencing were used. FIG. 18D shows the ampicillin-resistant bacteria culture in the agar plate. The purified dsDNA received from the 10^{th} round of selection was ligated with the TOPO vector, and the recombinant DNA was used to transform the E. coli component cell. Each single colony of the bacteria was transferred to culture the bacteria in Luria broth solution.

FIG. 19: Schematic representation of gold nanoparticle-based colorimetric assay to measure the specificity of the A1C1 aptamer against WT S1S2. The addition of non-specific targets (PBS, BSA) to AuNPs/NaCl/A1C1 did not affect the AuNPs/aptamer interaction; hence, the red color was intact for more than 48 h (Left). The addition of 250 nM WT S1S2 dramatically changed AuNPs color to purple or blue within 5 minutes, suggesting A1C1 is preferably bound to WT S1S2.

FIGS. 20A-20C: The binding affinity of the A1C1 aptamer against WT (FIG. 20A), Delta (FIG. 20B), or Omicron (FIG. 20C) S1S2 spike protein. In each of FIGS. 20A-20C, the image on the left is a flow cytometry measurement to find the mean green fluorescence emission intensity (X_{C}) from the 6-FAM-labeled A1C1 aptamer bound to Ni-NTA bead/His-tagged S1S2 complex. Each flow cytometry peak is constructed by the integration of 3 trials of experiments and contains 15,000 flow events (5000 events from a trial). The image in the middle is the increment of X_{C} value in decimal number as a function of increased concentration of the 6-FAM-A1C1 aptamer bound to the virtually same quantity of S1S2/Ni-NTA bead complex. The error bars indicate the standard deviation of X_{C} value for individual trials from the mean X_{C} value of three trials (right image in each of FIGS. 20A-20C). The binding affinity (K_{d}) of the 6-FAM labeled A1C1 aptamer against S1S2 is determined by intensity vs concentration plot using the Origin software.

FIGS. 21A-21B: The determination of the inhibition efficacy of the A1C1 aptamer against the WT, Delta, and Omicron SARS-CoV-2 spike protein interaction to hACE2. FIG. 21A shows a schematic representation of the ELISA competition assay (not in scale). The A1C1 aptamer blocks the S1S2/hACE2 interaction (right) which interrupts the color formation. FIG. 21B shows the inhibition efficacy of the A1C1 aptamer as a function of the absorbance measured in the ELISA competition assay. The inhibition efficacy test was performed for three trials for each sample. The error bars indicate the deviation of the absorbance for individual trials from the mean of three trials. The A1C1 aptamer reduced absorbance by approximately 89.1, 87.3, and 85% which was contributed by the WT, Delta, and Omicron S1S2/hACE2 interaction, respectively.

FIG. 22: The ELISA data determined the neutralization efficacy of the aptamer against the Omicron variant S-protein. FIG. 22 shows photographs of the well plate showing the intensity of the color product formed due to HRP mediated oxidation of TMB. The left and right panels were obtained from the same sample before and after the use of 2 μL of concentrated H₂SO₄, respectively. The sample description is as follows: 1=WT S1S2 w/o aptamer, 2=WT S1S2 with the A1C1 aptamer, 3=WT S1S2 with the random sequence, 4=Delta S1S2 w/o aptamer, 5=Delta S1S2 with the A1C1 aptamer, 6=Delta S1S2 with the random sequence, 7=Omicron S1S2 w/o aptamer, 8=Omicron S1S2 with the A1C1 aptamer, 9=Omicron S1S2 with the random sequence, 10=TMB buffer. The absorbance from the yellow color was measured at λₘₐₓ=450 nm. The neutralization efficacy of the aptamer was analyzed as an inversely related property of the absorbance. The absorbance measurement showed that A1C1 aptamer reduces the absorbance by approximately 89.1, 87.3, and 85%, which was contributed by the WT, Delta, and Omicron S1S2/hACE2 interactions, respectively.

FIGS. 23A-23C: Flow cytometry approach to determine the binding affinity of the S1B6C3-A5-S2C2C1 aptamer against WT, Delta, or Omicron spike-protein. FIG. 23A shows flow cytometry measurement finds the mean green fluorescence emission intensity (X_{C}) from the 6-FAM-labeled S1B6C3-A5-S2C2C1 aptamer bound to the complex of Ni-NTA bead and His-tagged spike-protein complex. Each flow cytometry histogram was constructed by the integration of 3 trials of experiments that contain a total of 15,000 flow events (5000 events from a trial). FIG. 23B shows the increment of X_{C} value in decimal number as a function of increased concentration of the 6-FAM labeled S1B6C3-A5-S2C2C1 aptamer bound to virtually the same quantity of spike-protein and Ni-NTA bead complex. The very short error bars indicate the reproducibility of similar X_{C} values for various trials. FIG. 23C shows the binding affinity (K_{d}) of the 6-FAM labeled S1B6C3-A5-S2C2C1 aptamer against the spike-protein was determined by intensity vs concentration plot using the Origin software.

FIG. 24: Table 1, sequences obtained from 40 purified plasmid samples. The randomized sequences of the aptamers are flanked by the reverse (shown in red) and forward primers (shown in green) on their 5′ and 3′ ends, respectively. FIG. 24 discloses SEQ ID NOS 4-5 and 18-23, respectively, in order of appearance.

FIG. 25: Table 6, the sequences obtained from purified plasmid samples. The randomized sequences of the aptamers are flanked by reverse (shown in red) and forward primers (shown in green) on their 5′ and 3′ ends, respectively. FIG. 25 discloses SEQ ID NOS 16-17 and 24-27, respectively, in order of appearance.

### DETAILED DESCRIPTION

Throughout this disclosure, various publications, patents, and published patent specifications are referenced by an identifying citation. The disclosures of these publications, patents, and published patent specifications are hereby incorporated by reference into the present disclosure in their entirety to more fully describe the state of the art to which this invention pertains.

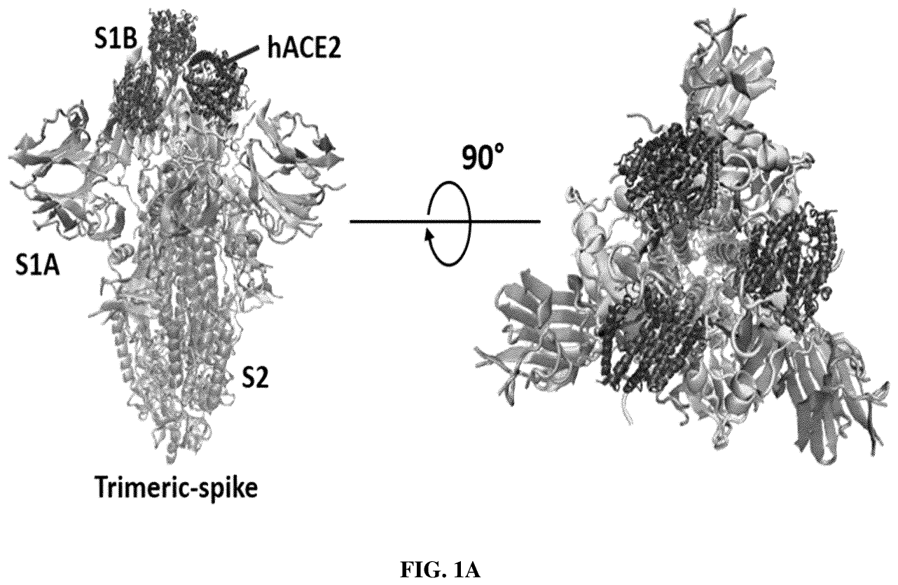

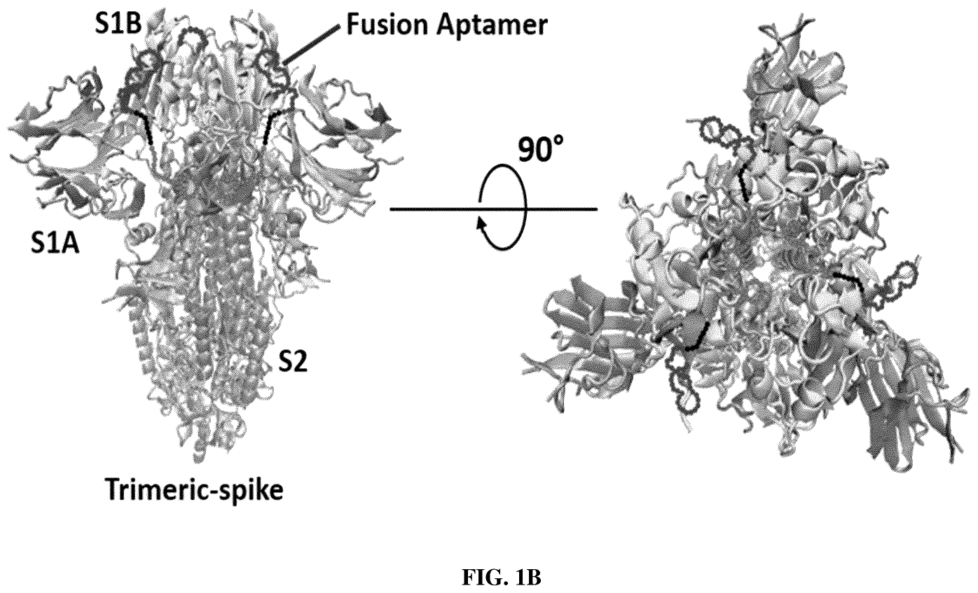

As a strategy to stop the viral infection, the interaction between the S protein of a coronavirus and hACE2 can be blocked. One S protein includes three S1S2 proteins; additionally, each of them has the S1 and S2 subunits, where S1 contains two primary domains S1A and S1B (FIG. 1A). S1A determines the range of the host from the viral particle, and S1B, also known as the receptor-binding domain (RBD), establishes the direct interaction with hACE2 (FIG. 1A). On the other hand, the S2 subunit mediates the fusion of the viral membrane to its potential host cell via the heptad repeat regions. Studies show that virus entry is accomplished via a cascade of events: S1 binds to hACE2, triggering S2 to change from a metastable pre-fusion state to a more stable post-fusion state and allowing viral entry to the host cell. Due to the fact that RBD directly interacts with hACE2, biomolecules such as aptamers can effectively block the RBD/hACE2 interaction.

Aptamers, also called chemical antibodies, are single-stranded oligonucleotides, which can fold into complex 3D structures, enabling them to specifically recognize and bind, through non-covalent interactions, to a large variety of targets such as proteins, nucleic acids, small molecules, or cells. Aptamers are selected from a large pool of random sequences through an iterative selection process called Systematic Evolution of Ligands by Exponential Enrichment (SELEX). Production of DNA aptamers is significantly more cost-effective than making antibodies and can be manufactured using routine chemical synthesis. DNA aptamers are less expensive to produce than antibodies and can be manufactured using general chemical synthesis. DNA aptamers also have lower immunogenicity than antibodies, and low toxicity, making DNA aptamers useful molecular tools in disease therapeutics and diagnostics. The structural stability of the aptamer and aptamer-target complex is usually considered to be responsible for overcoming limitations of aptamer-based therapeutics.

Provided herein are DNA aptamers which are specific for the spike protein of a coronavirus. In some embodiments, the DNA aptamers selectively bind to the spike protein of the SARS-CoV-2 virus, as well as the spike protein of the Delta variant of the SARS-CoV-2 virus, and the spike protein of the Omicron variant of the SARS-CoV-2 virus. In general, the DNA aptamers are aptamers which are specific to the S1, S2, or S1S2 subunits of the spike protein. In some embodiments, an anti-S2 aptamer is conjugated with an anti-S1 aptamer to construct a fusion aptamer that can bind to an S1S2 protein at two different sites. This further enhances binding affinity and inhibition efficacy in blocking the S protein/hACE2 interaction.

As one non-limiting example, the aptamer referred to herein as A1C1, having a nucleotide sequence of CGGGACGACGACGGACATCGTGAGAAATGGTCGACCTTGTGTCTGTCGTCCCG (SEQ ID NO: 2), is an anti-S1S2 aptamer. As shown in the examples herein, the A1C1 aptamer neutralizes the binding of the hACE2 and various S1S2 proteins by 85%-89%. The presence of the A1C1 aptamer reduces absorbance contributed by the S1S2/hACE2 interactions by 89.1% in the WT spike protein, 87.3% in the Delta spike protein, and 85% in the Omicron spike protein. Unlike other aptamers, the A1C1 aptamer binds to the junction domain of S1 and S2. Thus, the A1C1 aptamer is specific to S1S2.

As another non-limiting example, the aptamer referred to herein as S2A2C1, having a nucleotide sequence of AGGCGGGTTCCTAGACTTGTACTCAGCCT (SEQ ID NO: 1), is an anti-S2 aptamer. S2A2C1 is a receptor-binding domain (RBD) independent aptamer which neutralizes the binding of the SARS-CoV-2 spike protein with the hACE2 enzyme on the human cell. As shown in the examples herein, S2A2C1 specifically binds to S2, but not to S1, and has efficacy in blocking the S protein/hACE2 interaction, indicating an RBD independent approach. As shown in the examples herein, in the presence of S2A2C1, only 31% of the Delta S1S2 protein can bind to hACE2.

As another non-limiting example, the aptamer referred to herein as S1B6C3-A5-S2A2C1 is a fusion aptamer composed of the aptamer S1B6C3, having a nucleotide sequence of CGCAGCACCCAAGAACAAGGACTGCTTAGGATTGCGATAGGTTCGG (SEQ ID NO: 3), and the aptamer S2A2C1, having a nucleotide sequence of AGGCGGGTTCCTAGACTTGTACTCAGCCT (SEQ ID NO: 1), fused together by a poly A linker (A5). The S1B6C3 aptamer alone (an anti-S1 aptamer) can effectively neutralize S1S2 and prevent its binding to hACE2, inhibiting 66% of S1S2 binding to hACE2 in the examples herein. However, the fusion aptamer of S1B6C3-A5-S2A2C1 is far superior. As shown in the examples herein, in the presence of the fusion aptamer S1B6C3-A5-S2A2C1, only 8% of the WT spike protein, only 9% of the Delta spike protein, and only 5% of the Omicron (BA.1) spike protein can bind to the human cell receptor enzyme. Because the variants do not cause large conformational changes in the SARS-CoV-2 spike protein, the fusion aptamer S1B6C3-A5-S2A2C1 is a universal inhibitor (i.e., universal for all variants of the SARS-CoV-2 virus). The S1B6C3-A5-S2A2C1 aptamer shows that S2A2C1 can be combined with an existing RBD dependent S1 aptamer, S1B6C3, to increase the inhibition efficacy against SARS-CoV-2.

The S2A2C1, S1B6C3-A5-S2A2C1, and A1C1 aptamers maintain high inhibition efficacy in preventing WT, Delta, and Omicron S1S2 protein binding to hACE2, making them well-suited as diagnostic and therapeutic molecular tools against SARS-CoV-2 and its variants. For instance, the aptamers can be SARS-CoV-2 antibody alternatives for the treatment of a coronavirus infection such as covid-19. The aptamers can also provide point of care diagnostics, being useful in methods and kits for diagnosing a coronavirus infection such as covid-19.

Pharmaceutical compositions of the present disclosure may include an effective amount of a DNA aptamer specific for the spike protein of a coronavirus (i.e., an “active ingredient”), and/or additional agents, dissolved or dispersed in a pharmaceutically acceptable carrier. The phrases “pharmaceutical” or “pharmacologically acceptable” refer to molecular entities and compositions that produce no adverse, allergic, or other untoward reaction when administered to an animal, such as, for example, a human. The preparation of a pharmaceutical composition that contains at least one compound or additional active ingredient will be known to those of skill in the art in light of the present disclosure, as exemplified by Remington's Pharmaceutical Sciences, 2003, incorporated herein by reference. Moreover, for animal (e.g., human) administration, it is understood that preparations should meet sterility, pyrogenicity, general safety, and purity standards as required by FDA Office of Biological Standards.

A composition disclosed herein may comprise different types of carriers depending on whether it is to be administered in solid, liquid or aerosol form, and whether it needs to be sterile for such routes of administration as injection. Compositions disclosed herein can be administered intravenously, intradermally, transdermally, intrathecally, intraarterially, intraperitoneally, intranasally, intravaginally, intrarectally, intraosseously, periprosthetically, topically, intramuscularly, subcutaneously, mucosally, intraosscosly, periprosthetically, in utero, orally, topically, locally, via inhalation (e.g., aerosol inhalation), by injection, by infusion, by continuous infusion, by localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 2003, incorporated herein by reference).

The actual dosage amount of a composition disclosed herein administered to an animal or human patient can be determined by physical and physiological factors such as body weight, severity of condition, the type of disease being treated, previous or concurrent therapeutic interventions, idiopathy of the patient, and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of the active ingredient (i.e., the DNA aptamer specific for the spike protein of a coronavirus) or combination of multiple active ingredients (i.e., multiple different DNA aptamers specific for the spike protein of a coronavirus). In other embodiments, active ingredients may comprise between about 2% to about 75% of the weight of the unit, or between about 25% to about 60%, for example, and any range derivable therein. Naturally, the amount of each active ingredient(s) in each therapeutically useful composition may be prepared in such a way that a suitable dosage will be obtained in any given unit dose of the active ingredient. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In certain embodiments, a composition herein and/or additional agent is formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsules, they may be compressed into tablets, or they may be incorporated directly with the food of the diet.

In further embodiments, a composition described herein may be administered via a parenteral route. As used herein, the term “parenteral” includes routes that bypass the alimentary tract. Specifically. the pharmaceutical compositions disclosed herein may be administered, for example but not limited to, intravenously, intradermally, intramuscularly, intraarterially, intrathecally, subcutaneous, or intraperitoneally (U.S. Pat. Nos. 6,753,514, 6,613,308, 5,466,468, 5,543,158; 5,641,515; and 5,399,363 are each specifically incorporated herein by reference in their entirety).

Solutions of the compositions disclosed herein as free bases or pharmacologically acceptable salts may be prepared in water suitably mixed with a surfactant, such as hydroxypropylcellulose. Dispersions may also be prepared in glycerol, liquid polyethylene glycols and mixtures thereof, and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In some cases, the form should be sterile and should be fluid to the extent that easy injectability exists. It should be stable under the conditions of manufacture and storage and should be preserved against the contaminating action of microorganisms, such as bacteria and fungi. The carrier can be a solvent or dispersion medium containing, for example, water, ethanol, polyol (i.e., glycerol, propylene glycol, liquid polyethylene glycol, and the like), suitable mixtures thereof, and/or vegetable oils. Proper fluidity may be maintained, for example, by the use of a coating, such as lecithin, by the maintenance of the required particle size in the case of dispersion, and/or by the use of surfactants. The prevention of the action of microorganisms can be brought about by various antibacterial and antifungal agents, such as, but not limited to, parabens, chlorobutanol, phenol, sorbic acid, thimerosal, and the like. In some cases, it may be desirable to include isotonic agents, for example, sugars or sodium chloride. Prolonged absorption of the injectable compositions can be brought about by the use in the compositions of agents delaying absorption such as, for example, aluminum monostearate or gelatin.

For parenteral administration in an aqueous solution, for example, the solution may be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed are known to those of skill in the art in light of the present disclosure. For example, one dosage may be dissolved in 1 ml of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

Sterile injectable solutions are prepared by incorporating the compositions in the required amount in the appropriate solvent with various other ingredients enumerated above, as required, followed by filtered sterilization. Generally, dispersions are prepared by incorporating the various sterilized compositions into a sterile vehicle which contains the basic dispersion medium and the required other ingredients from those enumerated above. In the case of sterile powders for the preparation of sterile injectable solutions, some methods of preparation are vacuum-drying and freeze-drying techniques which yield a powder of the active ingredient plus any additional desired ingredient from a previously sterile-filtered solution thereof. A powdered composition is combined with a liquid carrier such as, but not limited to, water or a saline solution, with or without a stabilizing agent.

In other embodiments, the compositions may be formulated for administration via various miscellaneous routes, for example, topical (i.e., transdermal) administration, mucosal administration (intranasal, vaginal, etc.), and/or via inhalation.

Pharmaceutical compositions for topical administration may include the compositions formulated for a medicated application such as an ointment, paste, cream, or powder. Ointments include all oleaginous, adsorption, emulsion, and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones, and laurocapram. Possible bases for compositions for topical application include polyethylene glycol, lanolin, cold cream, and petrolatum, as well as any other suitable absorption, emulsion, or water-soluble ointment base. Topical preparations may also include emulsifiers, gelling agents, and antimicrobial preservatives as necessary to preserve the composition and provide for a homogenous mixture. Transdermal administration of the compositions may also comprise the use of a patch. For example, the patch may supply one or more compositions at a predetermined rate and in a continuous manner over a fixed period of time.

In certain embodiments, the compositions may be delivered by eye drops, intranasal sprays, inhalation, and/or other aerosol delivery vehicles. Methods for delivering compositions directly to the lungs via nasal aerosol sprays has been described in U.S. Pat. Nos. 5,756,353 and 5,804,212 (each specifically incorporated herein by reference in their entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts and could be employed to deliver the compositions described herein. Likewise, transmucosal drug delivery in the form of a polytetrafluoroethylene support matrix is described in U.S. Pat. No. 5,780,045 (specifically incorporated herein by reference in its entirety), and could be employed to deliver the compositions described herein.

It is further envisioned the compositions disclosed herein may be delivered via an aerosol. The term aerosol refers to a colloidal system of finely divided solid or liquid particles dispersed in a liquefied or pressurized gas propellant. The typical aerosol for inhalation is composed of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age and weight, as well as the severity and response of the symptoms.

In particular embodiments, the compositions described herein are useful for treating a coronavirus infection. Accordingly, the compositions may be used in combination therapies. That is, the compositions may be administered concurrently with, prior to, or subsequently to one or more other desired therapeutic or medical procedures or drugs, such as an antibody or other treatment for a coronavirus infection. The particular combination of therapies and procedures in the combination regimen will take into account compatibility of the therapies and/or procedures and the desired therapeutic effect to be achieved. Combination therapies include sequential, simultaneous, and separate administration of the active ingredient in a way that the therapeutic effects of the first administered procedure or drug has not entirely disappeared when the subsequent procedure or drug is administered. By way of a non-limiting example of a combination therapy, the compositions described herein can be administered in combination with one or more treatments for covid-19 such as casirivimab and imdevimab (marketed as REGEN-COV®), or nirmatrelvir and ritonavir (marketed as PAXLOVID™).

In other embodiments, the DNA aptamers described herein are useful for the diagnosis of a coronavirus infection, such as covid-19. The DNA aptamers can be used in a method of diagnosing a coronavirus infection that involves obtaining a sample from a subject, contacting the sample with a DNA aptamer specific for a spike protein of a coronavirus, and analyzing an extent of binding between the DNA aptamer and the sample to determine if the coronavirus is present in the sample, where binding between the DNA aptamer and the sample indicates a coronavirus is present in the sample. Furthermore, the compositions and methods described herein may also be made available via a kit containing one or more key components. A non-limiting example of such a kit comprises a DNA aptamer specific for the spike protein of a coronavirus and an instrument (such as a nasal swab) for obtaining a sample from a subject in separate containers, where the containers may or may not be present in a combined configuration. Many other kits are possible. The kits typically further include instructions for using the components of the kit to practice the subject methods. The instructions for practicing the subject methods are generally recorded on a suitable recording medium. For example, the instructions may be present in the kits as a package insert or in the labeling of the container of the kit or components thereof. In other embodiments, the instructions are present as an electronic storage data file present on a suitable computer readable storage medium, such as a flash drive. In other embodiments, the actual instructions are not present in the kit, but means for obtaining the instructions from a remote source, such as via the internet, are provided. An example of this embodiment is a kit that includes a web address where the instructions can be viewed and/or from which the instructions can be downloaded. As with the instructions, this means for obtaining the instructions is recorded on a suitable substrate.

### EXAMPLES

### Example I—DNA Aptamers Inhibit SARS-Cov-2 Spike Protein Binding to hACE2 by an RBD-Independent or Dependent Approach

Since SARS-CoV-2 uses its surface spike glycoprotein (S protein) to recognize human receptor ACE2 (hACE2), mediating its entry into cells, ligands that can specifically recognize the S protein have the ability to prevent infection. DNA aptamers against the S2 domain of wild-type S protein have been discovered. After refinement, among all candidates, the aptamer S2A2C1 has the shortest sequence and the best binding affinity towards the S2 protein. More importantly, as an aptamer that does not bind to the receptor-binding domain (RBD) of the S protein, S2A2C1 demonstrates efficacy in blocking the S protein/hACE2 interaction, indicating an RBD independent approach. To further improve its binding and inhibition efficacy, S2A2C1 was conjugated with an anti-RBD aptamer, S1B6C3, using suitable linkers to construct fusion aptamers. The refined S1B6C3-A5-S2A2C1 showed the best binding affinity to the S protein, as well as the highest inhibition efficacy against the S protein/hACE2 interaction. Both S2A2C1 and S1B6C3-A5-S2A2C1 maintain high inhibition efficacies to prevent the Delta or Omicron S protein binding to hACE2, rendering them well-suited as diagnostic and therapeutic molecular tools to target SARS-CoV-2 and its variants. This approach, to discover aptamer inhibitors targeting the highly conserved S2 domain, as well as the design of fusion aptamers, can also be used to target any new coronaviruses as they emerge.

DNA SELEX was performed on the wild-type (WT) S2 protein, and several anti-S2 aptamers, including predominant aptamers S2A1 and S2A2, were obtained. Both original aptamers and their truncated offspring aptamers bind to S2 or S1S2 protein with high affinity and specificity. More interestingly, these anti-S2-aptamers can block the S1S2/hACE2 interaction, indicating an RBD independent neutralization mechanism. To further improve the inhibition efficacy, one anti-S2-aptamer (S2A2C1) and one anti-S1B-aptamer (S1B6C3) were used to construct fusion aptamers containing various linkers. The fusion probe, through an RBD-dependent approach, binds to the S1S2 protein at two different sites and shows largely enhanced inhibition efficacy to prevent the S1S2 protein binding to hACE2. This includes the WT, Delta, and Omicron variant S1S2 proteins. In short, this example demonstrates an RBD independent approach (by anti-S2-aptamers) and an RBD dependent approach (by fusion aptamers) to block the S1S2/hACE2 interaction.

### Results and Discussion

### Selection of the S2 Specific DNA Library, and Sequencing Analysis

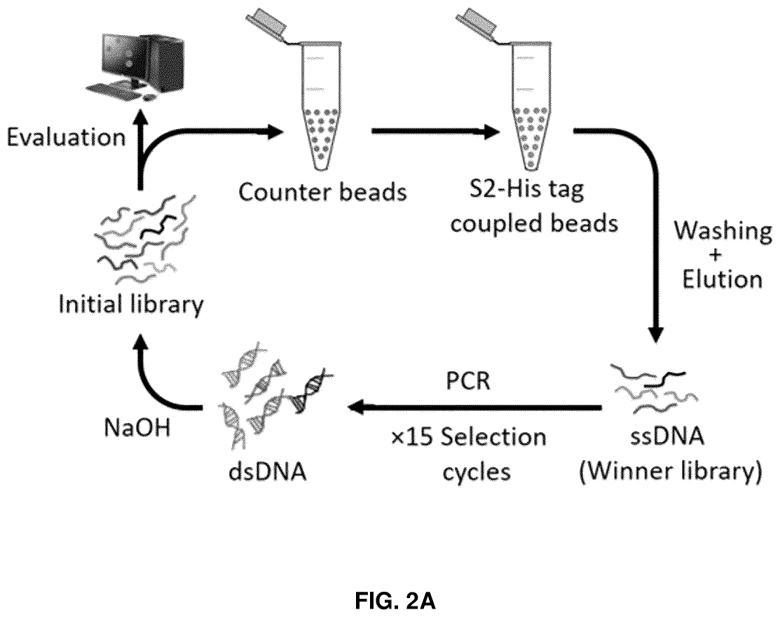

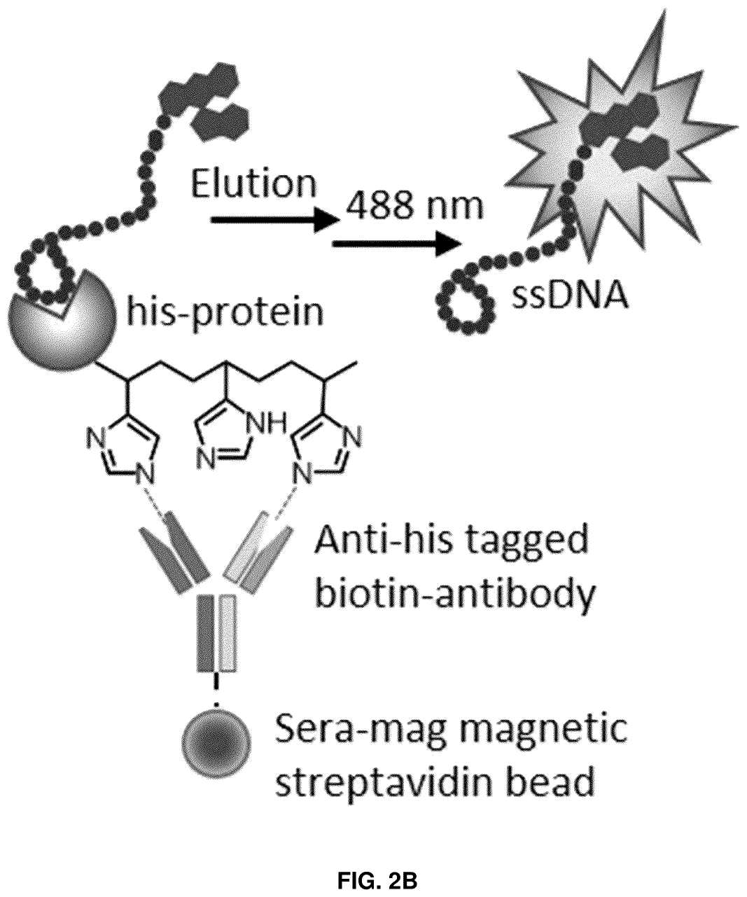

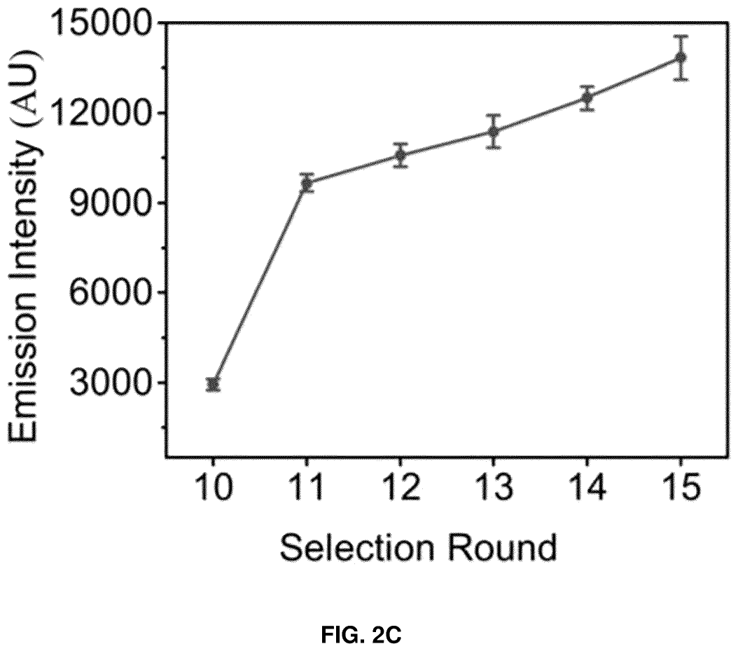

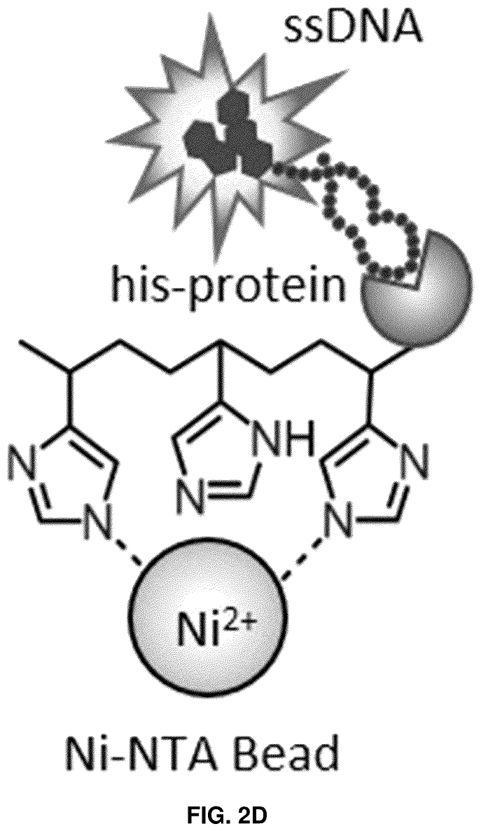

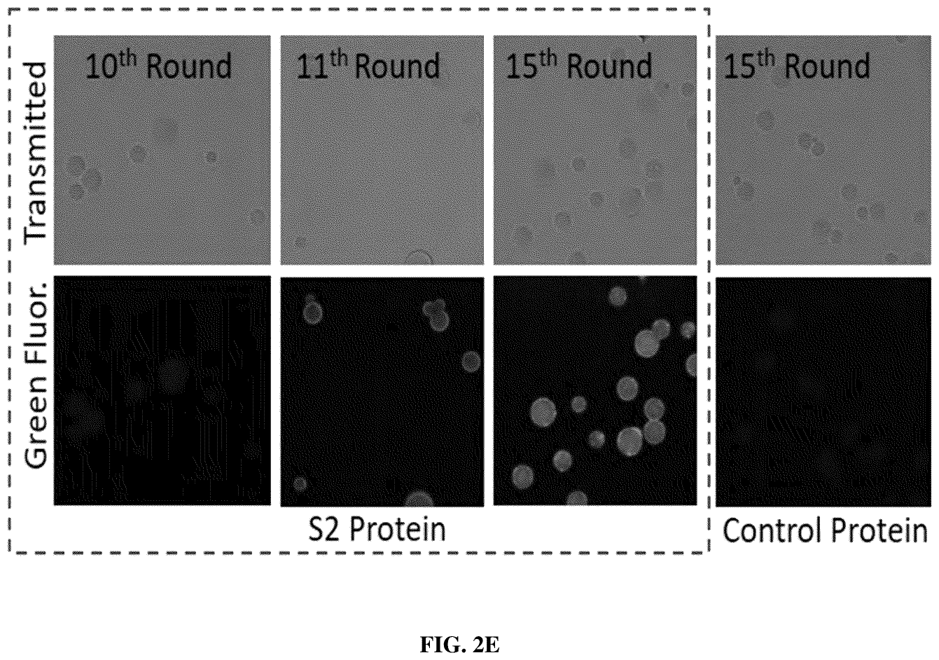

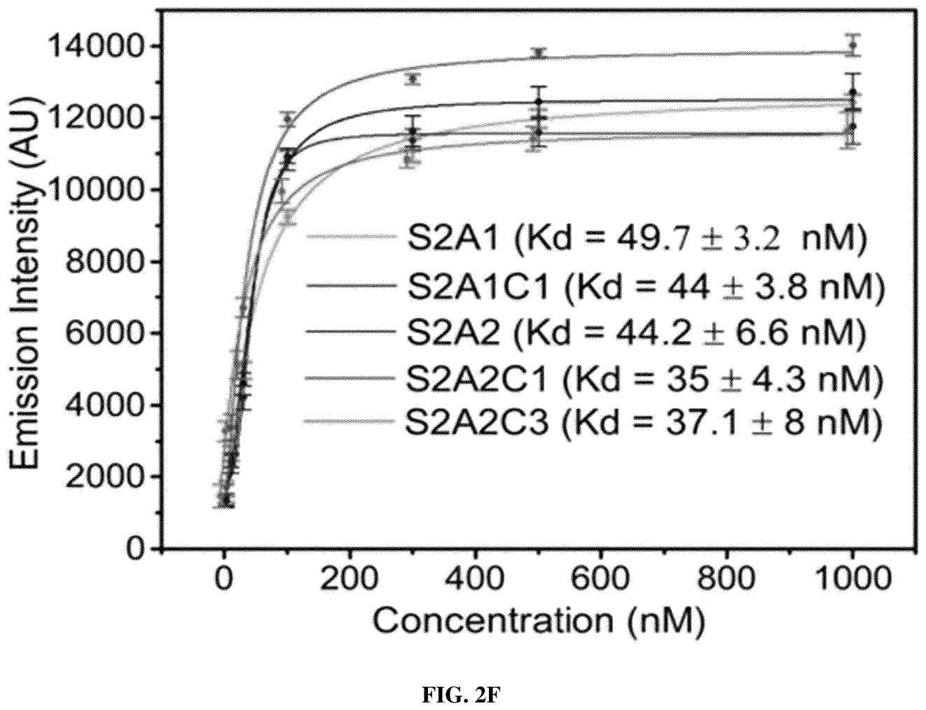

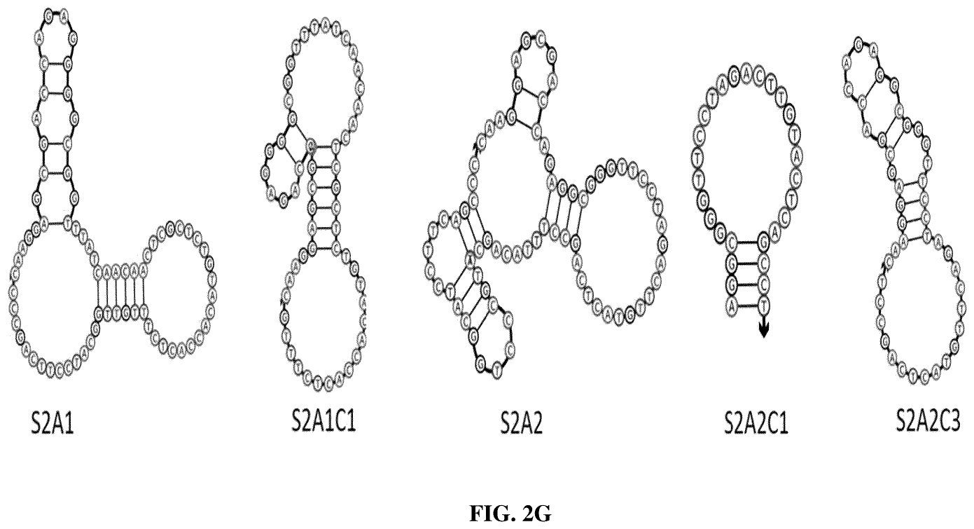

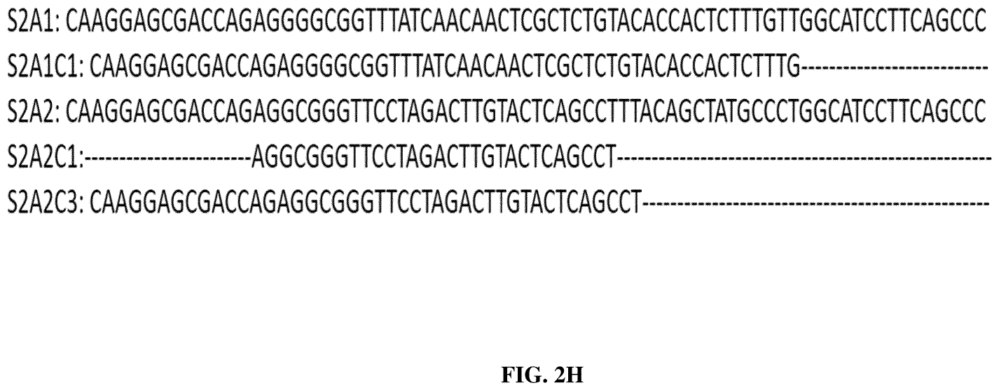

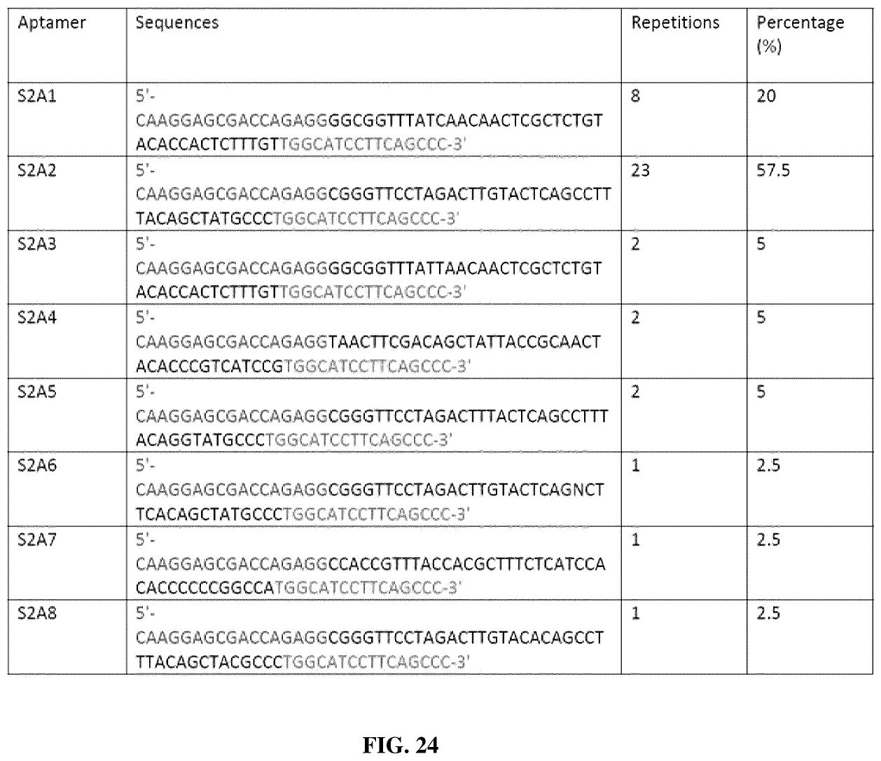

Fifteen cycles of selection against the SARS-CoV-2 WT S2 protein were performed (FIG. 2A) using an ssDNA library encompassing a 40-nt randomized region (FIG. 24). For the initial selection cycle, 3 nmol of the DNA library were used, which provided wide sequence diversity with ˜10¹⁵ unique DNA molecules. The His-tagged S2 protein was immobilized on nickel nitrilotriacetic acid (Ni-NTA) magnetic beads and incubated with the DNA library in each selection cycle. After washing by selection buffer, DNA bound to the beads was eluted and subjected to PCR amplification. A counterselection step, starting from the 3^{rd} round, was introduced in each alternative round to remove unspecific DNA binders, using unembellished Ni-NTA beads (FIG. 2A). Iterative cycles of selection while reducing the amount of protein and DNA, as well as the incubation time, yielded the enrichment of a specific pool against the S2 protein (FIGS. 2C, 2E). The details of each selection cycle are listed in Table 2. To monitor and assess the enrichment of the specific binding between the DNA library and the S2 protein over the selection rounds, 6-FAM-labeled ssDNA were generated from different SELEX rounds (from the 10^{th} to the 15^{th} rounds) using a 6-FAM-labeled reverse primer. As shown in FIG. 2B, these libraries were incubated with the S2 protein, which was immobilized on magnetic streptavidin beads. After washing, the 6-FAM-labeled libraries were eluted, and their fluorescence intensities were recorded using a plate reader. As shown in FIG. 2C, the fluorescence signal significantly increased from the 10^{th} round to the 11^{th} round and consistently increased to its maximum at the 15^{th} round. In addition, the specific enrichment was also determined using fluorescence microscopy (FIG. 2D). The His-tagged S2 protein was immobilized on a Ni-NTA resin, and incubated with 6-FAM labeled ssDNAs. After washing, the resin was subjected to fluorescence imaging. As shown in FIG. 2E, the pool from the 15^{th} round displayed a much stronger fluorescent signal than that from the 10^{th} or 11^{th} round. Furthermore, the 6-FAM-labeled ssDNA from the 15^{th} round did not bind to the control His-tagged protein immobilized on a Ni-NTA resin (FIG. 2E, right). This indicates that there is high binding specificity of the ssDNA library from the 15^{th} round toward the S2 protein. The above data indicate that after 15 rounds of SELEX, a useful aptamer candidate pool with good binding ability against the S2 protein was obtained. For this reason, the enriched aptamer candidate pool from the 15^{th} round was cloned using a TOPO TA Cloning® Kit. The product of recombination was used to transform E. coli component cells, and random colonies were sequenced. A total of 8 aptamer candidates, whose sequences are given in FIG. 24, were obtained. Two major groups of aptamer candidates were observed, in which the sequence of S2A1 (5′-CAAGGAGCGACCAGAGGGGCGGTTTATCAACAAC TCGCTCTGTACACCACTCTTTGTTGGCATCCTTCAGC CC-3′ (SEQ ID NO: 4)) occupies 20% of the whole sequencing data, and the sequence of S2A2 (5′-CAAGGAGCGACCAGAGGCGGG TTCCTAGACTTGTACTCAGCCTTTACAGCTATGCCCTGGCATCCTTCAGCCC-3′ (SEQ ID NO: 5)) occupies 57.5%.

### Characterization of Binding Affinities for Anti-S2 Aptamers

The aptamer candidates S2A1 and S2A2 were synthesized and received from the Integrated DNA Technology (IDT). Their sequence and secondary structure information are provided in FIGS. 2G, 2H. To reduce the cost of synthesis and other complications that may arise using a longer nucleotide chain, both original aptamers were shortened based on their common sequence and predicted structural analysis using NUPACK, by removing the redundant nucleotides from the 3′, 5′, or both ends. Three truncated aptamers, S2A1C1, S2A2C1, and S2A2C3, were generated (FIGS. 2G, 2H). The equilibrium dissociation constants (K_{d}) of all candidates were measured via a streptavidin bead-based fluorescence assay, in which the response of the fluorescence emission intensity was measured as a function of various concentrations of the 6-FAM-labeled aptamer (3, 10, 30, 100, 300, 1000 nM) bound with 5 pmol of the S2 protein (FIG. 2B). As shown in FIG. 2F, S2A1 and S2A2 bind the S2 protein with similar binding affinities, K_{d}=49.7±3.2 and 44.2±6.6 nM, respectively. It was found that three truncated aptamers, namely, S2A1C1, S2A2C1, and S2A2C3, show a slightly better binding affinity towards the S2 protein than their parent aptamers (FIG. 2F). Among all anti-S2 aptamers, S2A2C1 has the shortest sequence, with a hairpin structure, and also has the best binding affinity towards the S2 protein (K_{d}=35±4.3 nM).

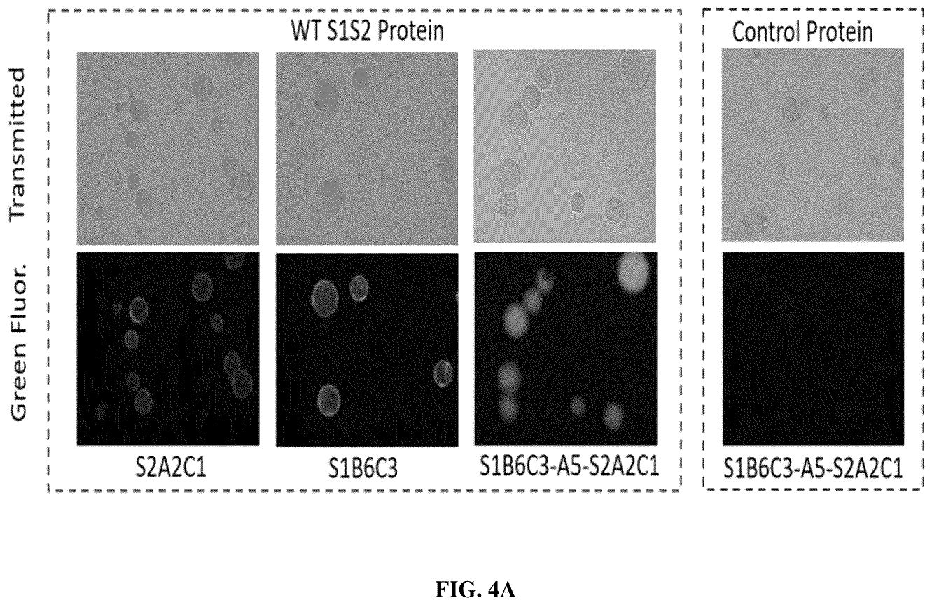

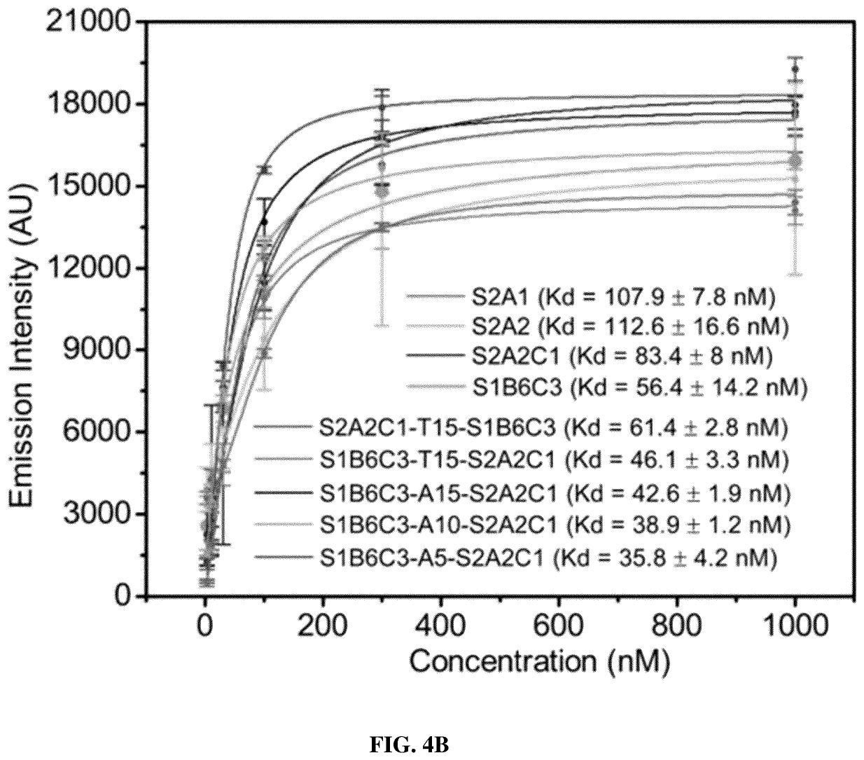

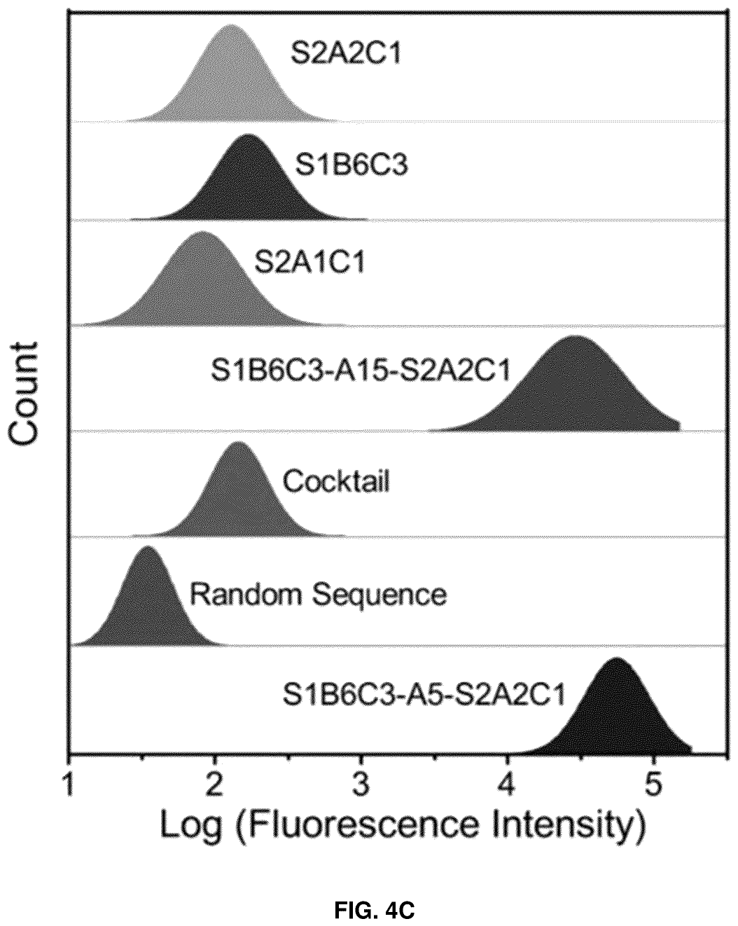

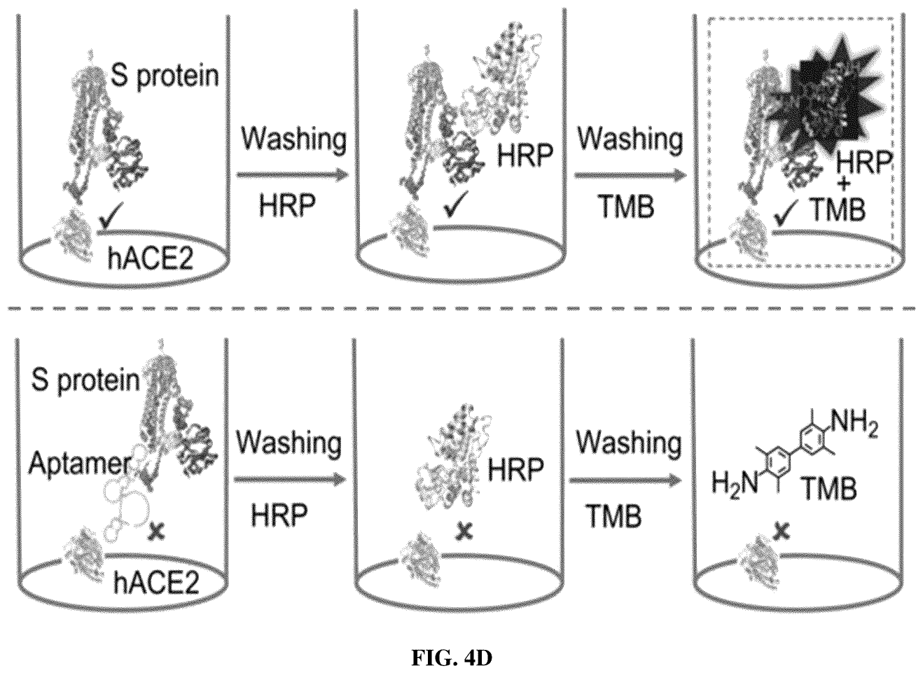

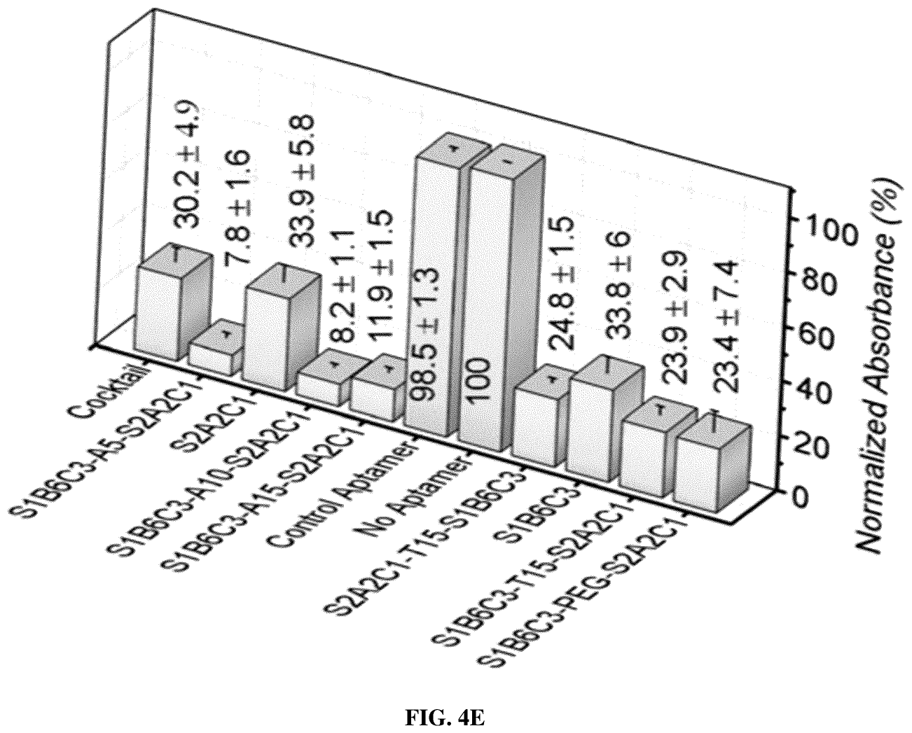

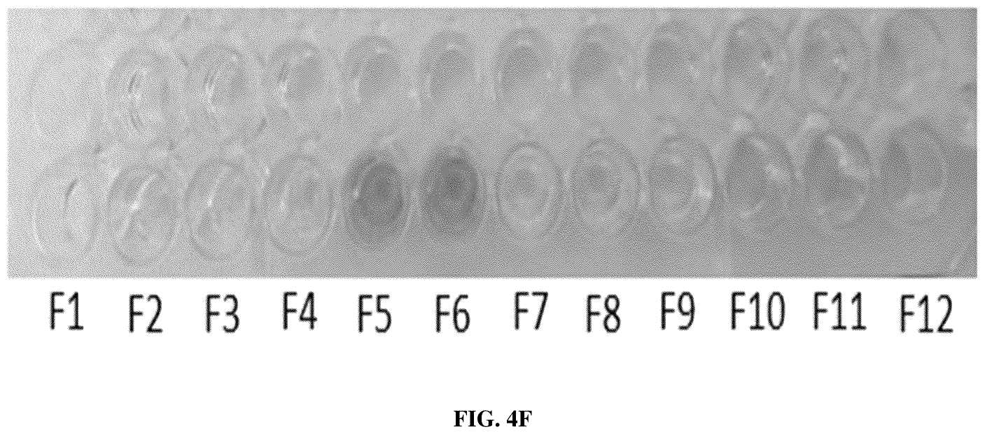

Next, whether these anti-S2 aptamers can still recognize the S2 segment in the whole S1S2 extracellular domain was evaluated, since the S2 domain may undergo conformational changes when it is co-expressed with the S1 domain. To answer this question, the K_{d} values for S2A1, S2A2, and S2A2C1 against the S1S2 protein were measured. As shown in FIG. 4B, both original aptamers and the optimized S2A2C1 were observed to bind well to S1S2, with moderately higher binding K_{d} values compared to S2. Since the S2A2C1 aptamer showed the best binding to the S1S2 or S2 proteins, it was subsequently used to study the binding specificity. The data show that S2A2C1 can recognize either the S2 protein or the S2 domain in the spike protein.

### Characterization of the Binding Specificity of S2A2C1

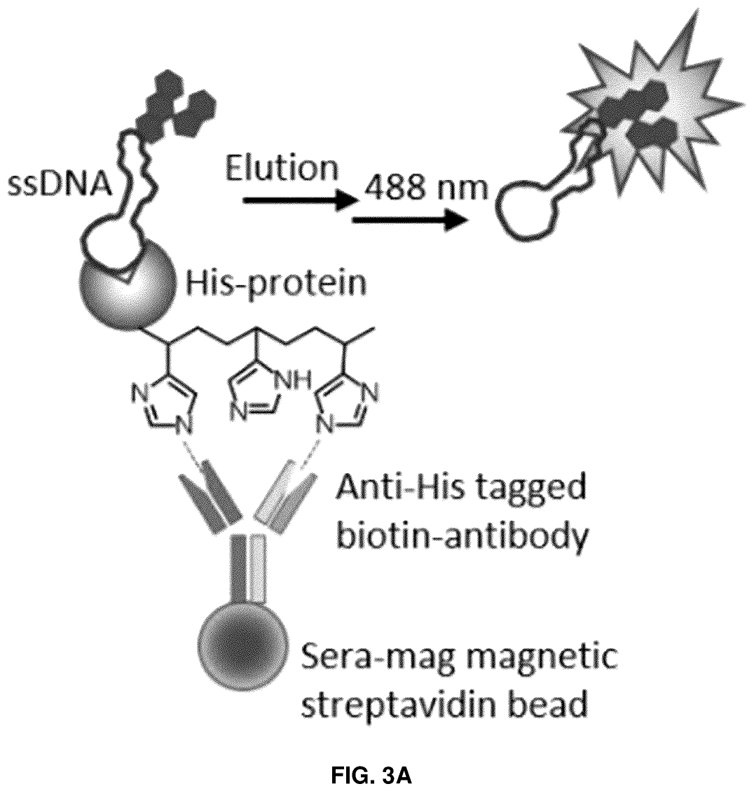

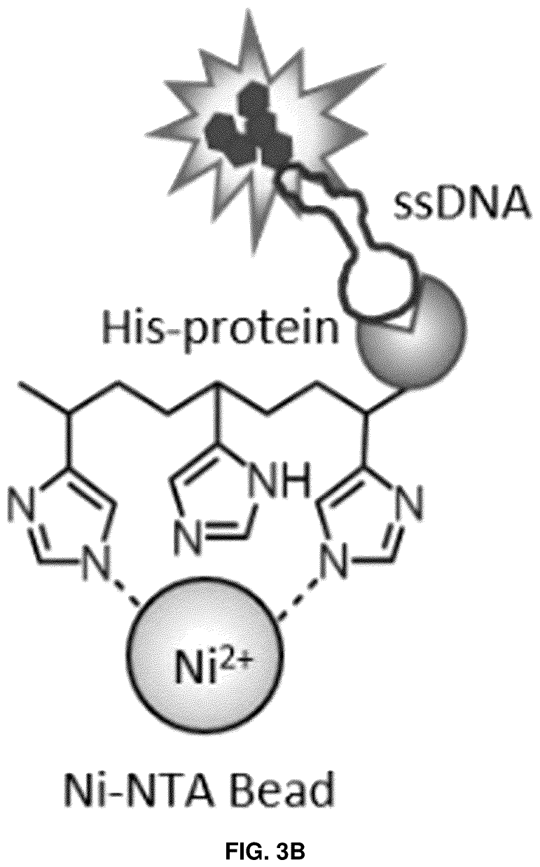

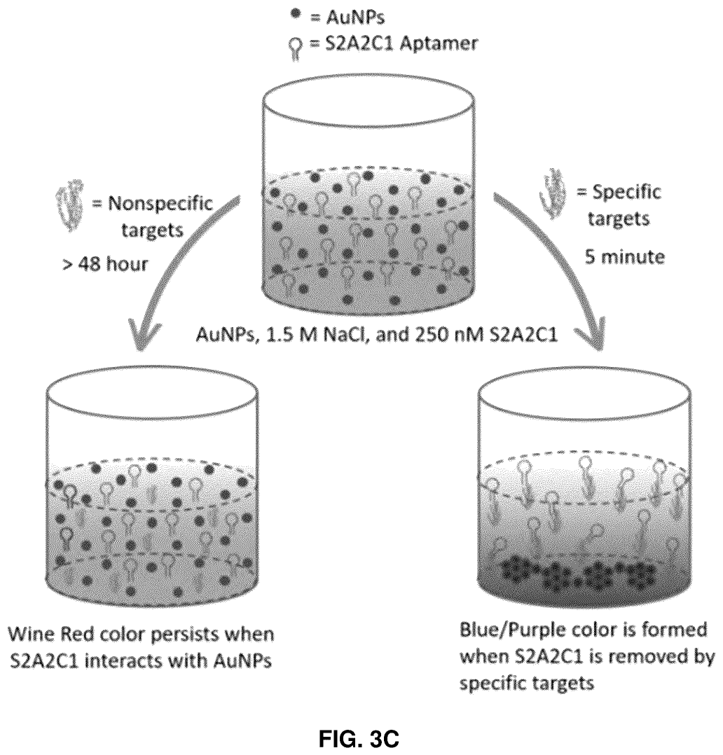

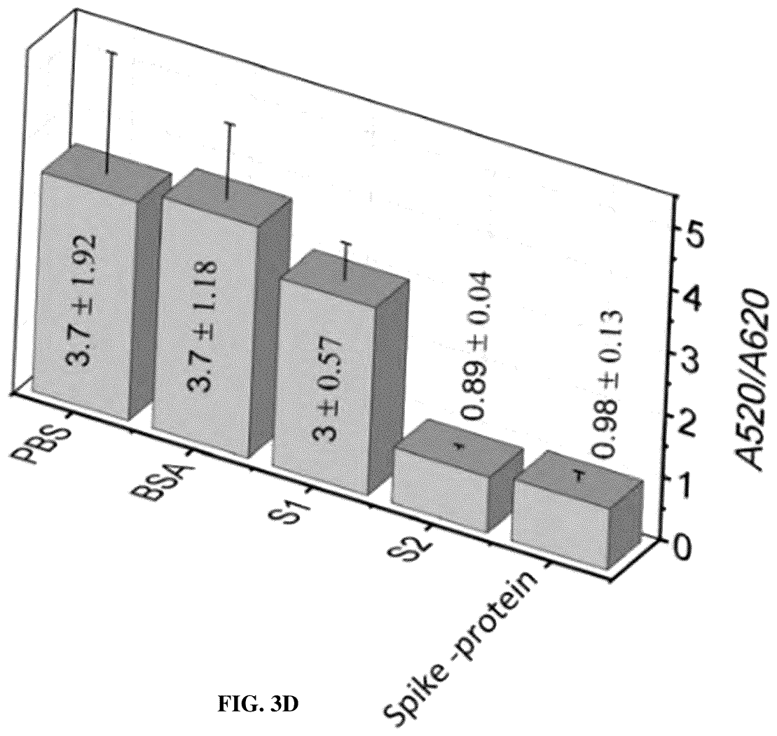

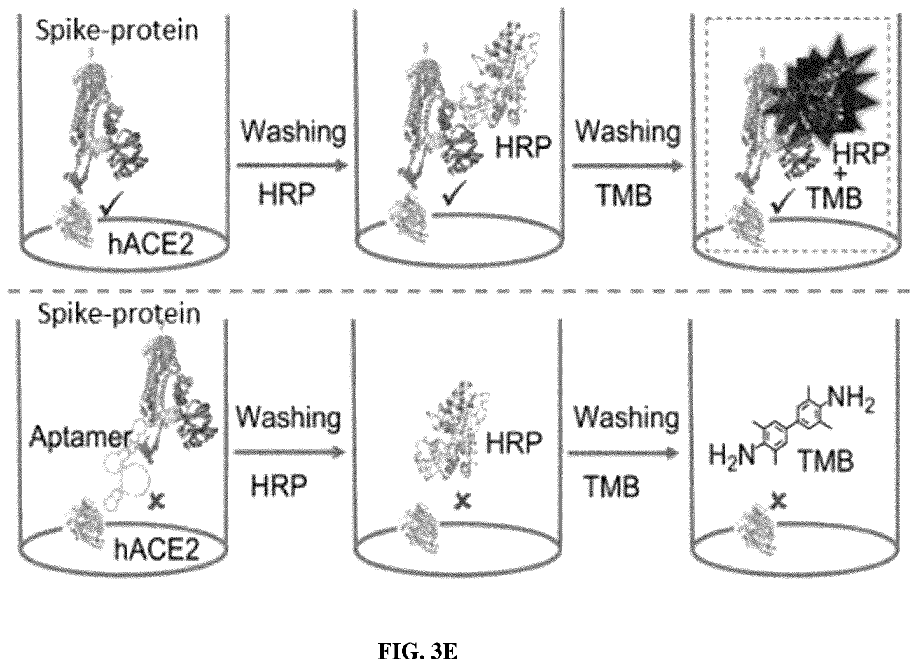

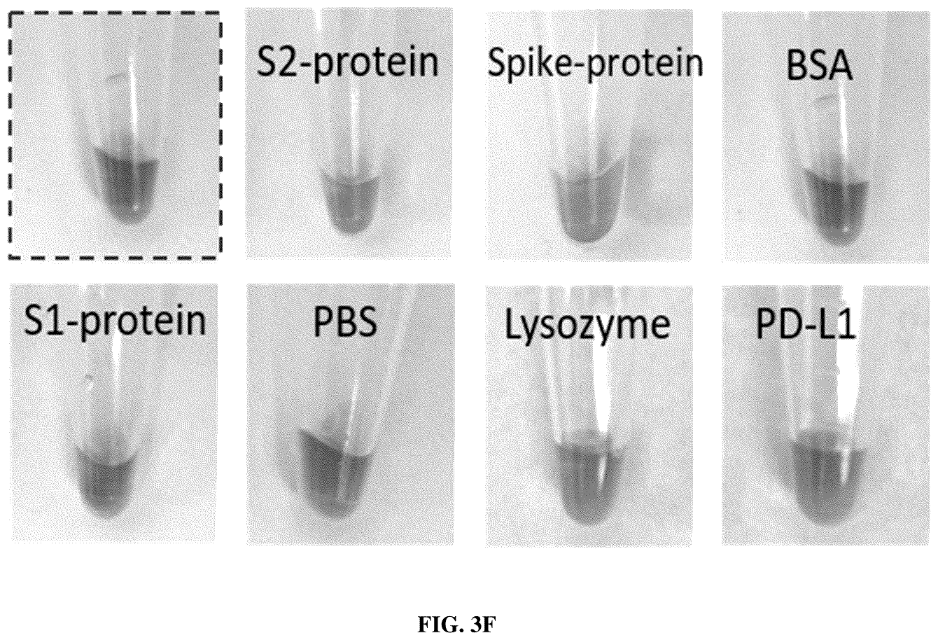

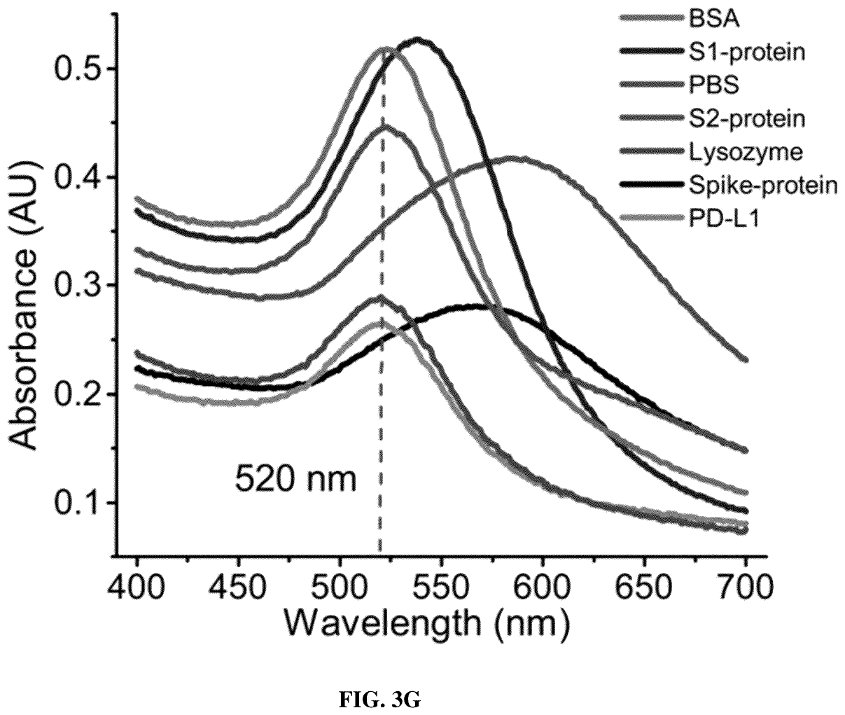

To validate the binding specificity of the most desirable truncated aptamer S2A2C1 to S2-protein, the well-established gold nanoparticles (AuNPs) based colorimetric assay was performed. AuNPs based colorimetric assay was used, confirming that the S2A2C1 aptamer does not bind the S1-protein and only binds to the S2-protein or S2 domain in the spike-protein. The AuNPs colorimetric assay, as demonstrated in FIG. 3C, is based on the function of negatively charged DNA molecules to prevent the AuNPs aggregation. The detailed mechanism and kinetics of the interaction between aptamer and AuNP that prevents AuNP from aggregation have been extensively characterized. However, when a specific target of the DNA aptamer in AuNPs colloids is added, the aggregation starts, which changes the color of AuNPs from wine-red to blue or purple. In the presence of the S2A2C1 aptamer, the AuNPs persist red color (FIG. 3F, encircled with blue dots). Briefly, in this experiment, when the S2- or spike-protein was added into the colloids of AuNPs, NaCl, and the S2A2C1 aptamer, the protein was preferably bound to the S2A2C1 aptamer, activating the AuNPs aggregation. The wine-red color of the AuNPs colloids was intact for more than 48 hours in the presence of 1.5 M NaCl and 250 nM S2A2C1 aptamer. However, it dramatically changed into blue or purple within 5 minutes of adding 250 nM of the S2- or spike-protein (FIGS. 3F, 3C). Besides visual color change, UV-Vis measurement was also employed to measure the effect of various targets on the AuNPs/S2A2C1/NaCl colloid (FIG. 3G). The S2- or spike-protein caused the significant redshift to the characteristic peak of AuNPs colloids located at 520 nm. However, nonspecific targets such as the S1-protein, lysozyme, or PD-L1 did not cause the obvious redshift to the characteristic peak (FIG. 3G). This experimental evidence shows that S2A2C1 specifically binds to the S2-domain regardless of whether it is either isolated or present in the whole spike-protein. In addition, the ratios of A520/A620 can be used to define the specificity of the interaction between the proteins and the aptamer. It should be mentioned that the A520/A620 ratio is not calibrated to the percent bound. As shown in FIG. 3D, the A520/A620 values are greater than 3 for S1, BSA, and buffer, indicating that they have negligible binding to the S2A2C1 aptamer. Where the A520/A620 values are smaller than 1 for S2- and spike-protein, it refers to both being the specific binders to S2A2C1. In summary, the aptamer S2A2C1 can specifically bind to the S2-protein, but not to the S1-protein (RBD).

### Anti-S2 Aptamer S2A2C1 Inhibits the S Protein/hACE2 Interaction Via an RBD Independent Approach

The aptamer S2A2C1 was obtained by SELEX on the S2 protein, and it was also confirmed that it specifically binds to S2, but not to S1. A fusion aptamer composed of S2A2C1, a linker, and an anti-S1 aptamer S1B6C3 was constructed. The S1B6C3 aptamer (5′-CGCAGCACCCAAGAACAAGGACT GCTTAGGATTGCGATA-GGTTCGG-3′ (SEQ ID NO: 6) was selected against RBD and can efficiently block the S protein/hACE2 interaction. Before constructing the fusion aptamer, the binding affinity of S1B6C3 toward the S1S2 protein was measured, with K_{d}=56.4±14.2 nM, which is lower than that of S2A2C1 on S1S2, K_{d}=83.4±8 nM, as shown in FIG. 4B. Flow cytometry measurements also demonstrate that the 6-FAM-labelled S2A2C1 and S1S2B6 virtually have similar binding efficacies towards the WT spike protein (FIG. 4C). Next, an Enzyme-Linked Immunosorbent Assay (ELISA) was used to examine how both aptamers inhibit the S1S2/hACE2 interaction. Briefly, the plate wells were coated with non-His-tagged hACE2 and then blocked by BSA to avoid any nonspecific binding. After washing, the His-tagged spike protein was added which can be recognized by an HRP anti-His-tag antibody with the substrate TMB. This gives a strong absorbance at 450 nm after adding acid (FIG. 4F, well F6). The normalized absorbance is proportional to the amount of target protein present. In the presence of an aptamer, such as S1B6C3, which can effectively neutralize S1S2 and prevent its binding to hACE2, the His-tagged S1S2 will be removed during washing. Consequently, HRP cannot generate correspondingly strong signals. As shown in FIGS. 4E, 4F (well F8), S1B6C3 inhibited 66% of S1S2 to bind to hACE2. Surprisingly and interestingly, S2A2C1, as an anti-S2 aptamer, also inhibited 66% of S1S2 binding to hACE2, as shown in FIGS. 4E, 4F (well F2). At present, this RBD independent mechanism is unknown. Without wishing to be bound by theory, it is believed that once S2A2C1 binds to the S2 domain, it induces an allosteric effect on the S1 domain, affecting its binding with hACE2. The control aptamer cannot prevent the S1S2/hACE2 interaction (FIGS. 4E, 4F, well F5), indicating both S1B6C3 and S2A2C1 specifically block S1S2.

### Design of Fusion Aptamers and Characterization of Their Binding Affinity and Inhibition Efficacy Against WT S1S2

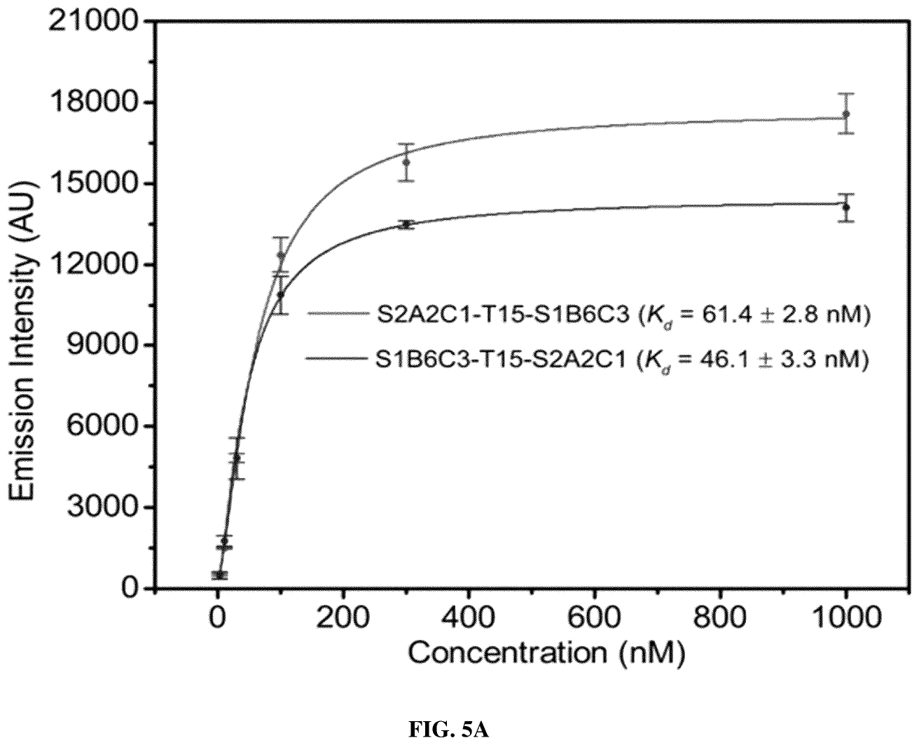

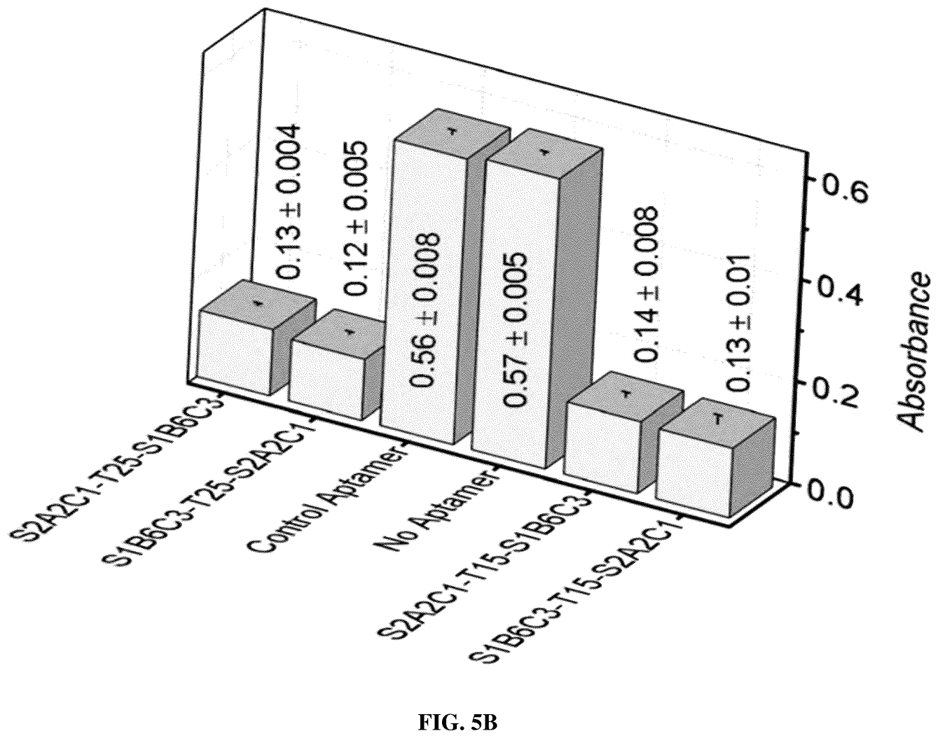

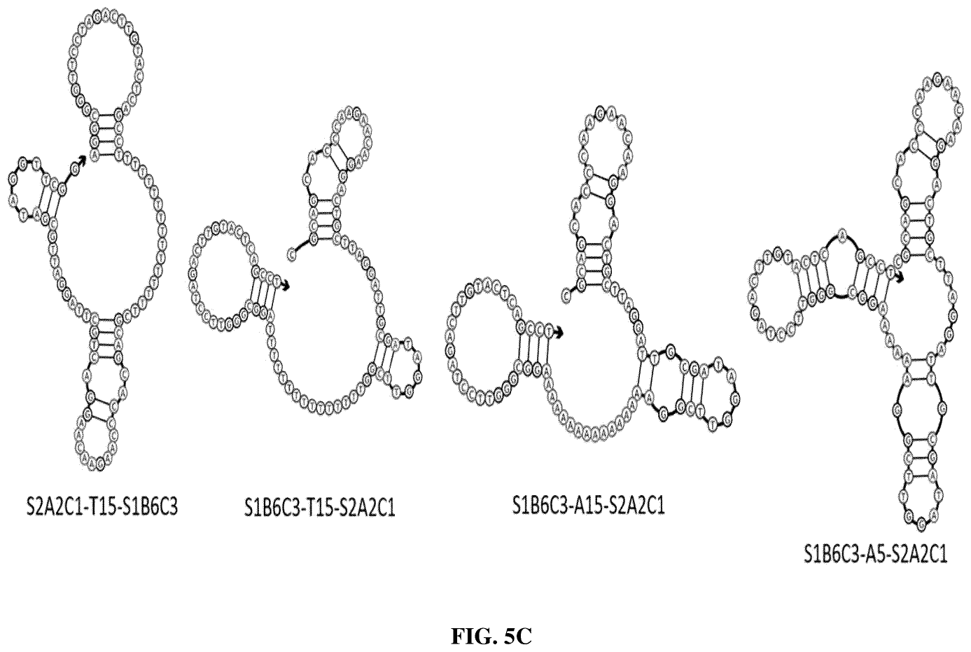

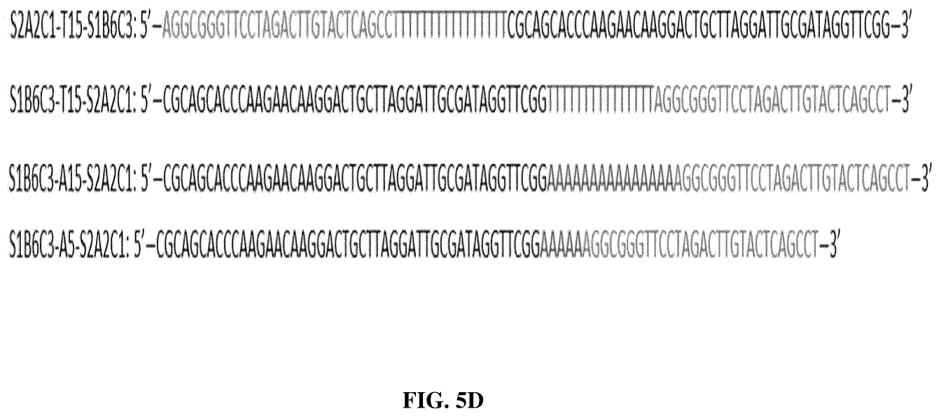

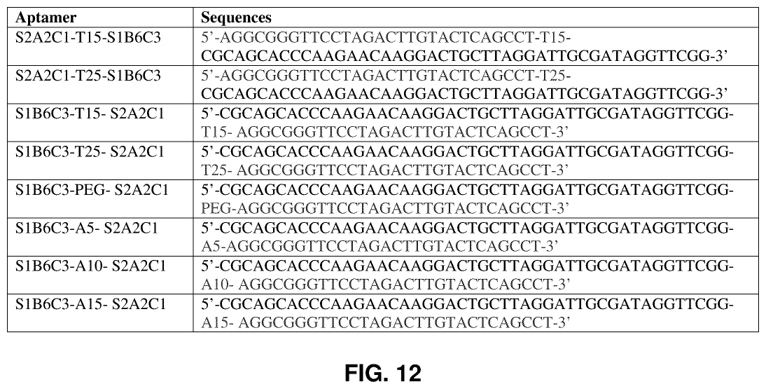

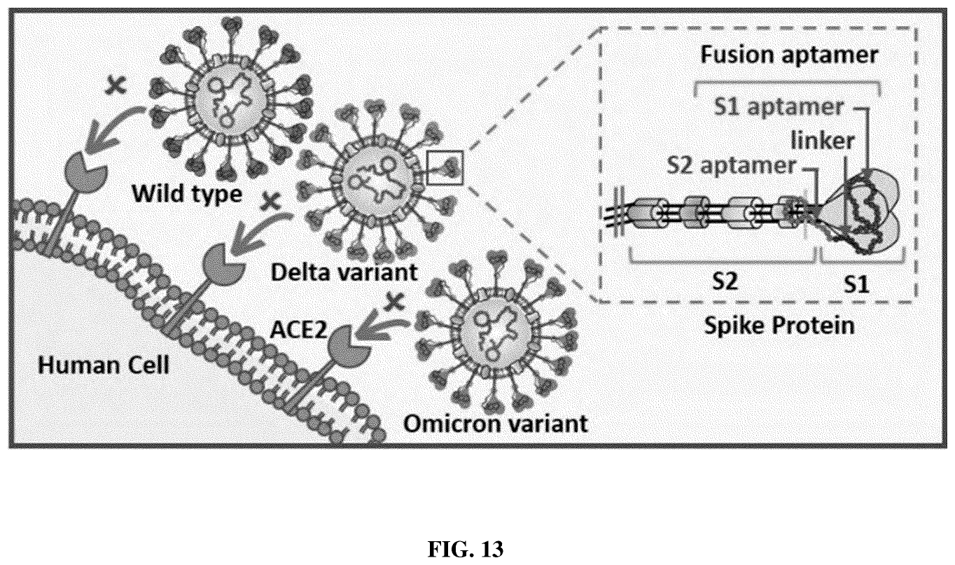

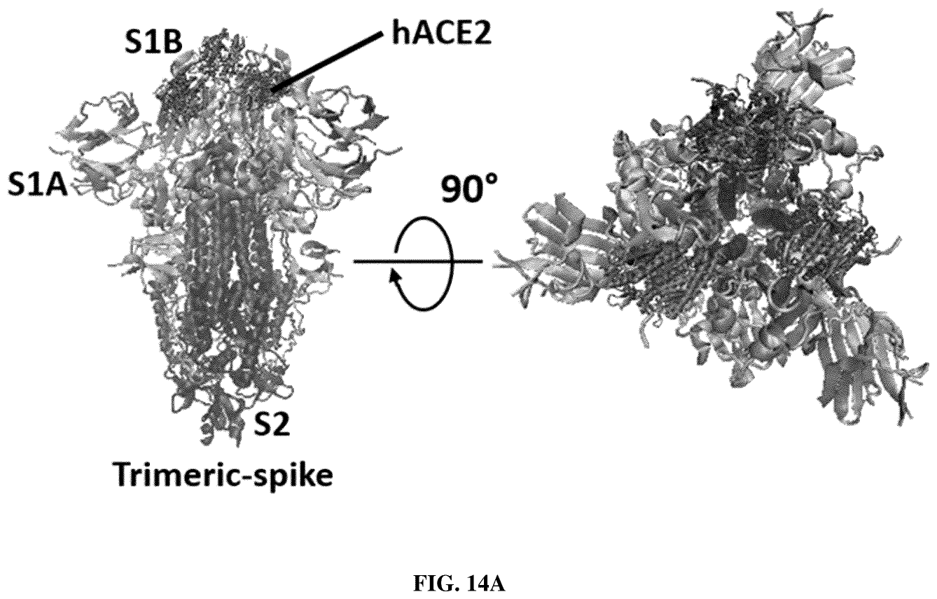

Next, fusion aptamers were constructed using aptamers S1B6C3 and S2A2C1 with various linkers such as polyethylene glycol (PEG), T25 (SEQ ID NO: 29), T15 (SEQ ID NO: 28), A15 (SEQ ID NO: 30), A10 (SEQ ID NO: 31), and A5 (Table 3, FIG. 12). These fusion probes can bind to the S1S2 protein at two sites, offering improved binding affinity and inhibition efficacy. First, four fusion aptamers, S2A2C1-T25-S1B6C3, S2A2C1-T15-S1B6C3, S1B6C3-T25-S2A2C1, and S1B6C3-T15-S2A2C1 were generated, all having a long polyT linker (25- (SEQ ID NO: 29) or 15-mer (SEQ ID NO: 28)). However, the constituent mono aptamers reside in a different orientation, since this direction may affect the function of the corresponding fusion aptamer. The inhibition efficacies were first measured for the four fusion aptamers, and it was observed that all of them have very close inhibition efficacies to block the S1S2/hACE2 interaction (FIG. 5B). This indicates that there is almost no difference in using T25 (SEQ ID NO: 29) vs. T15 (SEQ ID NO: 28) linkers. To save the cost of synthesis, it was decided to use T15 (SEQ ID NO: 28) as the linker and measure the binding affinities of aptamers S2A2C1-T15-S1B6C3 and S1B6C3-T15-S2A2C1, in order to decide which orientation is better. As shown in FIG. 4B, S1B6C3-T15-S2A2C1 (K_{d}=46.1±3.3 nM) is superior to S2A2C1-T15-S1B6C3 (K_{d}=61.4±2.8 nM) in recognizing the WT S1S2 protein. Therefore, S1B6C3 was set at the 5′ end for subsequent fusion aptamer design.

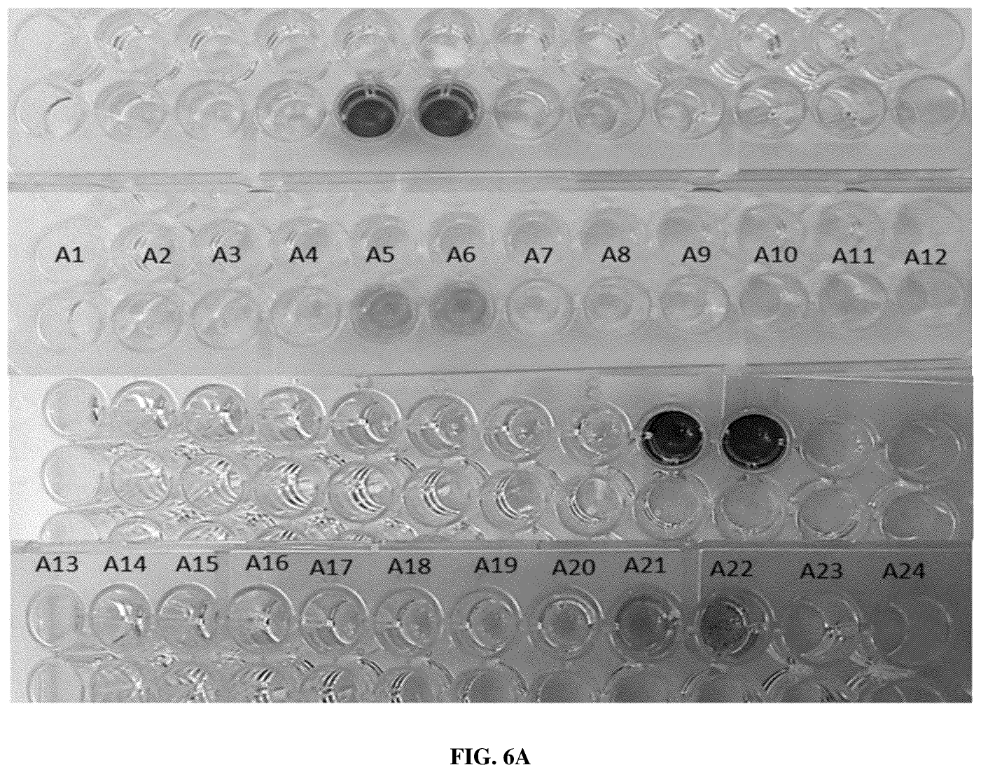

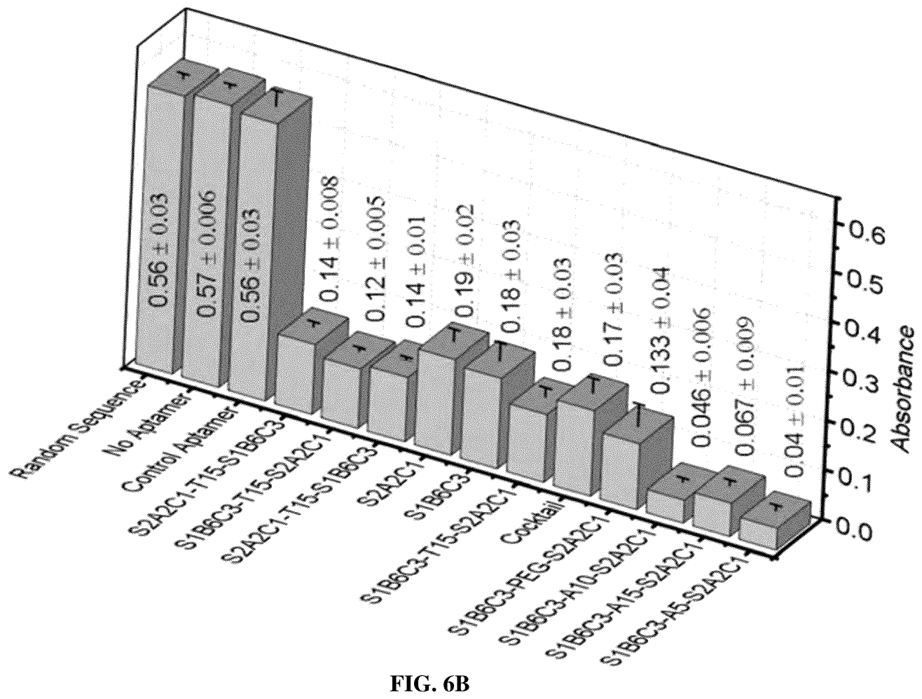

Next, the change of the linker T15 (SEQ ID NO: 28) to A15 (SEQ ID NO: 30) or a polyethylene glycol (PEG) linker, which has a similar length compared with the former two linkers, was tested. Although this change does not affect too much of the predicted secondary structures of corresponding fusion aptamers (FIG. 5C), it may still influence their functions. As shown in FIG. 4E, S1B6C3-A15-S2A2C1 has an obviously better inhibition efficacy (with its presence, only 12% of the S1S2 protein can bind to hACE2) than that of S1B6C3-T15-S2A2C1 (24%) or S1B6C3-PEG-S2A2C1 (23%), indicating a poly A linker should continue to be used. Finally, it was attempted to further shorten the linker length, thoroughly measuring the improvement in binding affinity and inhibition efficacy by comparing A15 (SEQ ID NO: 30), A10 (SEQ ID NO: 31), and A5 linkers. It was concluded that S1B6C3-A5-S2A2C1 is the most desirable fusion aptamer (FIGS. 4B, 4C, 4E, 6A-6B), which has 2.3 folds better binding affinity (K_{d}=35.8±4.2 nM) in comparison with both constituent mono aptamers.

S1B6C3-A5-S2A2C1 was also compared with the aptamer cocktail (mixture of S1B6C3 and S2A2C1 in a 1:1 molar ratio). The former is a single DNA molecule, and the latter contains two DNA molecules. In the presence of S1B6C3-A5-S2A1C1, only 8% WT S1S2 can bind to hACE2, but with the same molar concentration of both mono aptamers in the cocktail, more than 30% WT S1S2 can still bind to hACE2 (FIGS. 4E, 4F, 6A-6B). These results show that the fusion aptamer, as a single molecular probe, enhances inhibition efficacy in comparison to the cocktail, to block S1S2 binding to hACE2. All of the above data indicate a single molecular fusion aptamer is far superior to mono aptamers in binding to WT S1S2 as well as inhibiting the S1S2/hACE2 interactions. The refined fusion aptamer S1B6C3-A5-S2A2C1 was used for subsequent experiments.

### Aptamers Show Good Binding and Inhibition Efficacy to the Delta and Omicron S1S2 Proteins

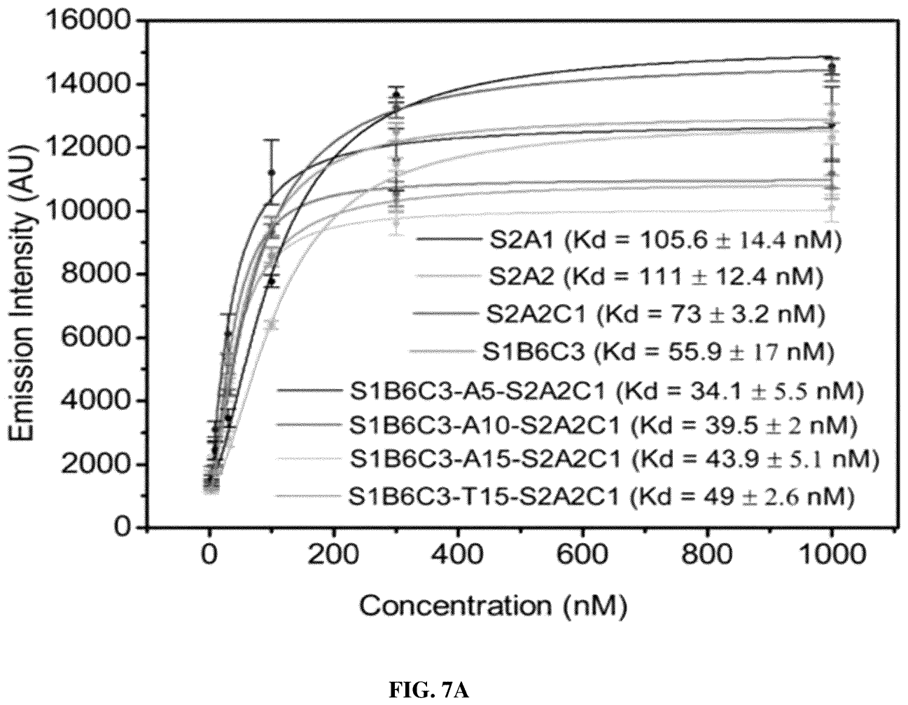

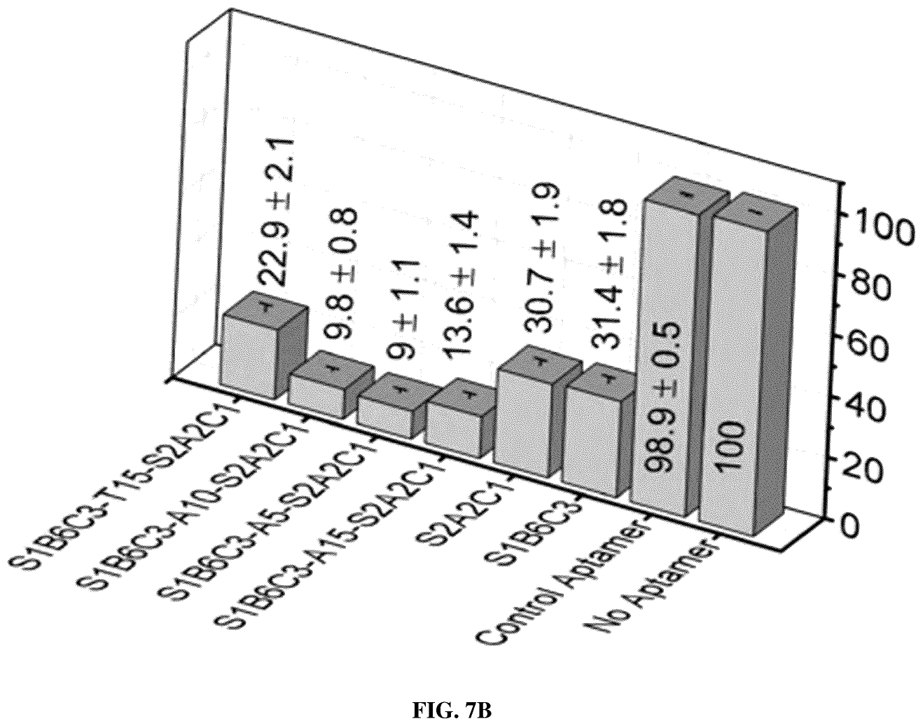

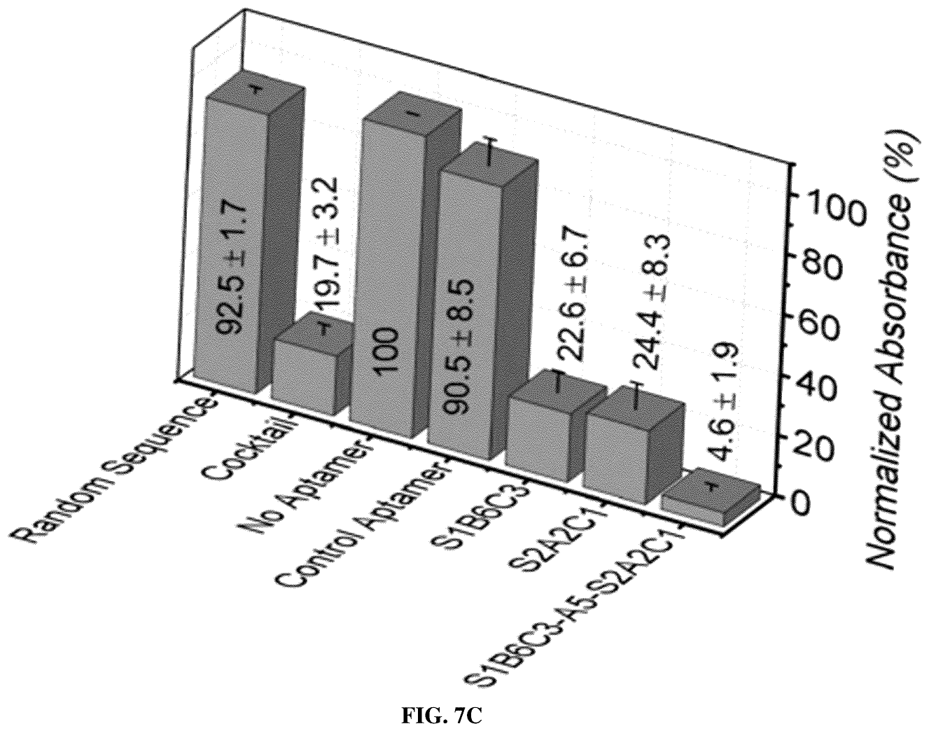

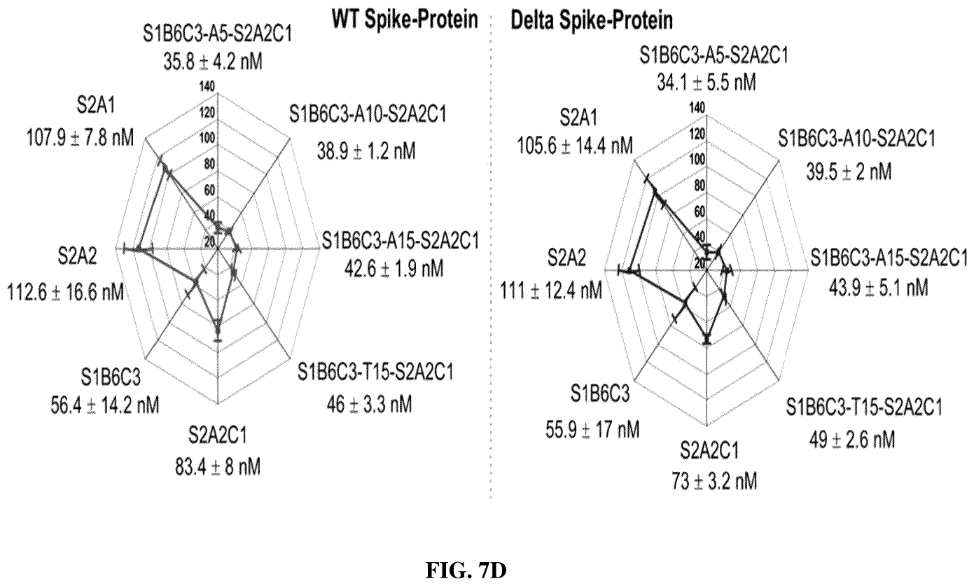

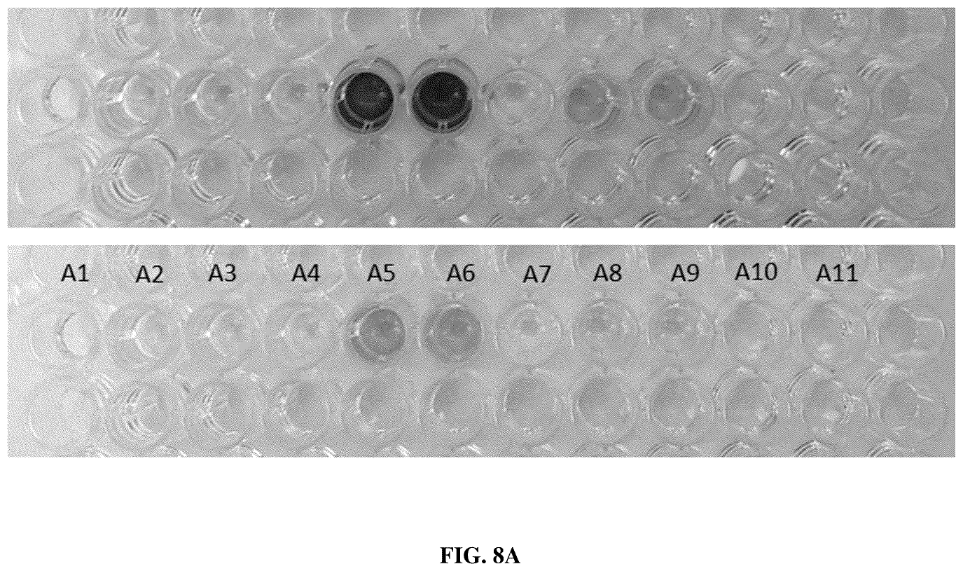

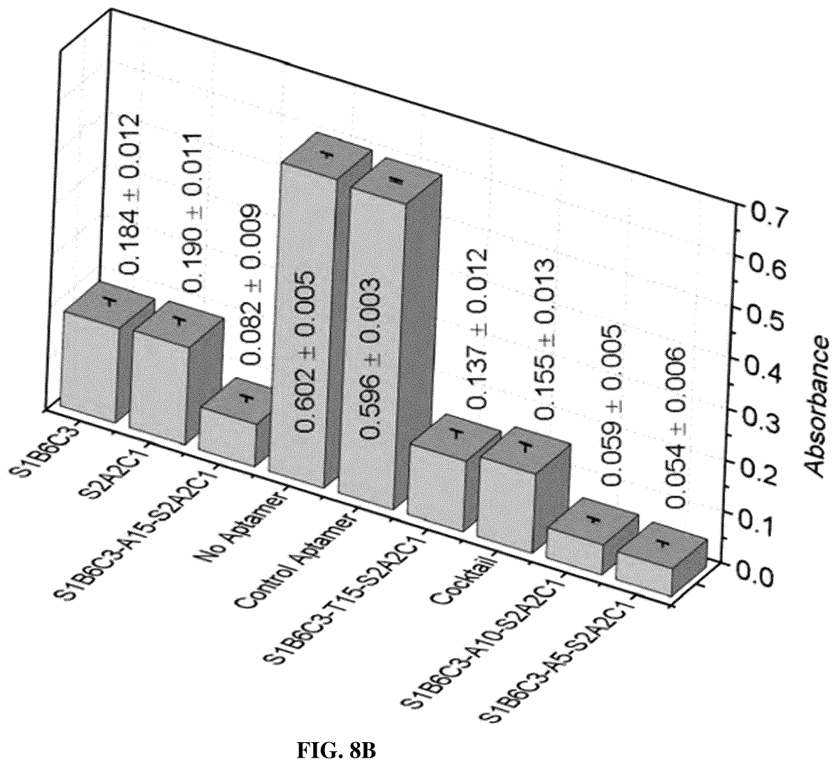

Based on CDC reports, the Delta and Omicron variants have a few mutations in the gene encoding the SARS-CoV-2 S protein. Most mutations are located on the S1 protein, although some are on the S2 protein, indicating that S2 is highly conserved. To examine whether S2A2C1 and the fusion aptamers still can inhibit these mutated S1S2 proteins recognizing hACE2, experiments to determine the binding affinity and inhibition efficacy of mono, cocktail, and the fusion aptamers against the Delta variant S1S2 protein were first performed. FIG. 7A shows the K_{d} values of various aptamers against Delta S1S2 protein. In addition to binding affinity, S1B6C3-A5-S2A2C1 displays the best inhibition efficacy against the Delta and Omicron spike protein in comparison to other fusion aptamers (FIGS. 7B, 7C). As shown in FIG. 7D, the K_{d} values of various aptamers were also compared with the WT and Delta S1S2 proteins, and it was observed that all tested aptamers have very similar binding affinities on both WT and Delta S1S2 proteins. This indicates that mutated amino acids on the Delta S1S2 protein do not influence the binding between these aptamers and the S1S2 protein. Therefore, these aptamers will also be able to efficiently block the Delta S1S2 protein/hACE2 interaction. This is supported by the data shown in FIGS. 7B, 8A-8B, 9A-9B. The results show that in the presence of S2A2C1, 31% Delta S1S2 protein can bind to hACE2, and this value decreases to 9% when S1B6C3-A5-S2A2C1 was used.

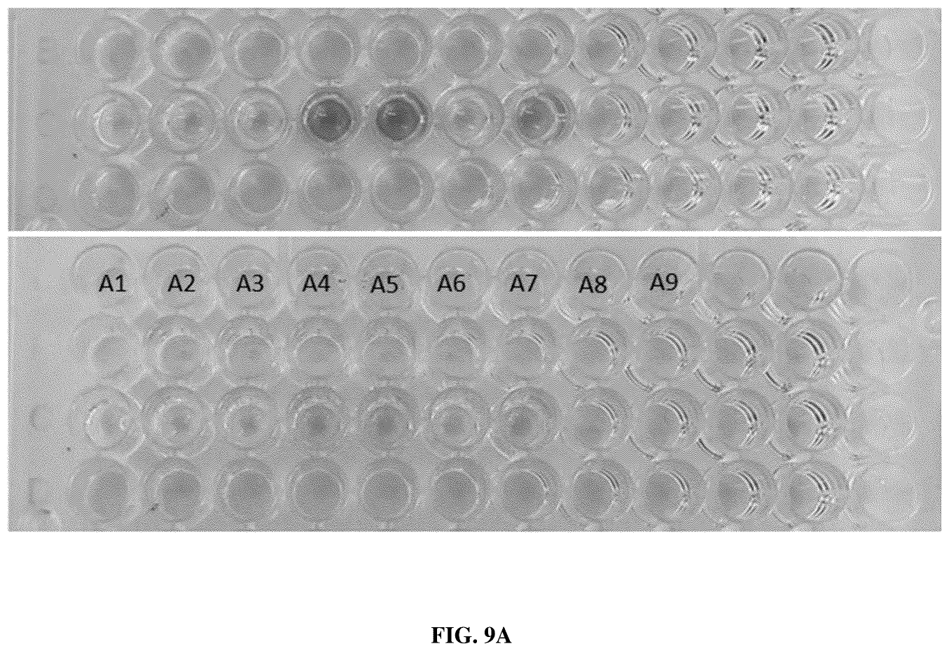

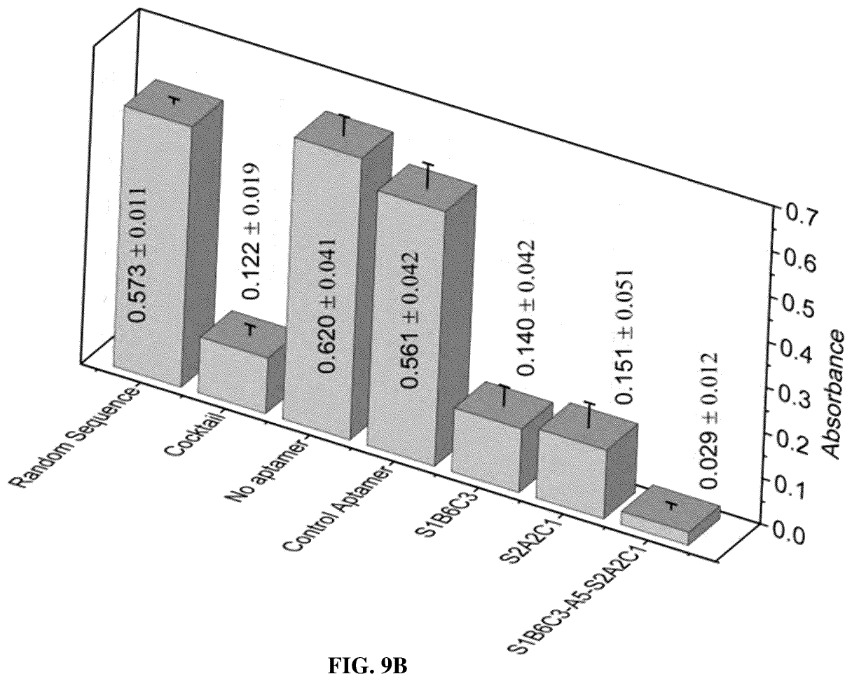

Finally, the Omicron S1S2 protein was also examined using the aptamers. As shown in FIGS. 7D, 9A-9B, in the presence of S1B6C3-A5-S2A2C1, only 5% Omicron S1S2 can still bind to hACE2. For S2A2C1 the residual binding is 24%, and for the aptamer cocktail, it is 20%. From the results, it can be concluded that S2A2C1 and the fusion aptamer S1B6C3-A5-S2A2C1 bind to both Delta and Omicron S1S2 proteins irrespective of these S1S2 proteins' mutated residues. The variants do not cause large conformational changes in the SARS-CoV-2 S protein, which may be the reason the anti-WT aptamers can still neutralize the Delta and Omicron S1S2 proteins regardless of the mutant residues. Results from overall binding affinity and inhibition assays demonstrate that the fusion aptamer S1B6C3-A5-S2A2C1 is a desirable tool that may prevent SARS-CoV-2 and its variants from infecting cells.

### Flow Cytometry Approach to Determine the Binding Affinity of the S1B6C3-A5-S2A2C1 Aptamer Against the WT, Delta, and Omicron Spike Proteins

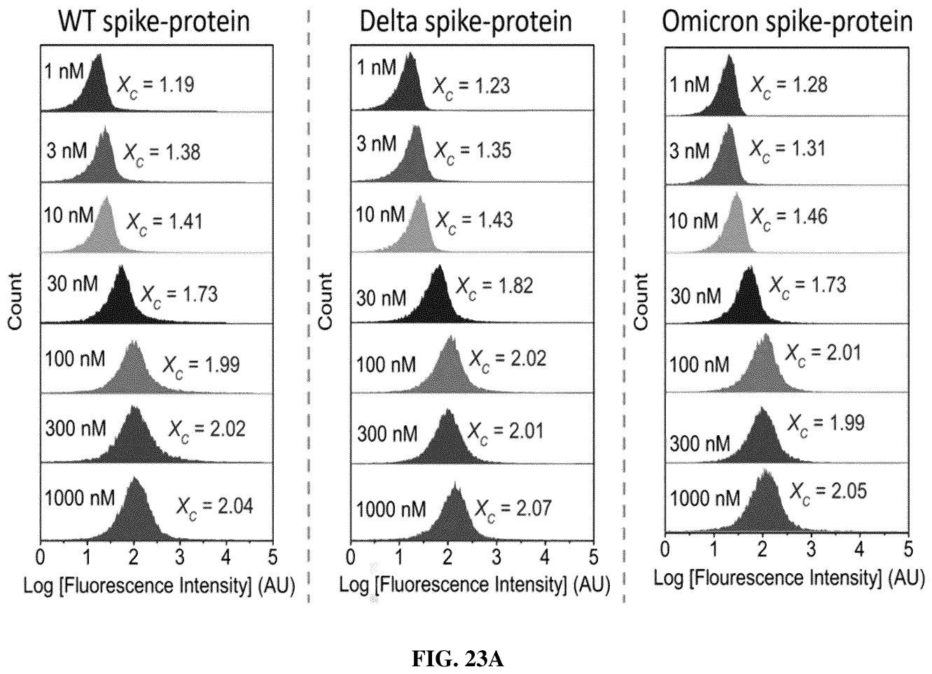

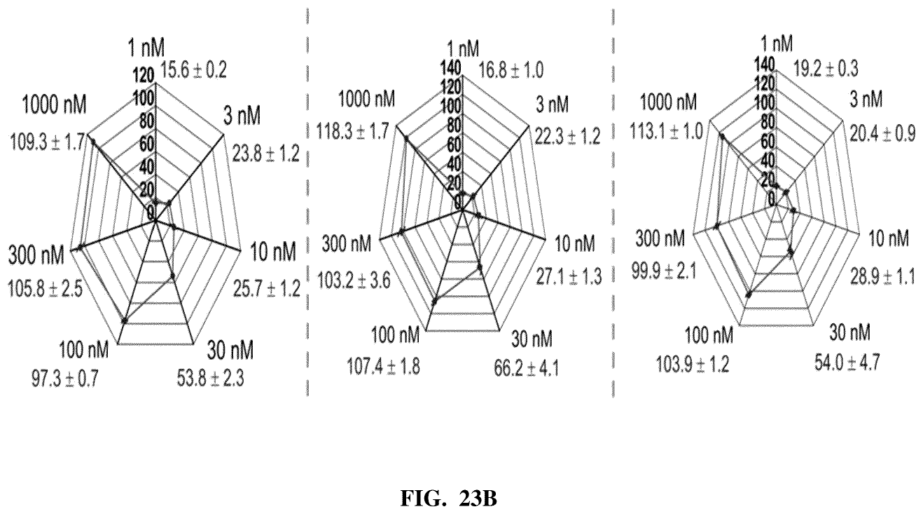

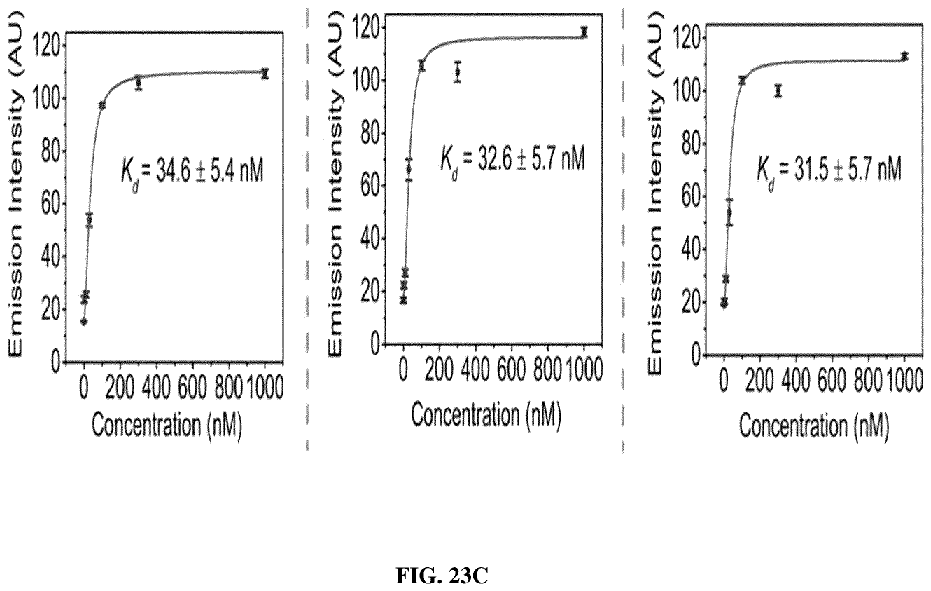

In addition to the K_{d} determination using a clariostar microplate reader, the flow cytometer was also used to measure the K_{d} values of the fusion aptamer S1B6C3-A5-S2A2C1 against the WT, Delta, and Omicron spike-proteins (FIGS. 23A-23C). To calculate the K_{d} values, three trials of the flow cytometer measurements were integrated for all seven concentrations (1, 3, 10, 30, 100, 300, and 1000 nM) of the S1B6C3-A5-S2A2C1 aptamer treated against various spike-proteins (WT, Delta, and Omicron). The K_{d} values of S1B6C3-A5-S2A2C1 aptamer against WT, Delta, and Omicron determined by flow cytometer are 36.4±5.4, 32.6±5.7, and 31.5±5.7 nM, respectively, which are consistent with those determined using the Clariostar microplate reader. The comparison of the K_{d} values measured by the clariostar plate reader and flow cytometer is summarized in following Table 8.

TABLE 8
Comparison of the binding affinities measured by microplate
reader and flow cytometry approaches of S1B6C3-A5-S2A2C1
aptamers against WT and Delta spike-protein

| | K_{d} from | |
|---|---|---|
| | clariostar | K_{d} from |
| | microplate | flow |
| Interactions | reader | cytometer |
| S1B6C3-A5- S2A2C1 against | 35.8 ± 4.2 nM | 34.6 ± 5.4 nM |
| WT spike-protein | | |
| S1B6C3-A5- S2A2C1 against | 34.1 ± 5.5 nM | 32.6 ± 5.7 nM |
| Delta spike-protein | | |

### Conclusion

Anti-S2 aptamers have been discovered by exploiting DNA SELEX methods on the WT S2 protein. Redundant nucleotides from the original aptamers were removed to obtain truncated aptamers, with good binding specificity and affinity toward the S2 protein domain. As the most desirable anti-S2 aptamer, S2A2C1 showed the maximum binding affinity, and more importantly, it also showed the virtuous efficacy to neutralize the WT S1S2 protein/hACE2 interaction, indicating an RBD independent approach. To further improve its binding and inhibition efficacy, the S2A2C1 aptamer was conjugated with a reported anti-RBD aptamer, S1B6C3, using suitable linkers to construct fusion aptamers, among which S1B6C3-A5-S2A2C1 has the best binding affinity on WT S1S2, as well as the best inhibition efficacy against the WT S1S2 protein/hACE2. Both S2A2C1 and S1B6C3-A5-S2A2C1 maintain high inhibition efficacy in preventing Delta or Omicron S1S2 protein binding to hACE2, making them well-suited as diagnostic and therapeutic molecular tools against SARS-CoV-2 and its variants. In addition, the past 20 years have witnessed three fatal and well-documented zoonotic coronaviruses (CoVs) outbreaks: SARS-CoV-1 in 2002, MERS-CoV in 2012, and SARS-CoV-2 in late 2019. Ecological reality and current scientific evidence indicate that a new coronavirus may evolve in the forthcoming future. The approach described herein to discover aptamer inhibitors targeting the relatively conserved S2 domain, as well as the strategy to design fusion aptamers, can also be used to target any new coronaviruses as they emerge in the near future.

### Experimental Methods and Materials

### Chemicals and Reagents

All proteins including hACE2, the WT, and variant SARS-CoV-2 S proteins used in this example were purchased from Sino Biological and used without further purification. All aptamers and other nucleic acids were obtained from Integrated DNA Technologies, Inc. as lyophilized powders and were dissolved in nanopure water upon receipt. All chemicals were purchased from Sigma unless mentioned otherwise.

### SELEX Procedure

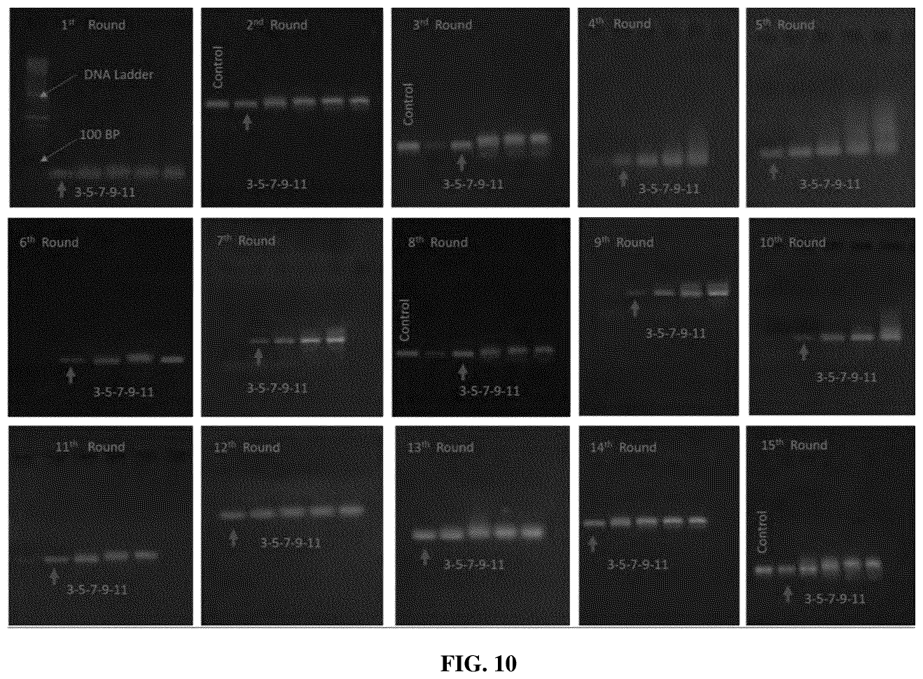

The DNA-SELEX was performed using the S2 domain of the WT spike protein as a target. An oligonucleotide library obtained from IDT which is composed of 40 random nucleotides flanked by constant primer sequences (Table 2) was used. For the first round of the selection, the 100 pmol of the S2 protein and 1 μL of nickel nitrilotriacetic acid (Ni-NTA) beads (G-bioscience) were diluted into 100 μL of SELEX buffer (PBST-Mg buffer, PBS with 1 mM MgCl₂, pH 7.4, 0.01% tween) and incubated at room temperature (RT), rotating for 1 h. Meanwhile, 3 nmol of DNA library was diluted into 100 μL of PBST-Mg and treated at 95° C. for 5 min, on ice (or 4° C.) for 5 min, RT for 5 min, and placed in ice. When 1 h of incubation was completed, protein-bead (P-B) complex was washed two times by 200 μL SELEX buffer and combined with heat-treated DNA library, with 1 μL of 100 times concentrated tRNA, and incubated for 1 h at RT with rotation. After incubation, the protein-bead-library (PBL) complex was washed two times by 200 μL SELEX buffer to remove the unspecific library. After washing, the bound library was eluted 2 times by 30 μL of hot water at 95° C. The selected library was amplified by the polymerase chain reaction (PCR). For the first round of selection, the PCR mixture contains 60 μL of the library, 39 μL of nuclease-free water, 100 μL of 2×PCR solution, and 1 μL of Easy Taq polymerase. The 50 μL of the PCR mixture was loaded into each PCR tube and amplified in the conditions of 2 min at 95° C.; 9 cycles of 45 s at 95° C.; 30 s at 54° C.; 30 s at 72° C., 2 min at 72° C. After completing PCR, all PCR product was collected in a tube. To optimize the PCR cycle number for bulk amplification, 5 μL of the PCR product, 119 μL water, 125 μL of 2×PCR solution, and 1.25 μL Easy Taq polymerase were mixed in a tube and then distributed equally (50 μL) into 5 PCR tubes. Amplification conditions were: 2 min at 95° C.; 3-11 cycles of 45 s at 95° C.; 30 s at 54° C.; 30 s at 72° C., 2 min at 72° C. The PCR tubes were taken out in 3, 5, 7, 9, and 11 cycles, respectively, and kept in ice. Then PCR products were assessed with 2% agarose gel electrophoresis to determine the suitable number of PCR cycles (X). The suitable number of the PCR cycles provides the right PCR product which was confirmed by a brighter and smear-free band at 73 base pairs (FIG. 10). Once the number of suitable PCR cycles (X) was determined, the bulk PCR reaction was run to generate 1 (or 2) mL of PCR mixture (20 μL of the 1^{st} round PCR solution, 475 μL water, 500 μL of 2×PCR solution, and 5 μL of Easy Taq Polymerase). The PCR amplification conditions were set to be 2 min at 95° C.; X cycles of 45 s at 95° C.; 30 s at 54° C.; 30 s at 72° C.; 2 min at 72° C.

TABLE 2
Details of the selection cycles

| | | Counter Selection | Binding | | | Revised |
|---|---|---|---|---|---|---|
| Selection | S2-Protein | (Ni-NTA bead/ | Time | Washing | Total PCR | Yield |
| Round | (pmol) | incubation time) | (min) | repetition | Cycles | (μg) |
| 1 | 100 | | 60 | 2 | 9 + 3 = 12 | 3.9 |
| 2 | 100 | | 60 | 2 | 9 + 5 = 14 | 2.5 |
| 3 | 100 | 2 μL, 5 min | 60 | 3 | 9 + 4 = 13 | 1.7 |
| 4 | 50 | | 50 | 3 | 9 + 4 = 13 | 1.8 |
| 5 | 50 | 2 μL, 10 min | 50 | 4 | 9 + 3 = 12 | 1.9 |
| 6 | 50 | 2 μL, 10 min | 50 | 4 | 7 + 5 = 12 | 2.2 |
| 7 | 50 | | 50 | 5 | 7 + 5 = 12 | 3.0 |
| 8 | 33 | 2.5 μL, 15 min | 40 | 5 | 7 + 5 = 12 | 3.3 |
| 9 | 33 | 2.5 μL, 15 min | 40 | 6 | 7 + 5 = 12 | 3.4 |
| 10 | 33 | | 30 | 6 | 7 + 5 = 12 | 3.5 |
| 11 | 33 | 2.5 μL, 20 min | 30 | 7 | 7 + 5 = 12 | 8.6 |
| 12 | 33 | 3 μL, 20 min | 20 | 7 | 7 + 3 = 10 | 9.3 |
| 13 | 33 | | 20 | 8 | 7 + 3 = 10 | 9.6 |
| 14 | 33 | 4 μL, 25 min | 15 | 8 | 7 + 3 = 10 | 9.4 |
| 15 | 33 | | 15 | 9 | 7 + 3 = 10 | 9.6 |

After a bulk PCR, 20 μL of neutravidin beads were washed two times by 400 μL of SELEX buffer and incubated with 1 mL of PCR products for 15 min rotating at RT. Then the beads were washed two times by 400 μL of the SELEX buffer. The sense strand was separated from the beads by denaturing in 200 μL of 100 mM NaOH solution for 1 min; the solution was immediately neutralized by 0.2 M HCl. Then the beads were again eluted by 212 μL of SELEX buffer, combined with the previous solution, and centrifuged using a desalting column (3K) at 12000 g for 10 min. The remaining solution in the desalting column was washed two times using 400 μL of the SELEX buffer. The eluted library was quantified by nanodrop, then it was treated at 95° C. for 5 min, ice for 5 min, and RT for 5 min and stored at −20° C.

For a subsequent round of selection, the 100 pmol DNA library (˜2 μg) was incubated with 100 pmol protein (bead complex). The amount of protein and incubation time were consistently decreased for following selection rounds to increase the selection pressure (Table 2). The number of washes to the PBL complex was consistently increased to ensure the removal of the unspecific libraries. The bound libraries were eluted two times by 30 μL of hot water at 95° C. Then 20 μL from the total 60 μL elution was used for the PCR amplification, and the remaining 40 μL was stored at −20° C. The PCR amplification, purification, desalting, and quantification were similarly followed as explained before. However, after the second SELEX, the counter selection (CS) was introduced in each alternative round. For that, the ssDNA was incubated with the unembellished Ni-NTA magnetic beads, and the unspecific library bound to the magnetic bead was discarded while the specific library present in the supernatant was used to start the next round of selection. The schematic representation of the DNA SELEX is shown in FIG. 2A.

### Plasmid Preparation for DNA Sequencing

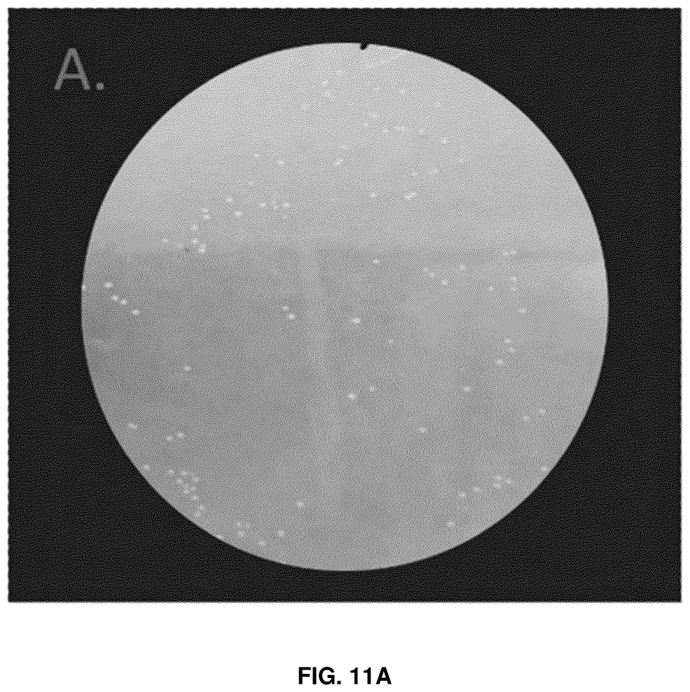

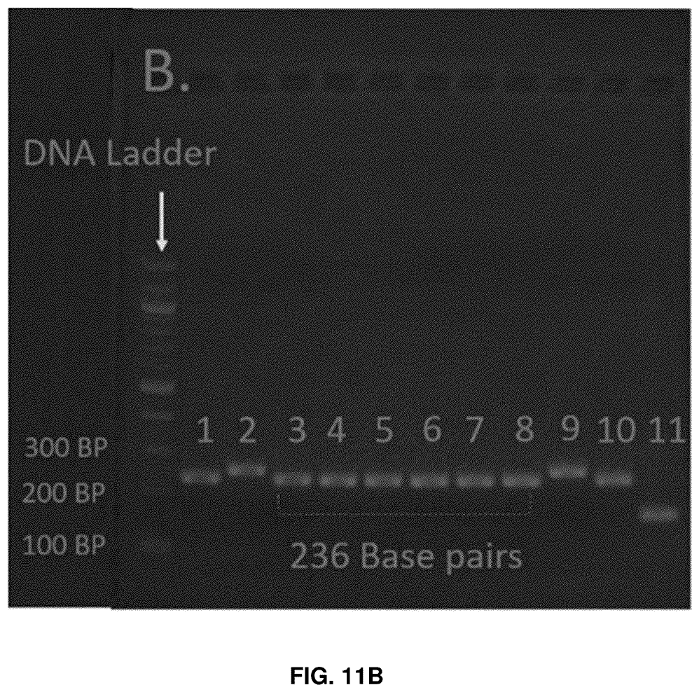

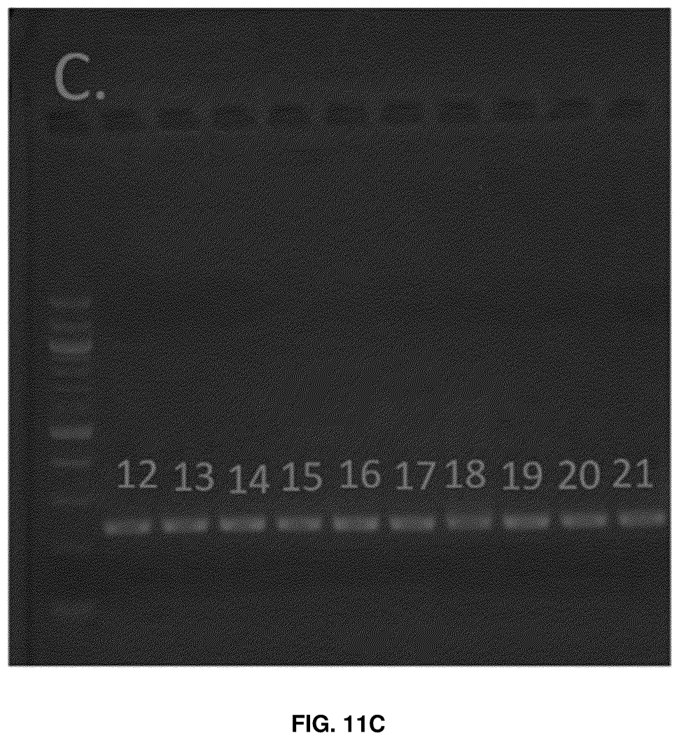

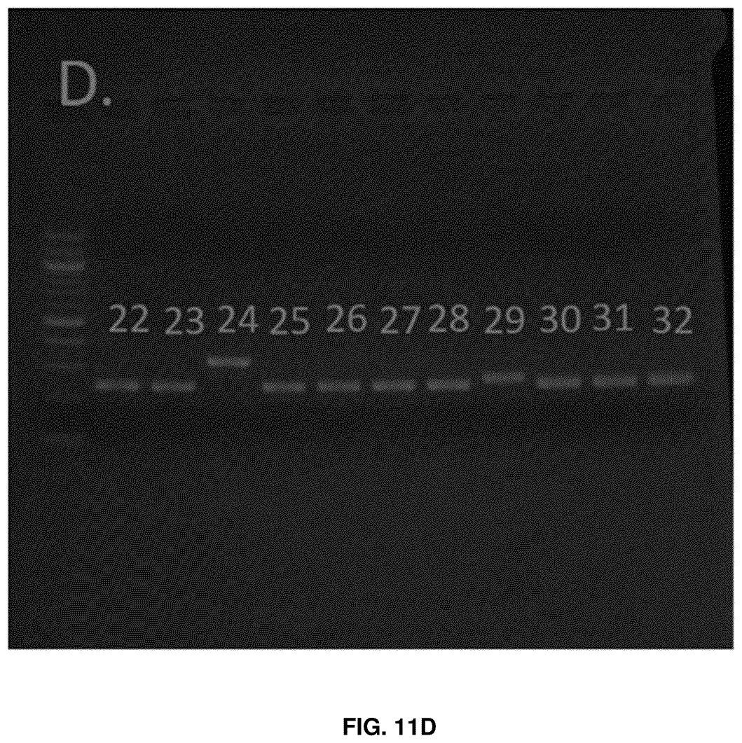

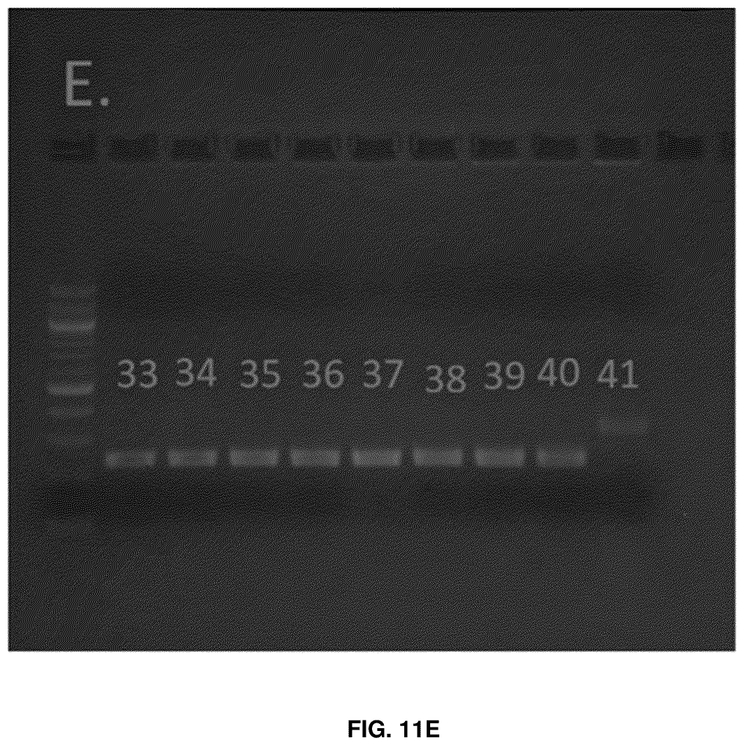

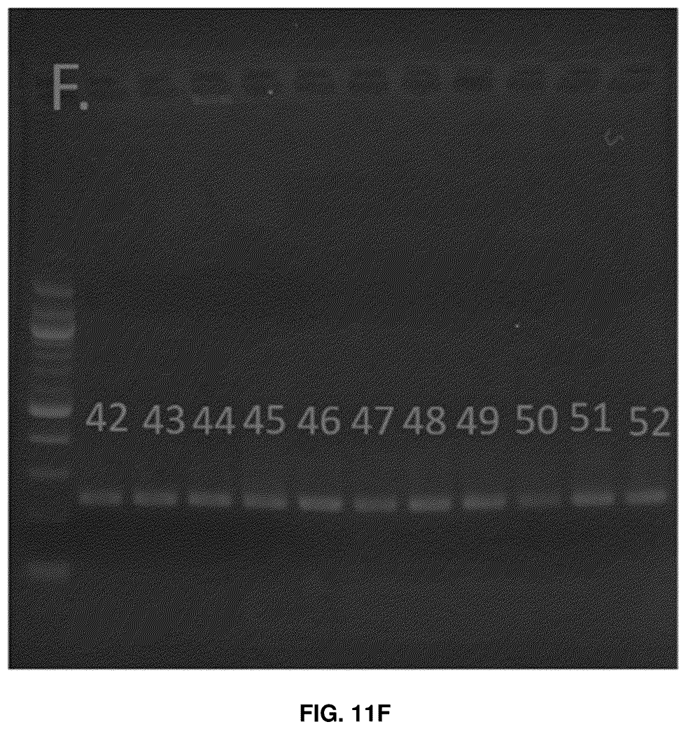

The label-free dsDNA obtained from the 15^{th} selection round was purified using NucleoSpin Gel and PCR Clean-up kit (Ref. #740609-250) and used as an insert. The TOPO TA cloning kit (Ref. #45-0071) was used for ligation, and E. coli component cells were transformed using recombinant DNA. The ampicillin-resistant bacterial colonies were cultured on the ampicillin (100 μg/mL) containing Luria broth (LB) agar plate (follow the standard protocol). The bacterial culture was subjected to PCR to assess the correct insert using gel electrophoresis (FIGS. 11A-11F). The plasmid was extracted from the bacterial solution having desirable insert using E.Z.N.A.® Plasmid DNA Mini Kit (Ref. #D6942-01) and sequenced by the Human Genetics Comprehensive Cancer Center DNA Sequencing Facility at the University of Chicago.

### ELISA Assay With HRP Anti-His Tagged Antibody

The 0.5 μg hACE2 in 50 μL 0.1 M NaHCO₃ (pH 8.6) was added to the high binding 96 well plates (Fisher brand #REF 12565501) and incubated overnight. The solution was removed and incubated with 100 μL of 5 mg/mL BSA in 0.1 M NaHCO₃ for 1 h at RT and washed 3 times with 200 μL SELEX buffer containing tween 20. Then, the 50 μL solution of 100 nM His-tagged S1S2-protein and the aptamer in SELEX buffer was incubated at RT for 1 h. The plate was then washed six times with 200 μL SELEX buffer to remove the unbound S1S2-protein. Then 50 μL of 2000 times diluted anti-His-tagged HRP antibody in SELEX buffer was added, which can bind to remaining His-tagged S1S2-protein. The wells were incubated at RT for 30 min and washed six times. When the aptamer shows the capacity to block S1S2/hACE2 interaction, HRP will not persist in the well plate after washing. Finally, 50 μL of TMB substrate solution was added to the well and incubated for 30 minutes at RT. The intense blue color produced in this step refers that a treatment does not efficiently block S1S2/hACE2 interaction. When 2 μL of concentrated sulfuric acid was added to the blue product, the yellow color was formed. The absorbance of the yellow product was measured at λₘₐₓ=450 nm using the Clariostar microplate reader (BMG LABTECH).

### Fluorescence Microscope-Based Binding Assay

The 20 μL of 10×diluted Hispur Ni-NTA resin bead (#REF. 88221; Thermo scientific) was washed two times with 500 μL of SELEX buffer, resuspended in 50 μL of SELEX buffer, and incubated with 5 pmol of His-tag target protein for 30 minutes at RT with rotation. The resin-protein complex was washed two times with 500 μL of SELEX buffer to remove unbound protein and resuspended with 50 μL of SELEX buffer. The 10 pmol of 6-FAM-labeled ssDNA or aptamer was incubated in this resin-protein complex at RT for 30 min. After washing two times with 500 μL of SELEX buffer, the complex was finally resuspended in 50 μL of SELEX buffer and transferred on a glass slide for the fluorescence measurement. The fluorescence image was collected using both the green fluorescence and transmitted light channels by the digital inverted fluorescence microscope (Invitrogen EVOS FL).

### Determination of Binding Affinity

The 100 nM His-tagged protein and anti-His tagged biotinylated antibody were incubated at RT for 2 h in 50 μL of SELEX buffer. The protein-antibody complex was then incubated with 2 μL of sera-mag magnetic streptavidin-coated particles (Ref. #30152103010150, Cytiva) at RT for 2 h and stabilized overnight at 4° C. After washing two times with 200 μL of SELEX buffer, the protein-bead complex was incubated with the 6-FAM-labeled aptamer of the concentration 3, 10, 30, 100, 300, and 1000 nM, respectively, in 1.5 mL Eppendorf tubes for 2 h. The unbound aptamer was removed by washing three times with 200 μL of SELEX buffer; the bound aptamer was eluted using 30 μL of hot SELEX buffer at 95° C. The fluorescence intensity from the sample at 520 nm was collected using the Clariostar microplate reader (BMG LABTECH), plotted against concentration using Origin Software to calculate the binding affinity (K_{d}).

### Synthesis of Gold Nanoparticles

The three-necked round bottom flask was cleaned with freshly prepared aqua regia (concentrated HNO₃ and HCl in 1:3 molar ratio), rinsed with nuclease-free water, and perfectly dried before use. The AuNPs colloid was synthesized from KAuCl4 (Ref. #334545-1G; Sigma Aldrich) precursor using the classical citrate reduction method. Briefly, a 100 mL of 1 mM KAuCl₄ solution was heated to boiling. Then, 2 mL of 194 mM sodium citrate solution (CAS #1545801, Sigma Aldrich) was added and boiled for an additional 15 min with good stirring. The color of the solution changes from yellow, clear/gray, and finally to dark wine red. After 15 min of boiling the reaction, the flask was taken out and cooled slowly to room temperature.

### Flow Cytometry Experiment to Measure the Binding Efficacy of the Aptamers

The 100 μL of 500 nM His-tagged target protein was prepared in SELEX buffer and incubated with 1 μL of Ni-NTA magnetic beads (G-Biosciences, #REF 062N-A), rotating for 1 h at RT. The bead/protein complex was washed twice by 200 μL of SELEX buffer. Then, 50 pmol of 6-FAM-labeled aptamer was prepared in 100 μL of SELEX buffer, pre-treated at 95° C., ice (or 4° C.), and then RT (each for 5 min), was incubated with the bead/protein for 1 h at RT with rotation. After incubation, the beads were washed two times with 200 μL of SELEX buffer and finally resuspended with 100 μL of SELEX buffer. The 6-FAM-labeled aptamers bound on protein/bead were analyzed by Flow cytometry (Guava easyCyte 5HT, Catalog #0500-4005) counting approximately 5000 events.

### Flow Cytometry Experiment to Measure the Binding Affinity of the S1B6C3-A5-A2A2C1

The 50 μL of 200 nM His-tagged spike-protein was prepared in SELEX buffer and incubated with 1 μL of Ni-NTA 2×diluted magnetic beads (G-Biosciences, Ref. #062N-A), rotating for 1 h at RT. The bead/protein complex was washed twice with 100 μL of SELEX buffer. Then, 1, 3, 10, 30, 100, 300, and 1000 nM solutions of 6-FAM-labeled aptamer were prepared in 50 μL of SELEX buffer, pre-treated at 95° C., ice (or 4° C.), and then RT (each for 5 min), was incubated with the bead/protein for 1 h at RT with rotation. After incubation, the beads were washed two times with 100 μL of SELEX buffer and finally resuspended with 50 μL of SELEX buffer. The 6-FAM-labeled aptamers bound on protein/bead were analyzed by flow cytometry (Guava easyCyte 5HT, Catalog #0500-4005) counting approximately 5000 events. Each experiment was performed for three trials, and integrated flow events to calculate the K_{d}.

### Example II—A Universal DNA Aptamer as an Efficient Inhibitor Against Spike Protein/hACE2 Interactions

A universal aptamer against spike proteins of diverse SARS-CoV-2 variants was discovered via DNA SELEX towards the wild-type (WT) S1S2 protein. This aptamer, A1C1, binds to the S1S2 protein of WT or other variants of concern such as Delta and Omicron with low nanomolar affinities. A1C1 inhibited the interaction between hACE2 and various S1S2 proteins by 85-89%. This universal A1C1 aptamer can be used in diagnostic and therapeutic molecular tools to target SARS-CoV-2 and its variants.

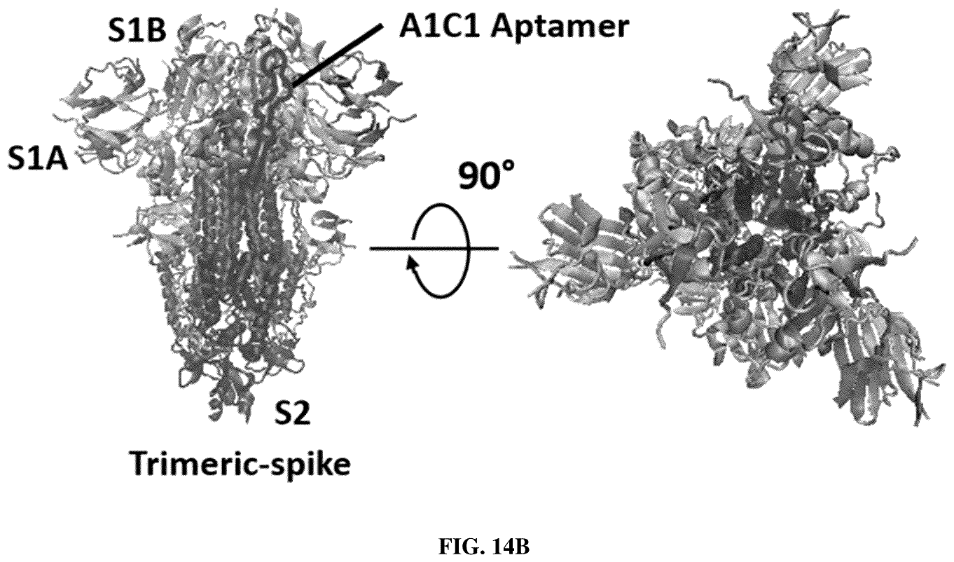

Severe acute respiratory syndrome coronavirus 2 (SARS-CoV-2) uses its spike protein (S protein) to attach to the host cell via human angiotensin converting enzyme 2 (hACE2). The viral infection can be stopped by an inhibitor that can block the interaction between the S protein and hACE2. As shown in FIGS. 14A-14B, one S protein includes three S1S2 proteins, and each S1S2 is composed of the subunits S1 and S2. S1 consists of S1A and S1B (FIG. 14A), in which S1B, also called the receptor-binding domain (RBD), establishes the direct interaction with hACE2. Additionally, the S2 subunit plays a function in mediating the fusion of the viral membrane to the host cell. Therefore, virus entry is accomplished via a cascade of events, i.e., S1 binds to hACE2, which then triggers S2 to change its conformation to a more stable post-fusion state and allows viral entry into the host cell. Since S1 directly interacts with hACE2, biomolecules such as aptamers may effectively block the interaction between S1 and hACE2.

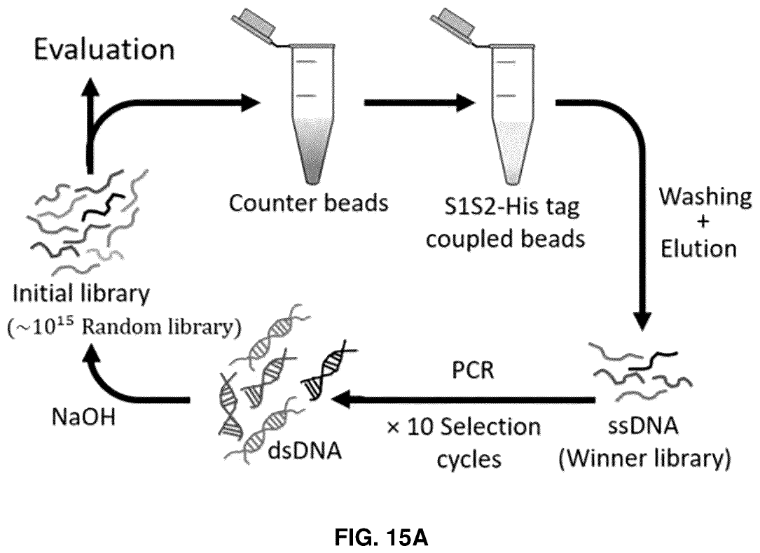

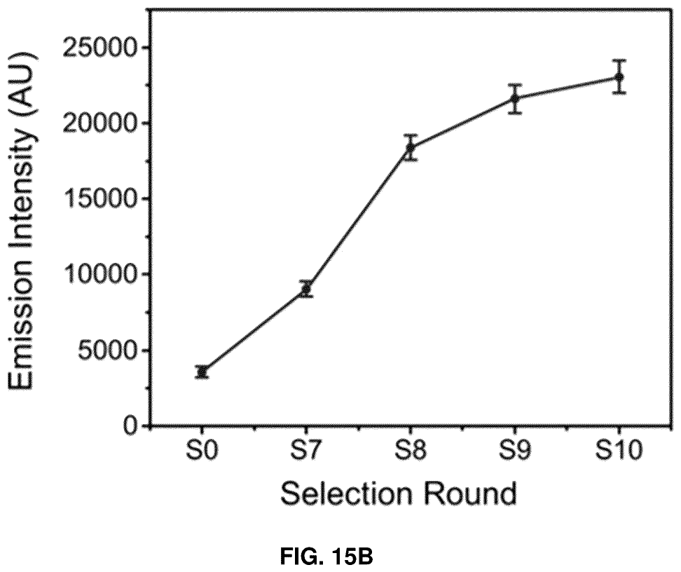

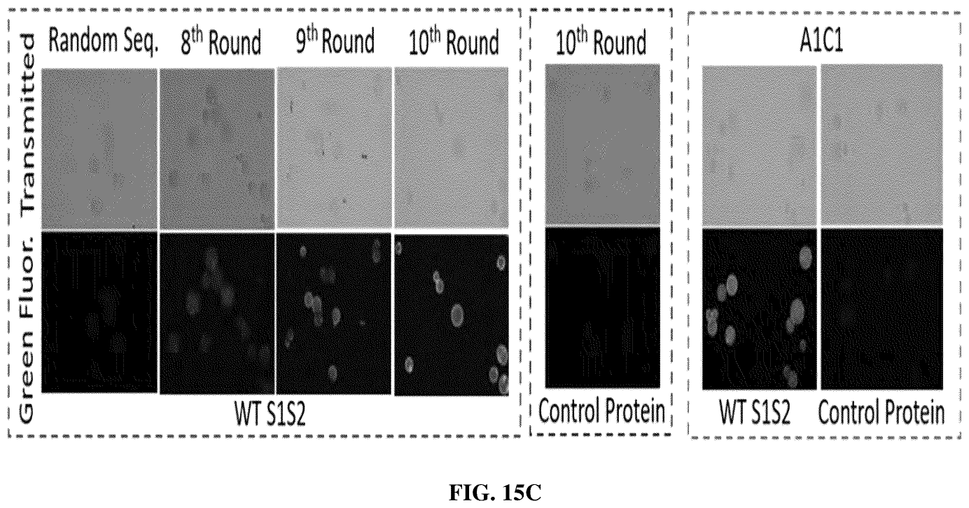

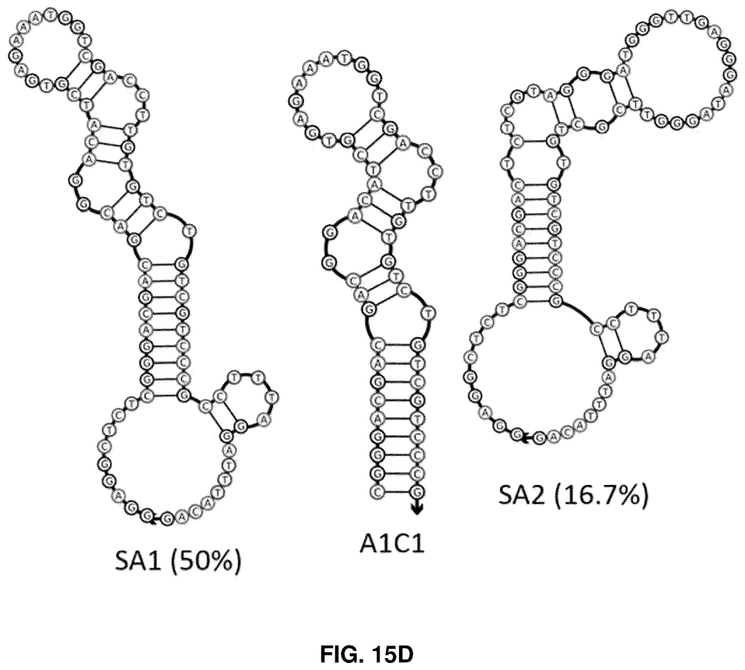

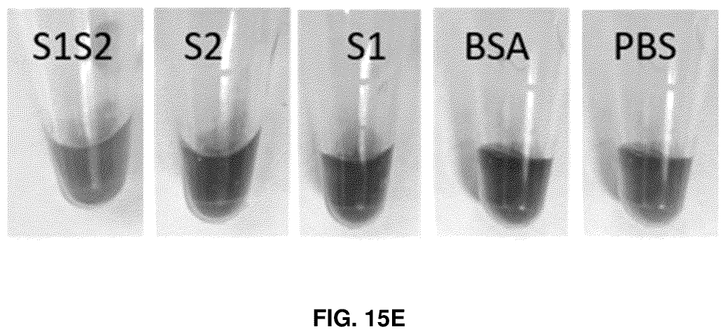

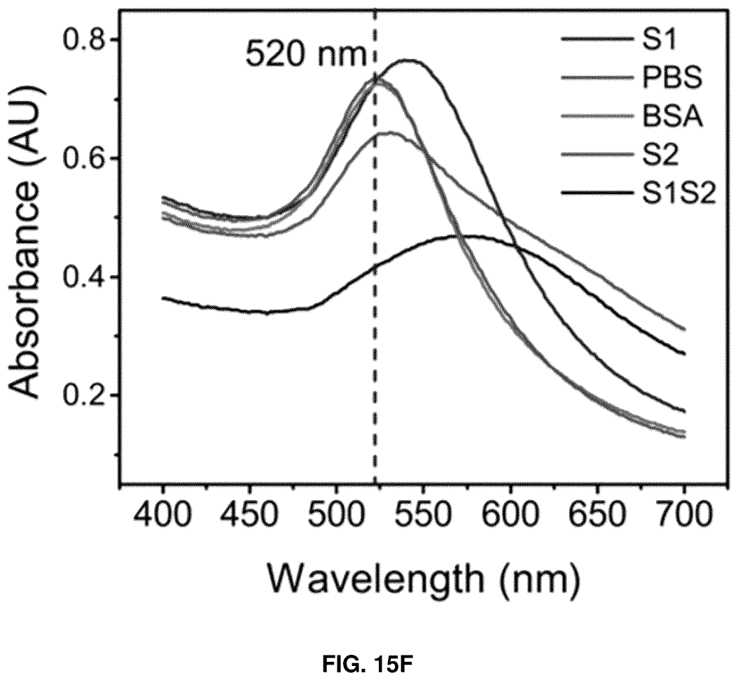

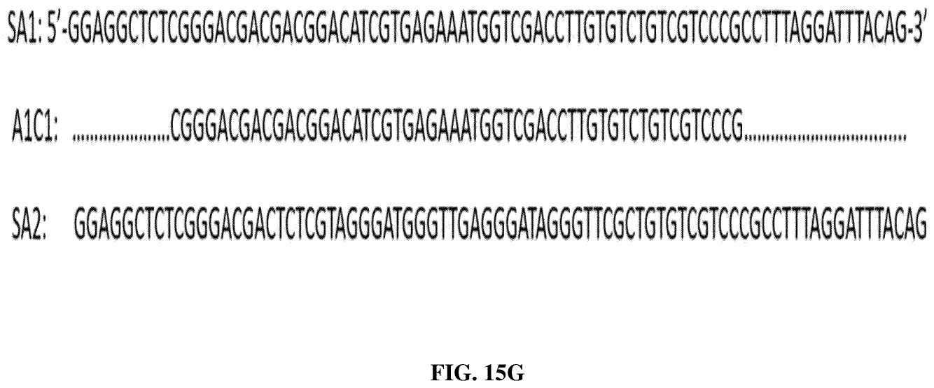

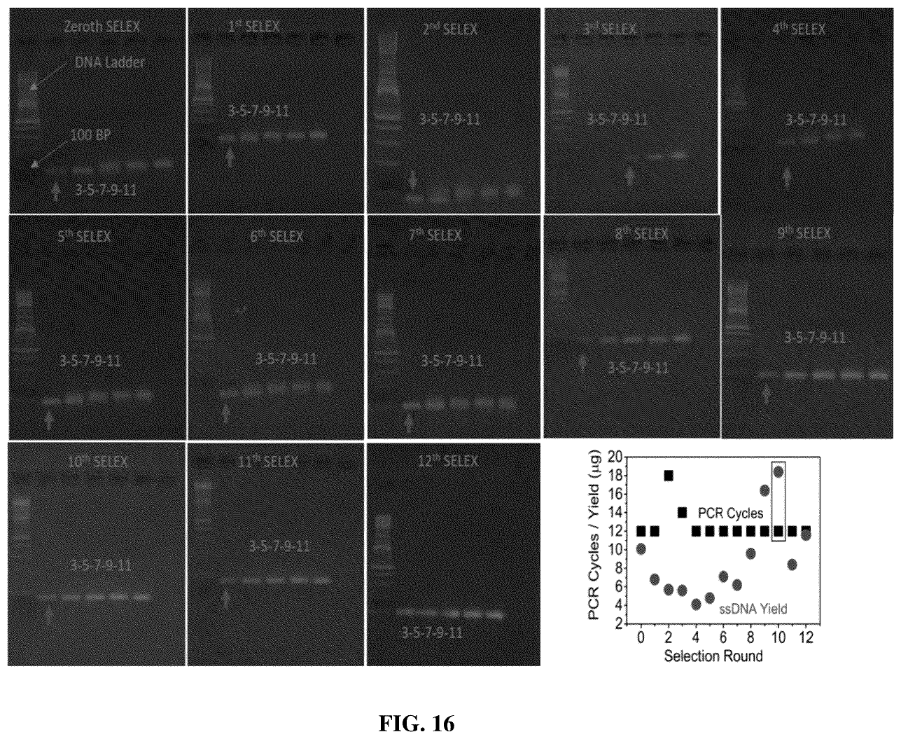

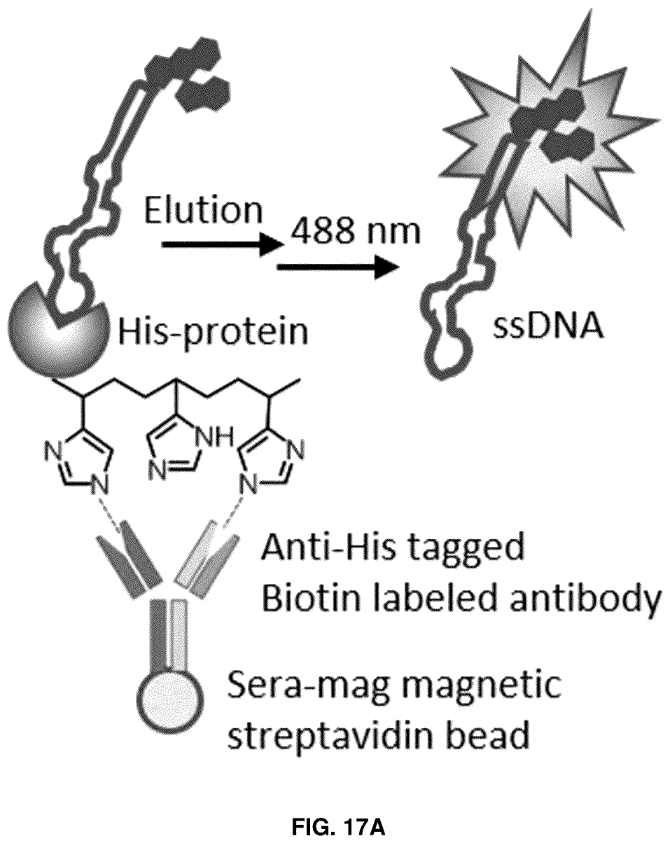

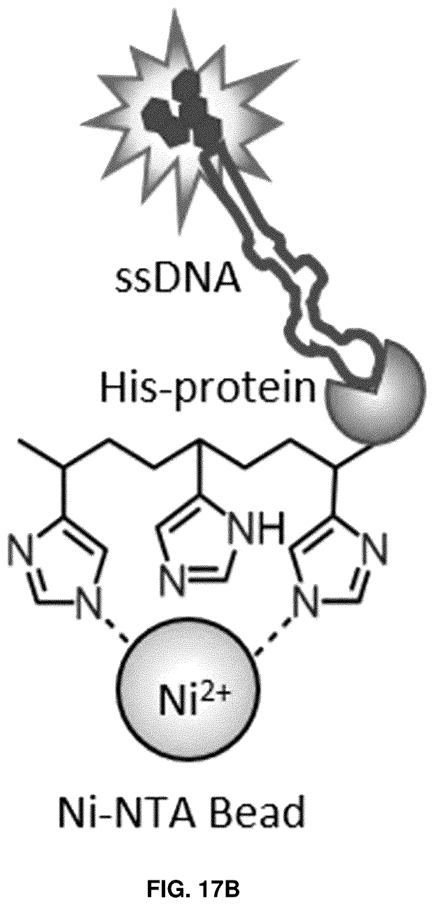

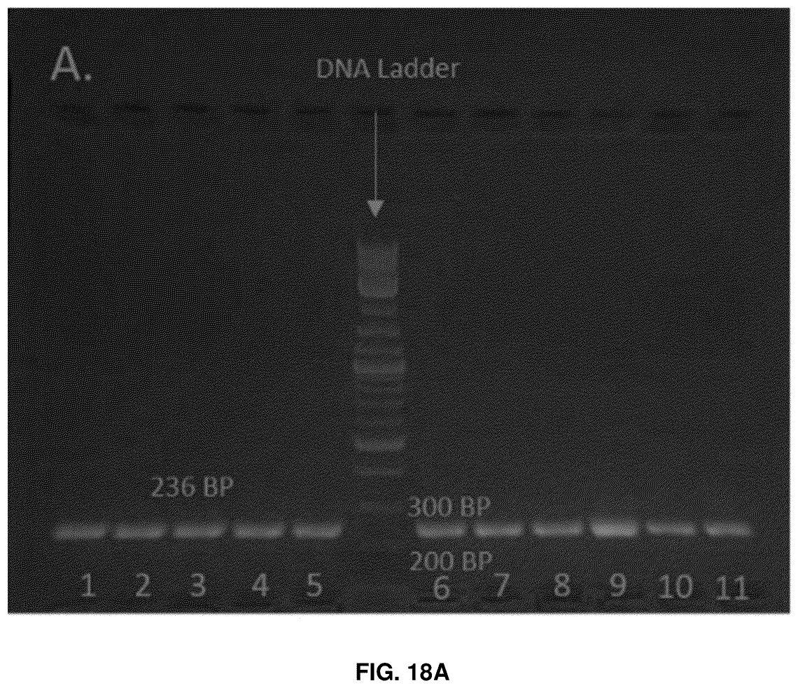

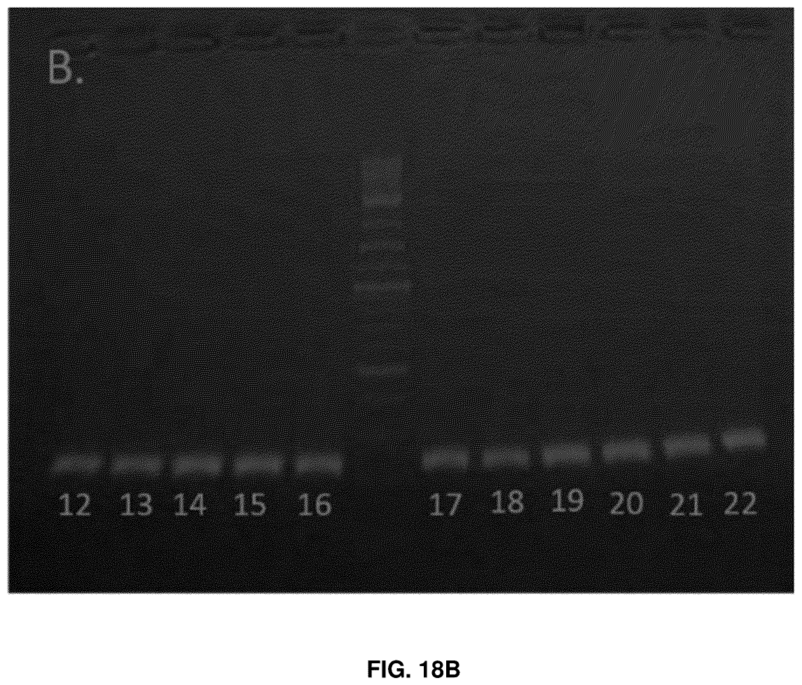

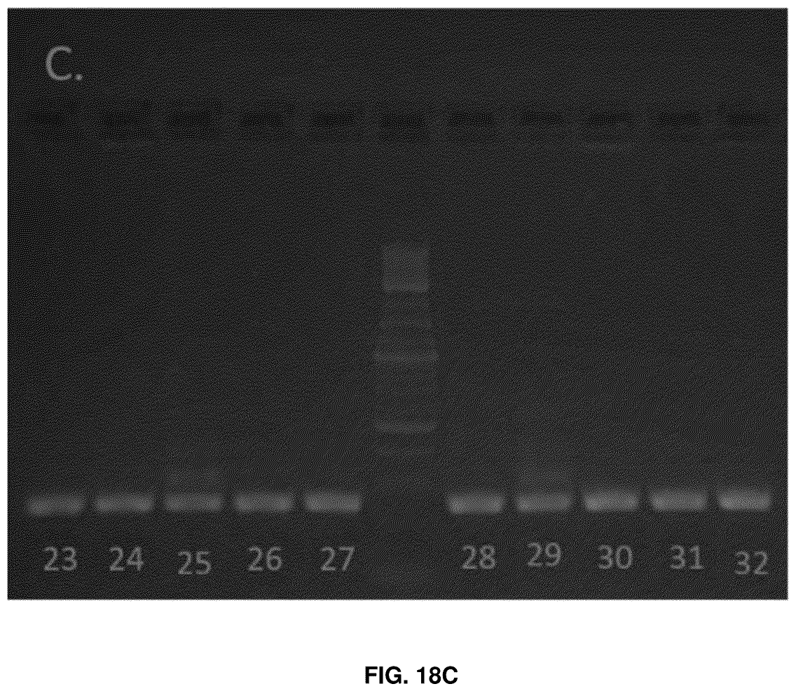

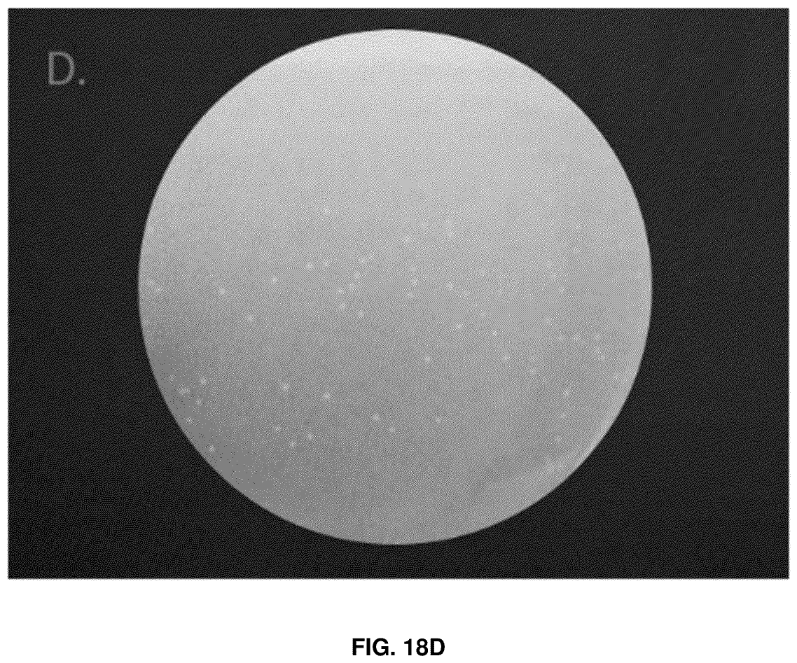

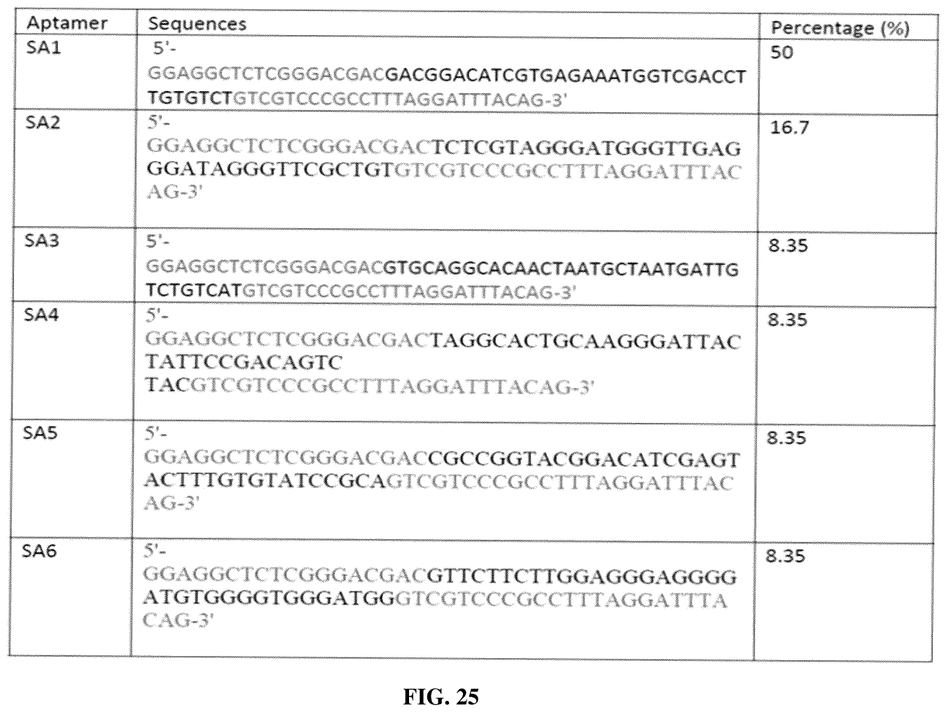

In this example, the WT S1S2 was used as the target, and an in vitro selection was performed to isolate aptamers (FIG. 15A). The initial pool of DNA libraries contained ˜10¹⁵ unique sequences of a 40-nucleotides randomized region. After each round of selection, the winner DNA library was amplified by PCR reaction. After the second selection round, the counter-selection was employed to remove the nonspecific library in each alternative selection round. The details of the selection rounds are provided in FIG. 16 and Table 4. Moreover, the target-to-library ratio was incrementally decreased from 2.4:1 in round 1 to 1:5 in round 10 to favor the selection of high-affinity anti-S1S2 aptamers. After 10 rounds of in vitro selection, the enriched library was obtained (FIG. 16). The binding capabilities of all enriched DNA toward S1S2 were evaluated by a fluorescence plate reader after the 7^{th} selection round. Briefly, the 6-FAM-ssDNA was subjected to bind to the His-tagged WT S1S2 protein coupled with anti-His-tagged biotinylated sera-mag magnetic streptavidin-coated beads (FIG. 17A). After washing, the 6-FAM-ssDNA library was eluted from the complex, and fluorescence emission (2520 mm) was measured using the Clariostar microplate reader. As shown in FIG. 15B, the fluorescence signal significantly increased from the 7^{th} round to the 10^{th} round, and it consistently increased to its maximum at the 10^{th} round. In addition, the His-tagged S1S2 protein was immobilized on a nickel-nitrilotriacetic acid (Ni-NTA) resin and incubated with 6-FAM-labeled ssDNAs (FIG. 17B). After washing, the resin was subjected to fluorescence imaging. As shown in FIG. 15C and Table 5, the pool from the 9^{th} and 10^{th} rounds displayed a stronger fluorescent signal than that from the 8^{th} round. Furthermore, the 6-FAM-labeled ssDNA from the 10^{th} round did not bind to the control His-tagged protein immobilized on the Ni-NTA resin. This indicates that there is high binding capability and specificity of the ssDNA library from the 10^{th} round toward the S1S2 protein. The above data indicate that after 10 rounds of SELEX, a useful aptamer candidate pool with good binding ability against the S1S2 protein was obtained. For this reason, the enriched aptamer candidate pool from the 10^{th} round was cloned using a TOPO TA Cloning® Kit. The product of recombination was used to transform E. coli component cells, and random colonies were sequenced (FIGS. 18A-18D). The 50% and 16.7% of the total sequence data were occupied by SA1 and SA2 sequences, respectively (FIG. 25). Both consensus sequence motifs contained multiple GGG, GG, CCC, or CC repeats. The predominant sequence SA1 was optimized by deletion of redundant nucleotides to obtain the aptamer A1C1. The sequence information and secondary structures of all aptamers are shown in FIGS. 15D, 15G. In addition, the specific binding of the 6-FAM-labeled A1C1 on S1S2 was also determined using fluorescence microscopy (FIG. 15C). The Ni-NTA/WT S1S2 complex was incubated with 100 nM 6-FAM-A1C1 DNA for 30 minutes and washed 3 times before collecting fluorescence images by a digital inverted fluorescence microscope (Invitrogen EVOS FL). The bright fluorescence image was observed from the Ni-NTA/WT S1S2 complex in the presence of the 6-PAM-A1C1 aptamer or the 6-FAM-seDNA obtained from the 10^{th} round of selection. However, both of them failed to provide the noticeable bright image when WT S1S2 was replaced by the control protein while keeping all other experimental conditions the same. These experimental results indicate that A1C1 is specifically bound against WT S1S2 with high affinity.

TABLE 4
Details of the selection

| | WT S1S2 | Counter Selection | Binding | | | |
|---|---|---|---|---|---|---|
| Selection | Protein | (Ni-NTA bead/ | Time | Washing | Total PCR | Yield |
| Round | (pmol) | incubation time) | (min) | repetition | Cycles | (μg) |
| 0 | 100 | | | | 9 + 3 = 12 | 10.1 |
| 1 | 100 | | 60 | 2 | 9 + 3 = 12 | 6.8 |
| 2 | 100 | | 60 | 2 | 9 + 3 = 12 | 5.7 |
| 3 | 100 | 2 μL, 5 min | 60 | 3 | 9 + 9 = 18 | 5.6 |
| 4 | 50 | | 50 | 3 | 9 + 5 = 14 | 4.1 |
| 5 | 50 | 2 μL, 10 min | 50 | 4 | 9 + 3 = 12 | 4.8 |
| 6 | 50 | 2 μL, 10 min | 50 | 4 | 9 + 3 = 12 | 7.1 |
| 7 | 50 | | 50 | 5 | 9 + 3 = 12 | 6.2 |
| 8 | 25 | 2.5 μL, 15 min | 40 | 5 | 9 + 3 = 12 | 9.6 |
| 9 | 25 | 2.5 μL, 15 min | 40 | 6 | 9 + 3 = 12 | 16.4 |
| 10 | 25 | | 30 | 6 | 9 + 3 = 12 | 18.4 |
| 11 | 25 | 2.5 μL, 20 min | 30 | 7 | 9 + 3 = 12 | 8.4 |
| 12 | 25 | 3 μL, 20 min | 20 | 7 | 9 + 3 = 12 | 11.6 |

TABLE 5
The fluorescence emission from the bound 6-FAM-ssDNA over various selection rounds

| | Emission at λₘₐₓ = 520 nm | | | | | | | |
|---|---|---|---|---|---|---|---|---|
| | | | | After Background Correction | | | | |
| Selection | Before Background Correction | | | | | | | Standard |
| Round | Trial 1 | Trial 2 | Trial 3 | Trial 1 | Trial 2 | Trial 3 | Average | Error |
| Buffer | 558 | 590 | 551 | 0 | 0 | 0 | 0 | 0 |
| 0 | 3570 | 3837 | 3329 | 3570 | 3937 | 3229 | 3578.667 | 354.0796 |
| 7 | 8725 | 9594 | 8822 | 8725 | 9594 | 8822 | 9047 | 476.1922 |
| 8 | 17680 | 19006 | 17476 | 18680 | 19006 | 17476 | 18387.33 | 805.8941 |
| 9 | 20818 | 22379 | 20578 | 21818 | 22379 | 20578 | 21591.67 | 921.5858 |
| 10 | 22580 | 24273 | 22320 | 22580 | 24273 | 22320 | 23057.67 | 1060.508 |

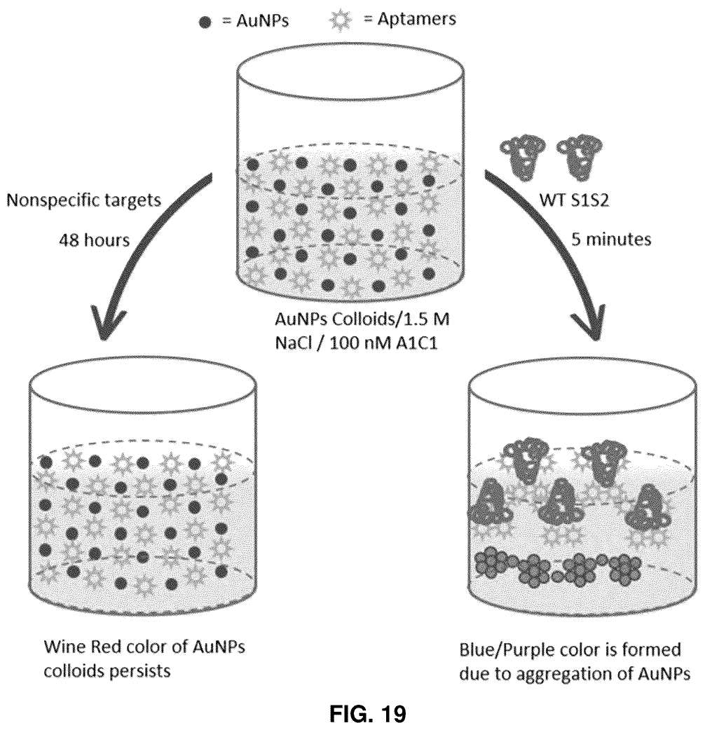

Next, the binding capability of the A1C1 aptamer toward the WT S1S2 was further evaluated by the gold nanoparticle (AuNPs)-based colorimetric assay (FIG. 19). The detailed mechanism and kinetics of the interaction between aptamer and AuNPs that prevents AuNPs from aggregating have been extensively characterized by others. In this example, briefly, when the WT S1S2 protein was added, A1C1 was preferably bound to WT S1S2, and the aggregation was inevitable. The wine-red color of the AuNPs colloids was intact for up to 48 hours in the presence of 1.5 M NaCl and 250 nM A1C1 aptamer. However, the red color dramatically changed into blue or purple within 5 minutes of adding 250 nM of the WT S1S2 protein (FIG. 15E). Besides visual color change, UV-Vis measurement was also employed to measure the effect of various proteins on AuNPs/A1C1/NaCl colloids (FIG. 15F). The WT S1S2 protein caused the significant redshift of the characteristic peak of AuNPs colloids located at 520 nm. The WT S1 and WT S2 proteins also caused some redshift to the characteristic 520 nM peak, but it was not as obvious as the redshift caused by the WT S1S2 protein. This experimental evidence states that A1C1 preferably binds to the intact WT S1S2 protein over the single WT S1 or S2 protein. However, since S2 shows more purple color change than S1, A1C1 binds to the junction site of S1 and S2 in the whole S1S2 protein, and S2 contributes more to the interaction with A1C1 than S1. According to CDC reports, the Delta and Omicron variants have a few mutations in the S1S2 protein, and most mutations are located on the S1 protein, indicating that S2 is highly conserved. Since A1C1 preferably binds to S2 over S1, A1C1 is able to recognize the S1S2 proteins of various SARS-CoV-2 variants, allowing universal recognition of S1S2.

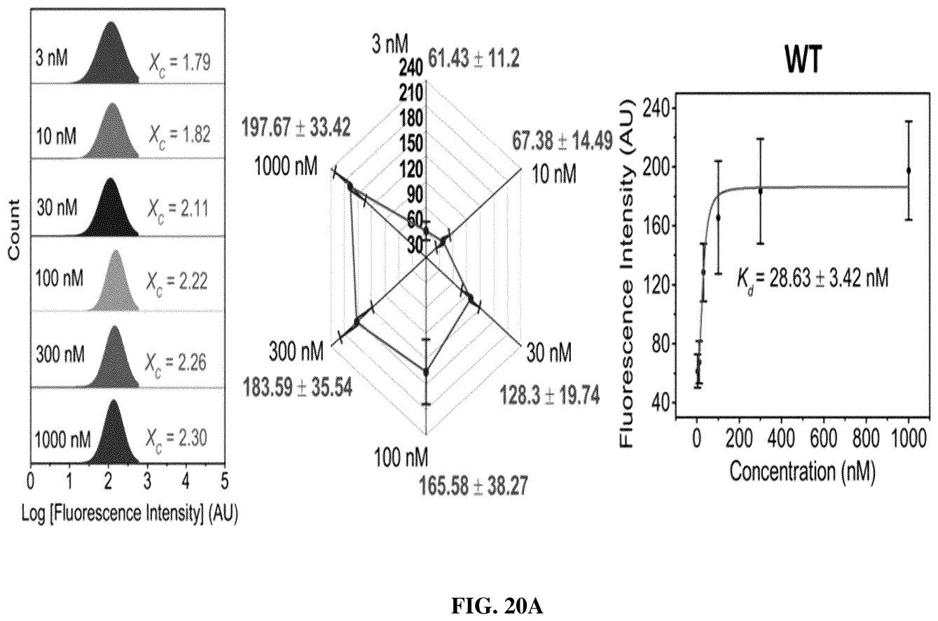

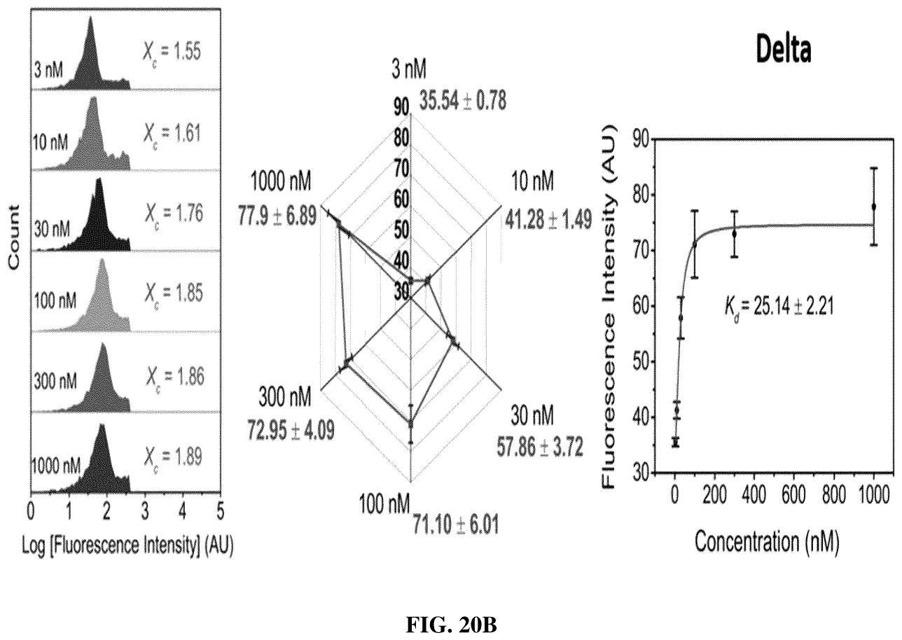

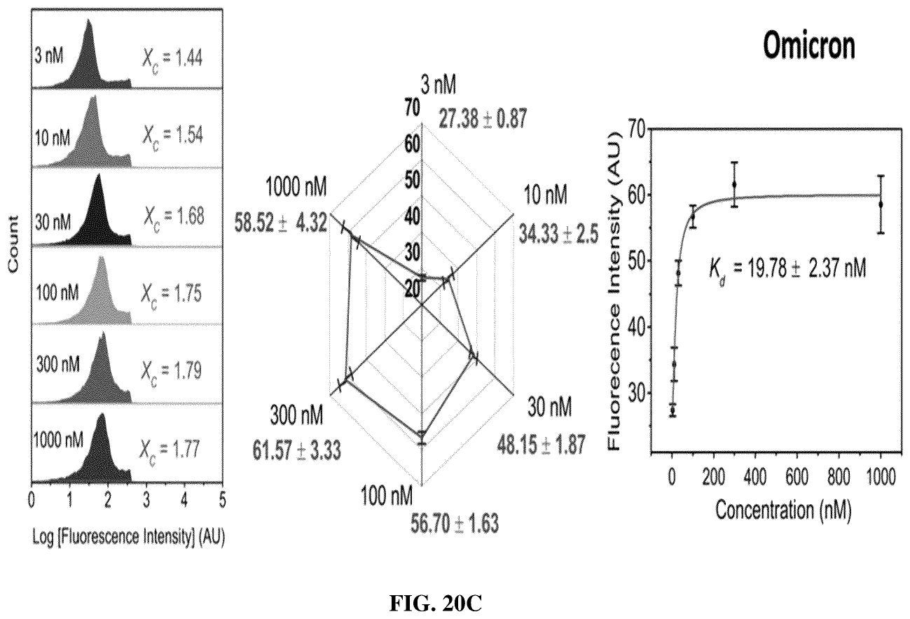

To test the binding affinity of the A1C1 aptamer against WT, Delta, and Omicron S1S2 proteins, the flow cytometry assay using the Ni-NTA magnetic beads was employed (FIGS. 20A-20C). The beads were incubated with 200 nM target protein for 2 h, and it was washed with the SELEX buffer. The Ni-NTA/protein complex was then incubated with various concentrations (3, 10, 30, 100, 300, and 1000 nM) of 6-FAM-A1C1 aptamer, and samples were subjected to the flow cytometry measurement after washing. When the A1C1 concentration was 3 nM, the Ni-NTA/protein bead complex produced a weaker fluorescence signal. When A1C1 concentration was increased from 3 to 10, 30, or 100 nM, the fluorescence signal was significantly increased. However, it did not apparently change when A1C1 concentration was increased from 100 to 300 or 1000 nM. As shown in FIGS. 20A-20C, A1C1 has a low nanomolar binding affinity toward different S1S2 proteins. The K_{d} values are 28.6, 25.1, and 19.8 nM, for the WT, Delta, and Omicron S1S2 proteins, respectively. These data support the previous conclusion that A1C1 binds to a highly conserved region of S1S2.

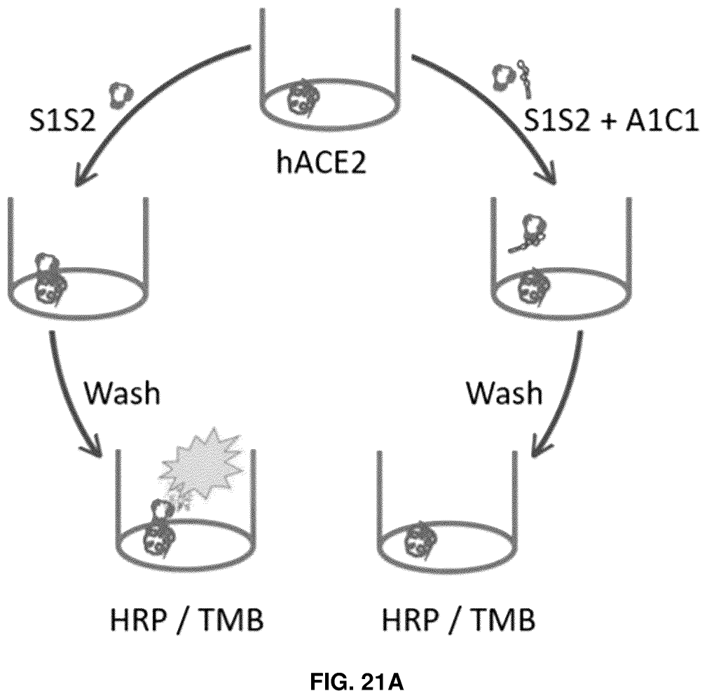

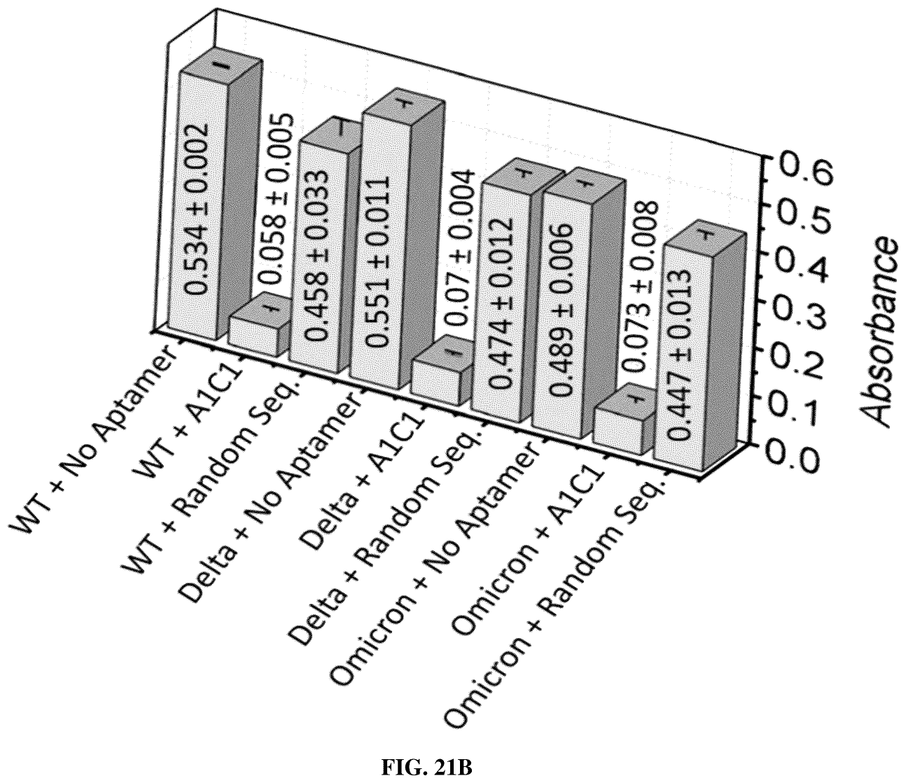

Finally, the A1C1 aptamer was tested for its ability to block the hACE2/S1S2 interaction in an ELISA competition assay (FIG. 21A). Briefly, the ELISA wells were first coated with the hACE2 protein. The A1C1 aptamer and S1S2 proteins (WT, Delta, or Omicron) were then added simultaneously to the well to measure the competition of the hACE2/S1S2 binding over the A1C1/S1S2 interaction. After washing, the amount of the remaining bound S1S2 protein in each well was determined using the absorbance caused by the HRP-mediated oxidation of the TMB, since anti-His-tagged-HRP relies on the presence of His-tagged-S1S2. Additional control experiments were performed in the absence of the A1C1 aptamer or using a control pool of the ssDNA library obtained from the 1^{St} round selection. The analysis from the ELISA competition assay showed that approximately 89.1% of WT, 87.3% of Delta, and 85% of Omicron S1S2 interaction with hACE2 was inhibited by the A1C1 aptamer, respectively (Table 7, and FIG. 21B). The control experiments confirm that the hACE2/S1S2 interaction can only be specifically inhibited by A1C1.

TABLE 7
The measurement of the absorbance (λ₄₅₀ nm) in ELISA competition Assay
to test the inhibition efficacy of A1C1 aptamer against S1S2/hACE2 interaction

| | | Absorbance (λ₄₅₀ nm) | | | | | | | |
|---|---|---|---|---|---|---|---|---|---|
| | | | | | After Background correction | | | | |
| Samples | | Before Background correction | | | | | | | Standard |
| Protein | Aptamer | Trial 1 | Trial 2 | Trial 3 | Trial 1 | Trial 2 | Trial 3 | Average | Error |
| Omicron | Random | 0.472 | 0.495 | 0.495 | 0.432 | 0.455 | 0.455 | 0.447333 | 0.013279 |
| S1S2 | Sequence | | | | | | | | |
| | A1C1 | 0.105 | 0.112 | 0.121 | 0.065 | 0.072 | 0.081 | 0.072667 | 0.008021 |
| | Aptamer | | | | | | | | |
| | W/o | 0.522 | 0.53 | 0.534 | 0.482 | 0.49 | 0.494 | 0.488667 | 0.00611 |
| | Aptamer | | | | | | | | |
| Delta | Random | 0.508 | 0.527 | 0.506 | 0.468 | 0.487 | 0.466 | 0.473667 | 0.01159 |
| S1S2 | Sequence | | | | | | | | |
| | A1C1 | 0.106 | 0.113 | 0.112 | 0.066 | 0.073 | 0.072 | 0.070333 | 0.003786 |
| | Aptamer | | | | | | | | |
| | W/o | 0.578 | 0.598 | 0.597 | 0.538 | 0.558 | 0.557 | 0.551 | 0.011269 |
| | Aptamer | | | | | | | | |
| WT S1S2 | Random | 0.51 | 0.523 | 0.461 | 0.47 | 0.483 | 0.421 | 0.458 | 0.032696 |
| | Sequence | | | | | | | | |
| | A1C1 | 0.098 | 0.103 | 0.093 | 0.058 | 0.063 | 0.053 | 0.058 | 0.005 |
| | Aptamer | | | | | | | | |
| | W/o | 0.573 | 0.572 | 0.576 | 0.533 | 0.532 | 0.536 | 0.533667 | 0.002082 |
| | Aptamer | | | | | | | | |
| NA | TMB | 0.04 | 0.04 | 0.04 | 0 | 0 | 0 | 0 | 0 |

In summary, anti-S1S2 aptamers were isolated by in vitro selection. The selected universal anti-spike protein aptamer, A1C1, binds to WT, delta, and omicron S1S2 with uniformly high affinity. Furthermore, the A1C1 aptamer can inhibit 85-89.1% of the hACE2/S1S2 interaction including WT, Delta, and Omicron variants. Conclusively, the anti-spike universal A1C1 aptamer can be employed in diagnostic and therapeutic molecular tools to target SARS-CoV-2 and its variants.

### Materials and Methods

### Chemicals and Reagents

All proteins used in this example, including hACE2 and the WT and variant SARS-CoV-2 spike proteins, were purchased from Sino Biological and used without further purification. All aptamers and other nucleic acids were obtained from Integrated DNA Technologies, Inc. as lyophilized powders and were dissolved in nanopure water upon receipt. All chemicals were purchased from Sigma unless mentioned otherwise.

### SELEX Procedure

The DNA-SELEX was performed using the S1S2 domain of the WT spike protein as a target. An oligonucleotide library obtained from IDT which was composed of 40 random nucleotides flanked by constant primer sequences was used (Table 5). For the first round of the selection, 100 pmol of the S1S2 protein and 1 μL of nickel nitrilotriacetic acid (Ni-NTA) beads (Ref. #062N-A; G-bioscience) were diluted into 100 μL of SELEX buffer (PBST-Mg buffer, PBS with 1 mM MgCl₂, pH 7.4, 0.01% tween) and incubated at room temperature (RT), rotating for 1 h. Meanwhile, 3 nmol of DNA library was diluted into 100 μL of PBST-Mg and treated at 95° C. for 5 min, on ice (or 4° C.) for 5 min, RT for 5 min, and placed in ice. When 1 h of incubation was completed, the protein-bead (P-B) complex was washed two times by 200 μL SELEX buffer and combined with a heat-treated DNA library, with 1 μL of 100 times concentrated tRNA, and incubated for 1 h at RT with rotation. After incubation, the protein-bead-library (PBL) complex was washed two times with 200 μL SELEX buffer to remove the unspecific library. After washing, the bound library was eluted 2 times by 30 μL of hot water at 95° C. The selected library was amplified by polymerase chain reaction (PCR). For the first round of selection, the PCR mixture contained 60 μL of the library, 39 μL of nuclease-free water, 100 μL of 2×PCR solution, and 1 μL of Easy Taq polymerase. The 50 μL of the PCR mixture was loaded into each PCR tube and amplified in the conditions of 2 min at 95° C.; 9 cycles of 45 s at 95° C.; 30 s at 54° C.; 30 s at 72° C., 2 min at 72° C. After completing PCR, all PCR product was collected in a tube. To optimize the PCR cycle number for bulk amplification, 5 μL of the PCR product, 119 μL water, 125 μL of 2×PCR solution, and 1.25 μL Easy Taq polymerase were mixed in a tube and then distributed equally (50 μL) into 5 PCR tubes. Amplification conditions were 2 min at 95° C.; 3-11 cycles of 45 s at 95° C.; 30 s at 54° C.; 30 s at 72° C., 2 min at 72° C. The PCR tubes were taken out in 3, 5, 7, 9, and 11 cycles, respectively, and kept in ice. Then, PCR products were assessed with 2% agarose gel electrophoresis to determine the suitable number of PCR cycles (X). The suitable number of PCR cycles would provide the right PCR product and was confirmed by a brighter and smear-free band at 73 base pairs (FIG. 16). Once the number of suitable PCR cycles (X) was determined, the bulk PCR reaction was run to generate 1 (or 2) mL of PCR mixture (20 μL of the 1st round PCR solution, 475 μL water, 500 μL of 2×PCR solution, and 5 μL of Easy Taq Polymerase). The PCR amplification conditions were set to 2 min at 95° C.; X cycles of 45 s at 95° C.; 30 s at 54° C.; 30 s at 72° C.; 2 min at 72° C.

After a bulk PCR, 20 L of neutravidin beads were washed two times by 400 μL of SELEX buffer and incubated with 1 mL of PCR products for 15 min rotating at RT. Then, the beads were washed two times with 400 μL of the SELEX buffer. The sense strand was separated from the beads by denaturing in 200 μL of 100 mM NaOH solution for 1 min; the solution was immediately neutralized by 0.2 M HCl. Then, the beads were again eluted by 212 μL of SELEX buffer, combined with the previous solution, and centrifuged using a desalting column (3K) at 12000 g for 10 min. The remaining solution in the desalting column was washed two times using 400 μL of the SELEX buffer. The eluted library was quantified by nanodrop. Then, it was treated at 95° C. for 5 min, ice for 5 min, and RT for 5 min and stored at −20° C.

For subsequent rounds of selection, the 100 pmol DNA library (˜2 μg) was incubated with 100 pmol protein (bead complex). The amount of protein and incubation time were consistently decreased for the following selection rounds to increase the selection pressure (Table 3), while the number of washes to the PBL complex was consistently increased to ensure the removal of the unspecific libraries. The bound libraries were eluted two times by 30 μL of hot water at 95° C. Then, 20 μL from the total 60 μL elution was used for the PCR amplification, and the remaining 40 μL was stored at −20° C. The PCR amplification, purification, desalting, and quantification were similarly followed for the subsequent rounds as they were in the first SELEX. However, after the second SELEX, the counter selection (CS) was introduced in every other round. For that, the ssDNA was incubated with the unembellished Ni-NTA magnetic beads, and the unspecific library bound to the magnetic beads was discarded, while the specific library present in the supernatant was used to start the next round of selection.

### Plasmid Preparation for DNA Sequencing

The label-free dsDNA obtained from the 10^{th} selection round was purified using NucleoSpin Gel and PCR Clean-up kit (Ref #740609-250; Macherey-Nagel) and used as an insert. The TOPO TA cloning kit (Ref. #45-0071; invitrogen) was used for ligation and transformed the E. coli component cells using recombinant DNA. The ampicillin-resistant bacterial colonies were cultured on Luria broth (LB) agar plates containing ampicillin (100 μg/mL), following the standard protocol. The bacterial culture was subjected to PCR to assess the correct insert using gel electrophoresis (FIGS. 18A-18C). The plasmid containing the desirable insert was extracted from the bacterial solution using E.Z.N.A.® Plasmid DNA Mini Kit (Ref. #D6942-01; Omega Bio-Tek) and sequenced by the Human Genetics Comprehensive Cancer Center DNA Sequencing Facility at the University of Chicago.

### ELISA Assay With HRP Anti-His Tag Antibody

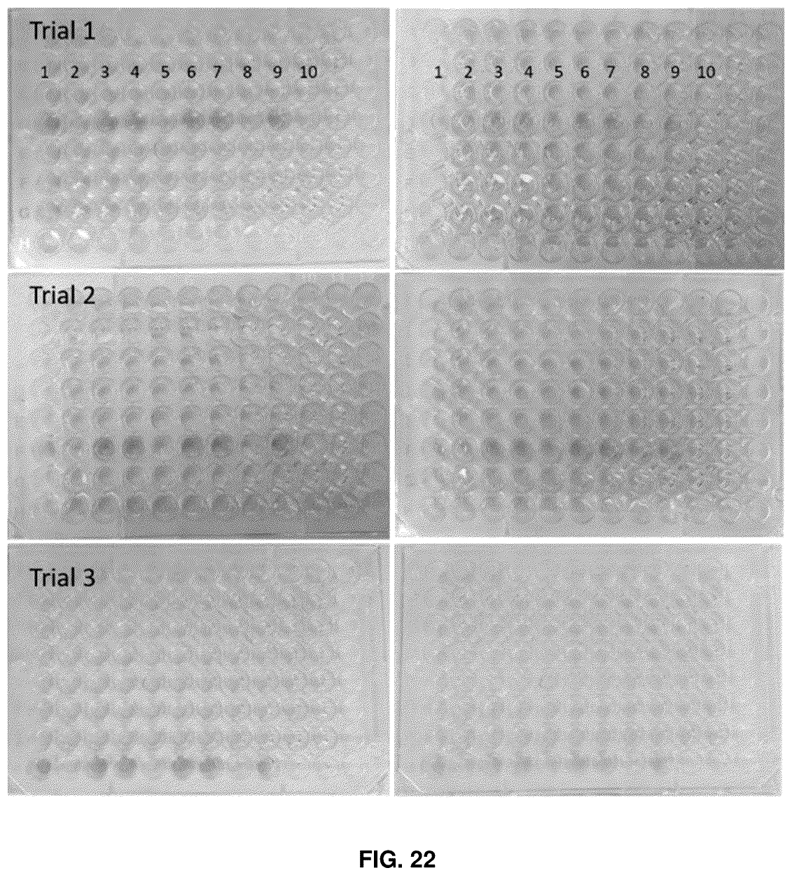

The 0.5 μg hACE2 in 50 μL 0.1 M NaHCO₃ (pH 8.6) was added to the high-binding 96 well plates (Ref #12565501; Fisher brand) and incubated overnight. The solution was removed and incubated with 100 μL of 5 mg/mL BSA in 0.1 M NaHCO₃ for 1 h at RT and washed 3 times with 200 μL SELEX buffer containing Tween 20. Then, a 50 μL solution of 100 nM His-tagged S1S2-protein and the aptamer in SELEX buffer was incubated at RT for 1 h. The plate was then washed six times with 200 μL SELEX buffer to remove the unbound S1S2-protein. Then, 50 μL of 2000 times diluted anti-His-tagged HRP antibody (catalog #AE028; ABclonal) in SELEX buffer was added, which bound to the remaining His-tagged S1S2-protein. The wells were incubated at RT for 30 min and washed six times. When the aptamer shows the capacity to block S1S2/hACE2 interaction, HRP will not persist in the well plate after washing. Finally, 50 μL of TMB substrate solution was added to the well and incubated for 30 minutes at RT. The intense blue color produced in this step was caused by the strong S1S2/hACE2 interaction. When 2 μL of concentrated sulfuric acid was added to the blue product, the yellow color was formed. The absorbance of the yellow product was measured at λₘₐₓ=450 nm using the Clariostar microplate reader (BMG LABTECH). FIG. 22 shows the ELISA data.

### Fluorescence Microscope-Based Binding Assay

The 20 μL of 10 times diluted Hispur Ni-NTA resin bead (Ref #88221; Thermo scientific) was washed two times with 500 μL of SELEX buffer, resuspended in 50 μL of SELEX buffer, and incubated with 5 pmol of His-tagged target protein for 30 minutes at RT with rotation. The resin-protein complex was washed two times with 500 μL of SELEX buffer to remove unbound protein and resuspended with 50 μL of SELEX buffer. The 10 pmol 6-FAM-labeled ssDNA or aptamer was incubated in this resin-protein complex at RT for 30 min. After washing two times with 500 μL of SELEX buffer, the complex was finally resuspended in 50 μL of SELEX buffer and transferred on a glass slide for the fluorescence measurement. Fluorescence images were collected using both the green fluorescence and transmitted light channels by the digital inverted fluorescence microscope (Invitrogen EVOS FL).

### Determination of Binding Capability

The 100 nM His-tagged protein and anti-His-tagged biotinylated antibody were incubated at RT for 2 h in 50 μL of SELEX buffer. The protein-antibody complex was then incubated with 2 μL of sera-mag magnetic streptavidin-coated particles (Ref. #30152103010150; Cytiva) at RT for 2 h and stabilized overnight at 4° C. After washing two times with 200 μL of SELEX buffer, the protein-bead complex was incubated with the 6-FAM-labeled aptamer of 100 nM concentration in 1.5 mL Eppendorf tubes for 2 h. The unbound aptamer was removed by washing three times with 200 μL of SELEX buffer; the bound aptamer was eluted using 30 μL of hot SELEX buffer at 95° C. The fluorescence intensity from the sample at 520 nm was collected using the Clariostar microplate reader (BMG LABTECH).

### Synthesis of Gold Nanoparticles

A three-necked round-bottom flask was cleaned with freshly prepared aqua regia (concentrated HNO₃ and HCl in 1:3 molar ratio), rinsed with nuclease-free water, and perfectly dried before use. The AuNPs colloid was synthesized from KAuCl₄ (Ref. #334545-1G; Sigma Aldrich) precursor using the classical citrate reduction method. Briefly, 100 mL of 1 mM KAuCl₄ solution was heated to boiling. Then, 2 mL of 194 mM sodium citrate solution (CAS #1545801, Sigma Aldrich) was added, and the mixture was boiled for an additional 15 min with good stirring. The color of the solution changed from yellow, clear/gray, and finally to dark wine-red. After 15 min of boiling the reaction, the flask was taken out and cooled slowly to room temperature.

### Flow Cytometry Experiment to Measure the Binding Affinity of the Aptamers

The 100 μL of 200 nM His-tagged target protein was prepared in SELEX buffer and incubated with 1 μL of Ni-NTA magnetic beads (Ref #062N-A; G-Biosciences), rotating for 1 h at RT. The bead/protein complex was washed twice with 200 μL of SELEX buffer and incubated with 100 μL of 3, 10, 30, 100, 300, and 1000 nM of 6-FAM-labeled aptamer prepared in SELEX buffer for 1 h at RT with rotation. After incubation, the beads were washed two times with 200 μL of SELEX buffer and finally resuspended with 100 μL of SELEX buffer. The 6-FAM-labeled aptamers bound to the protein/bead complex were analyzed by Flow cytometry (Catalog #0500-4005; Guava easyCyte 5HT), counting approximately 5000 events. Each experiment was run for three trials to calculate the mean fluorescence intensity (X_{C}) and standard error. The binding affinity (K_{d}) of the 6-FAM labeled A1C1 aptamer against S1S2 was determined by an intensity vs. concentration plot using the Origin software.

Certain embodiments of the compositions, methods, and kits disclosed herein are defined in the above examples. It should be understood that these examples, while indicating particular embodiments of the invention, are given by way of illustration only. From the above discussion and these examples, one skilled in the art can ascertain the essential characteristics of this disclosure, and, without departing from the spirit and scope thereof, can make various changes and modifications to adapt the compositions, methods, and kits described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

1. A method of inhibiting binding between a coronavirus and a hACE2 receptor, the method comprising contacting a hACE2 receptor with a DNA aptamer to block binding to the hACE2 receptor, wherein the DNA aptamer binds to a spike protein of the coronavirus.

2. The method of claim 1, wherein the spike protein has a S1 subunit, a S2 subunit, and a S1S2 junction, and the DNA aptamer is specific for the S1 subunit, the S2 subunit, or the S1S2 junction.

3. The method of claim 1, wherein the DNA aptamer comprises S2A2C1, having a nucleotide sequence of AGGCGGGTTCCTAGACTTGTACTCAGCCT (SEQ ID NO: 1).

4. The method of claim 1, wherein the DNA aptamer comprises A1C1, having a nucleotide sequence of CGGGACGACGACGGACATCGTGAGAAATGGTCGACCTTGTGTCTGTCGTCCCG (SEQ ID NO: 2).

5. The method of claim 1, wherein the DNA aptamer comprises a fusion aptamer, wherein the fusion aptamer comprises a S1-specific aptamer fused to a S2-specific aptamer by a linker.

6. (canceled)

7. The method of claim 5, wherein the fusion aptamer comprises:
(i) S1B6C3, having a nucleotide sequence of CGCAGCACCCAAGAACAAGGACTGCTTAGGATTGCGATAGGTTCGG (SEQ ID NO: 3), linked to an additional aptamer;
(ii) S2A2C1, having a nucleotide sequence of AGGCGGGTTCCTAGACTTGTACTCAGCCT (SEQ ID NO: 1), linked to an additional aptamer; or
(iii) S1B6C3, having a nucleotide sequence of CGCAGCACCCAAGAACAAGGACTGCTTAGGATTGCGATAGGTTCGG (SEQ ID NO: 3), linked to S2A2C1, having a nucleotide sequence of AGGCGGGTTCCTAGACTTGTACTCAGCCT (SEQ ID NO: 1).

8-10. (canceled)

11. The method of claim 1, wherein the DNA aptamer comprises S1B6C3-A5-S2A2C1, wherein S1B6C3 has a nucleotide sequence of CGCAGCACCCAAGAACAAGGACTGCTTAGGATTGCGATAGGTTCGG (SEQ ID NO: 3), S2A2C1 has a nucleotide sequence of AGGCGGGTTCCTAGACTTGTACTCAGCCT (SEQ ID NO: 1), and A5 is a poly A linker.

12. The method of claim 1, wherein the hACE2 receptor is in a human subject.

13. (canceled)

14. The method of claim 1, wherein the coronavirus is SARS-CoV-2.

15-16. (canceled)

17. A method of treating a coronavirus infection, the method comprising administering to a subject having a coronavirus infection an effective amount of a DNA aptamer to inhibit binding between the coronavirus and hACE2 receptors in the subject so as to treat the coronavirus infection.

18. The method of claim 17, wherein the coronavirus infection is caused by SARS-CoV-2.

19-21. (canceled)

22. The method of claim 17, wherein the DNA aptamer comprises S2A2C1, having a nucleotide sequence of AGGCGGGTTCCTAGACTTGTACTCAGCCT (SEQ ID NO: 1).

23. The method of claim 17, wherein the DNA aptamer comprises A1C1, having a nucleotide sequence of CGGGACGACGACGGACATCGTGAGAAATGGTCGACCTTGTGTCTGTCGTCCCG (SEQ ID NO: 2).

24. The method of claim 17, wherein the DNA aptamer comprises a fusion aptamer, wherein the fusion aptamer comprises a S1-specific aptamer fused to a S2-specific aptamer by a linker.

25. (canceled)

26. The method of claim 23, wherein the fusion aptamer comprises:
(i) S1B6C3, having a nucleotide sequence of CGCAGCACCCAAGAACAAGGACTGCTTAGGATTGCGATAGGTTCGG (SEQ ID NO: 3), linked to an additional aptamer;
(ii) S2A2C1, having a nucleotide sequence of AGGCGGGTTCCTAGACTTGTACTCAGCCT (SEQ ID NO: 1), linked to an additional aptamer; or
(iii) S1B6C3, having a nucleotide sequence of CGCAGCACCCAAGAACAAGGACTGCTTAGGATTGCGATAGGTTCGG (SEQ ID NO: 3), linked to S2A2C1, having a nucleotide sequence of AGGCGGGTTCCTAGACTTGTACTCAGCCT (SEQ ID NO: 1).

26-28. (canceled)

29. The method of claim 17, wherein the DNA aptamer comprises S1B6C3-A5-S2A2C1, wherein S1B6C3 has a nucleotide sequence of CGCAGCACCCAAGAACAAGGACTGCTTAGGATTGCGATAGGTTCGG (SEQ ID NO: 3), S2A2C1 has a nucleotide sequence of AGGCGGGTTCCTAGACTTGTACTCAGCCT (SEQ ID NO: 1), and A5 is a poly A linker.

30. The method of claim 17, wherein the subject is a human subject.

31. A method of diagnosing a coronavirus infection, the method comprising:
obtaining a sample from a subject;
contacting the sample with a DNA aptamer specific for a spike protein of a coronavirus; and
analyzing an extent of binding between the DNA aptamer and the sample to determine if the coronavirus is present in the sample, wherein binding between the DNA aptamer and the sample indicates a coronavirus is present in the sample, so as to diagnose whether the subject has a coronavirus infection.

32. The method of claim 31, wherein the sample comprises mucus from a nose of the subject.

33. The method of claim 31, wherein the DNA aptamer comprises S2A2C1, having a nucleotide sequence of AGGCGGGTTCCTAGACTTGTACTCAGCCT (SEQ ID NO: 1), or A1C1, having a nucleotide sequence of CGGGACGACGACGGACATCGTGAGAAATGGTCGACCTTGTGTCTGTCGTCCCG (SEQ ID NO: 2).

34. (canceled)

35. The method of claim 31, wherein the DNA aptamer comprises a fusion aptamer, wherein the fusion aptamer comprises a S1-specific aptamer fused to a S2-specific aptamer by a linker.

36. (canceled)

37. The method of claim 35, wherein the fusion aptamer comprises:
(i) S1B6C3, having a nucleotide sequence of CGCAGCACCCAAGAACAAGGACTGCTTAGGATTGCGATAGGTTCGG (SEQ ID NO: 3), linked to an additional aptamer;
(ii) S2A2C1, having a nucleotide sequence of AGGCGGGTTCCTAGACTTGTACTCAGCCT (SEQ ID NO: 1), linked to an additional aptamer; or
(iii) S1B6C3, having a nucleotide sequence of CGCAGCACCCAAGAACAAGGACTGCTTAGGATTGCGATAGGTTCGG (SEQ ID NO: 3), linked to S2A2C1, having a nucleotide sequence of AGGCGGGTTCCTAGACTTGTACTCAGCCT (SEQ ID NO: 1).

38-40. (canceled)

41. The method of claim 31, wherein the DNA aptamer comprises:
S1B6C3-A5-S2A2C1, wherein S1B6C3 has a nucleotide sequence of CGCAGCACCCAAGAACAAGGACTGCTTAGGATTGCGATAGGTTCGG (SEQ ID NO: 3), S2A2C1 has a nucleotide sequence of AGGCGGGTTCCTAGACTTGTACTCAGCCT (SEQ ID NO: 1), and A5 is a poly A linker, or
S1B6C3-A5-S2A2C1, wherein S1B6C3 has a nucleotide sequence of CGCAGCACCCAAGAACAAGGACTGCTTAGGATTGCGATAGGTTCGG (SEQ ID NO: 3), S2A2C1 has a nucleotide sequence of AGGCGGGTTCCTAGACTTGTACTCAGCCT (SEQ ID NO: 1), and A5 is a poly A linker.

42-55. (canceled)


SEQUENCE LISTING (31 sequences)

SEQ ID NO: 1 - DNA, 29 bp, synthetic construct
  source 1..29: mol_type = other DNA
aggcgggttcctagacttgtactcagcct

SEQ ID NO: 2 - DNA, 53 bp, synthetic construct
  source 1..53: mol_type = other DNA
cgggacgacgacggacatcgtgagaaatggtcgaccttgtgtctgtcgtcccg

SEQ ID NO: 3 - DNA, 46 bp, synthetic construct
  source 1..46: mol_type = other DNA
cgcagcacccaagaacaaggactgcttaggattgcgataggttcgg

SEQ ID NO: 4 - DNA, 73 bp, synthetic construct
  source 1..73: mol_type = other DNA
caaggagcgaccagaggggcggtttatcaacaactcgctctgtacaccactctttgttggcatccttcagccc

SEQ ID NO: 5 - DNA, 73 bp, synthetic construct
  source 1..73: mol_type = other DNA
caaggagcgaccagaggcgggttcctagacttgtactcagcctttacagctatgccctggcatccttcagccc

SEQ ID NO: 6 - DNA, 46 bp, synthetic construct; identical to SEQ ID NO: 3
  source 1..46: mol_type = other DNA

SEQ ID NO: 7 - DNA, 56 bp, synthetic construct
  source 1..56: mol_type = other DNA
caaggagcgaccagaggggcggtttatcaacaactcgctctgtacaccactctttg

SEQ ID NO: 8 - DNA, 43 bp, synthetic construct
  source 1..43: mol_type = other DNA
caaggagcgaccagaggcgggttcctagacttgtactcagcct

SEQ ID NO: 9 - DNA, 90 bp, synthetic construct
  source 1..90: mol_type = other DNA
aggcgggttcctagacttgtactcagccttttttttttttttttcgcagcacccaagaacaaggactgcttaggattgcgataggttcgg

SEQ ID NO: 10 - DNA, 90 bp, synthetic construct
  source 1..90: mol_type = other DNA
cgcagcacccaagaacaaggactgcttaggattgcgataggttcggtttttttttttttttaggcgggttcctagacttgtactcagcct

SEQ ID NO: 11 - DNA, 90 bp, synthetic construct
  source 1..90: mol_type = other DNA
cgcagcacccaagaacaaggactgcttaggattgcgataggttcggaaaaaaaaaaaaaaaaggcgggttcctagacttgtactcagcct

SEQ ID NO: 12 - DNA, 80 bp, synthetic construct
  source 1..80: mol_type = other DNA
cgcagcacccaagaacaaggactgcttaggattgcgataggttcggaaaaaaggcgggttcctagacttgtactcagcct

SEQ ID NO: 13 - DNA, 100 bp, synthetic construct
  source 1..100: mol_type = other DNA
aggcgggttcctagacttgtactcagccttttttttttttttttttttttttttcgcagcacccaagaacaaggactgcttaggattgcgataggttcgg

SEQ ID NO: 14 - DNA, 100 bp, synthetic construct
  source 1..100: mol_type = other DNA
cgcagcacccaagaacaaggactgcttaggattgcgataggttcggtttttttttttttttttttttttttaggcgggttcctagacttgtactcagcct

SEQ ID NO: 15 - DNA, 85 bp, synthetic construct
  source 1..85: mol_type = other DNA
cgcagcacccaagaacaaggactgcttaggattgcgataggttcggaaaaaaaaaaaggcgggttcctagacttgtactcagcct

SEQ ID NO: 16 - DNA, 78 bp, synthetic construct
  source 1..78: mol_type = other DNA
ggaggctctcgggacgacgacggacatcgtgagaaatggtcgaccttgtgtctgtcgtcccgcctttaggatttacag

SEQ ID NO: 17 - DNA, 79 bp, synthetic construct
  source 1..79: mol_type = other DNA
ggaggctctcgggacgactctcgtagggatgggttgagggatagggttcgctgtgtcgtcccgcctttaggatttacag

SEQ ID NO: 18 - DNA, 73 bp, synthetic construct
  source 1..73: mol_type = other DNA
caaggagcgaccagaggggcggtttattaacaactcgctctgtacaccactctttgttggcatccttcagccc

SEQ ID NO: 19 - DNA, 73 bp, synthetic construct
  source 1..73: mol_type = other DNA
caaggagcgaccagaggtaacttcgacagctattaccgcaactacacccgtcatccgtggcatccttcagccc

SEQ ID NO: 20 - DNA, 72 bp, synthetic construct
  source 1..72: mol_type = other DNA
caaggagcgaccagaggcgggttcctagactttactcagcctttacaggtatgccctggcatccttcagccc

SEQ ID NO: 21 - DNA, 73 bp, synthetic construct
  source 1..73: mol_type = other DNA
  misc_difference 41: note = a, c, t, g, unknown, or other
caaggagcgaccagaggcgggttcctagacttgtactcagncttcacagctatgccctggcatccttcagccc

SEQ ID NO: 22 - DNA, 73 bp, synthetic construct
  source 1..73: mol_type = other DNA
caaggagcgaccagaggccaccgtttaccacgctttctcatccacaccccccggccatggcatccttcagccc

SEQ ID NO: 23 - DNA, 73 bp, synthetic construct
  source 1..73: mol_type = other DNA
caaggagcgaccagaggcgggttcctagacttgtacacagcctttacagctacgccctggcatccttcagccc

SEQ ID NO: 24 - DNA, 79 bp, synthetic construct
  source 1..79: mol_type = other DNA
ggaggctctcgggacgacgtgcaggcacaactaatgctaatgattgtctgtcatgtcgtcccgcctttaggatttacag

SEQ ID NO: 25 - DNA, 79 bp, synthetic construct
  source 1..79: mol_type = other DNA
ggaggctctcgggacgactaggcactgcaagggattactattccgacagtctacgtcgtcccgcctttaggatttacag

SEQ ID NO: 26 - DNA, 79 bp, synthetic construct
  source 1..79: mol_type = other DNA
ggaggctctcgggacgaccgccggtacggacatcgagtactttgtgtatccgcagtcgtcccgcctttaggatttacag

SEQ ID NO: 27 - DNA, 79 bp, synthetic construct
  source 1..79: mol_type = other DNA
ggaggctctcgggacgacgttcttcttggagggaggggatgtggggtgggatgggtcgtcccgcctttaggatttacag

SEQ ID NO: 28 - DNA, 15 bp, synthetic construct
  source 1..15: mol_type = other DNA
ttttttttttttttt

SEQ ID NO: 29 - DNA, 25 bp, synthetic construct
  source 1..25: mol_type = other DNA
ttttttttttttttttttttttttt

SEQ ID NO: 30 - DNA, 15 bp, synthetic construct
  source 1..15: mol_type = other DNA
aaaaaaaaaaaaaaa

SEQ ID NO: 31 - DNA, 10 bp, synthetic construct
  source 1..10: mol_type = other DNA
aaaaaaaaaa